(19) United States
(12) Patent Application Publication
(10) Pub. No.: US 2025/0057865 A1
(43) Pub. Date: Feb. 20, 2025

(54) ANTIVIRAL SUPPLEMENT FORMULATIONS FOR HERPESVIRUS INFECTIONS

(71) Applicant: Vymedic, Inc., Englewood, CO (US)

(72) Inventor: Cynthia A. WINNING, Parker, CO (US)

(21) Appl. No.: 18/802,718

(22) Filed: Aug. 13, 2024

Related U.S. Application Data
(60) Provisional application No. 63/519,358, filed on Aug. 14, 2023.
(60) Provisional application No. 63/667,464, filed on Jul. 3, 2024.

(51) Int. Cl. A61K 31/7048; A61K 31/198; A61K 31/375; A61K 31/4415; A61P 31/22

(52) U.S. Cl. CPC A61K 31/7048; A61K 31/198; A61K 31/375; A61K 31/4415; A61P 31/22

(57) ABSTRACT

Anti-herpesvirus supplement compositions, dosage forms, and methods are provided for treating, preventing, decreasing symptoms, and/or decreasing recurrence or reactivation of a herpesvirus infection. An anti-herpesvirus supplement composition is provided comprising a lysine, an ascorbic compound, a flavonoid glycoside, and a threonine, optionally comprising a taurine, and further optionally comprising a pyridoxine.

### CROSS-REFERENCES TO RELATED APPLICATIONS

This application claims the benefit of priority to U.S. Provisional Application No. 63/667,464, filed Jul. 3, 2024 and U.S. Provisional Application No. 63/519,358, filed Aug. 14, 2023, the disclosures of each of which are incorporated by reference herein in their entireties.

### FIELD OF THE INVENTION

Methods and antiviral supplement compositions are provided for treating, preventing, and/or preventing reactivation or recurrence of herpesvirus infections.

### BACKGROUND

Herpesvirus infections are chronic. Antiviral drugs typically do not eliminate the virus, but may ease symptoms, shorten duration of viral infection, and make the virus latent (inactive) to decrease risk of transmission.

Commonly used prescription antiviral drugs approved for herpes simplex virus 1 and 2, genital herpes, and varicella-zoster virus include acyclovir, famciclovir, and valacyclovir. Treatment with prescription antiviral drugs may be used to help sores heal during a first outbreak, lower frequency of recurrent outbreaks, lessen severity and duration of symptoms in recurrent outbreaks, or help reduce chance of passing a herpesvirus to a partner. Ganciclovir may be used to treat cytomegalovirus retinitis. There are limited options for treatment of Epstein-Barr virus or human herpesviruses 6, 7 or 8 infections. Typically, symptomatic treatment of disease is employed, such as use of medication for fever and body aches, fluid replacement, and use of antiviral medication such as ganciclovir and foscarnet.

Antiviral therapies for herpesvirus infections include nucleoside analogs such as acyclovir, penciclovir, valacyclovir, famciclovir, valomaciclovir, ganciclovir, valganciclovir, cidofovir, and brincidofovir that act by targeting a viral DNA polymerase. Nucleoside analogs can act as competitive inhibitors for naturally occurring nucleosides and nucleotides that are used by the viral DNA polymerases to transcribe the viral DNA. These agents require actively replicating virus to be able to exert their effect and may have no effect on nonreplicating latent virus. Foscarnet also exerts it activity via a viral DNA polymerase, but via a different mechanism. Foscarnet is a pyrophosphate analogue that binds directly to DNA polymerase and interferes with pyrophosphate binding required for DNA polymerase activity.

Disadvantages of nucleoside analogs may include drug resistance. Acyclovir resistance may develop in patients receiving long term suppressive therapy, especially in immunocompromised patients. Mutations that occur in the thymidine kinase gene that confer acyclovir resistance in HSV strains can also confer resistance to ganciclovir. Poole et al., 2018, Antiviral therapies for herpesviruses: current agents and new directions, Clin Ther 40 (8) 1282-1298. Foscarnet is approved for treatment of acyclovir-resistant mucocutaneous HSV infections in immunocompromised patients, but foscarnet is available only as an intravenous (IV) medication.

Acyclovir and valacyclovir are commonly used antivirals with good general tolerance. Despite their generally good safety profile, they can cause systemic adverse effects. One such adverse effect is acute renal failure associated with crystals in the renal tubule. Another side effect is neurotoxicity. Brandariz-Nunez et al., 2021, Neurotoxicity associated with acyclovir and valacyclovir: a systematic review of cases, Clinical Pharmacy and Therapeutics, doi 10.1111/jcpt.13464. Valacyclovir is widely prescribed for subjects infected with HSV. Valacyclovir is administered as an oral prodrug that converts to acyclovir in vivo, ultimately inhibiting DNA synthesis. Acyclovir is a purine nucleoside analog with inhibitory activity against HSV1, HSV2, and varicella-zoster virus. Valacyclovir is eliminated 89% renally and dose adjustment is required in renal impairment. Neurotoxicity can occur in some cases leading to confusion, delusions, anxiety, agitation and sensory impairment. Tarpey et al., Neurotoxicity secondary to valacyclovir, J Pharmacy Technol. 2022, vol 38(4), 251-252. One study found the risk of valacyclovir associated neurotoxicity in patients with hemodialysis and peritoneal dialysis is significantly higher in patients with normal renal function. Wang et al., (2022) Valacyclovir-associated neurotoxicity among patients on hemodialysis and peritoneal dialysis: A nationwide population-based study. Front. Med. 9:997379. doi: 10.3389/fmed.2022.997379. Alternatives to nucleoside analogs such as acyclovir or valacyclovir for treating herpesvirus infections are desirable.

Vaccines have been developed to prevent herpesvirus simplex virus 2, varicella-zoster virus, and cytomegalovirus. Krishnan et al., Frontiers in Microbiology, 2021, vol. 12, Article 798927; Gabutti et al., 2019, Immunotargets Ther vol. 8, 15-28; Anderholm et al., 2016, Drugs; 76 (17): 1625-1645. Passive immunization with immunoglobulin or hyperimmune globulin is used to either prevent infection or as an adjunct to antiviral therapy. Antiviral supplement compositions have been developed.

U.S. Pat. No. 4,424,232, Parkinson, discloses a method of treating herpes simplex comprising topically administering a composition comprising L-lysine, gibberellic acid, and optionally L-ascorbic acid.

WO 92/15315, Wilkinson, discloses a method for treating a herpes infection which comprises administering a composition comprising lysine, vitamin C and hesperidin.

US 2006/0173078, McGregor, discloses a composition including sodium citrate, 18 amino acids including L-lysine and L-threonine, calcium succinate, and alpha-lipoic acid for treatment of symptoms of a herpesvirus infection.

U.S. Pat. No. 7,790,203, Lowder, discloses a composition comprising zinc sulfate, magnesium sulfate, thiamin, riboflavin, pyridoxine hydrochloride, ascorbic acid, niacin, pantothenic acid, and lysine for treatment of herpetic epidermal lesions.

U.S. Pat. No. 8,236,768, Brown, discloses topical antiviral compositions comprising acyclovir and 2-deoxy-D-glucose.

U.S. Pat. No. 9,034,834, Vymedic, LLC, discloses a composition for treating an influenza A infection comprising a lysine, an ascorbic compound, pyridoxine HCl (vitamin B6), hesperidin, rutin, taurine and L-threonine. The composition was found to inhibit neuraminidase enzyme in a dose-dependent manner when compared to positive control Oseltimivir carboxylate (standard neuraminidase inhibitor, TAMIFLU® active moiety) and to decrease influenza A mRNA in influenza A-infected Vero cell supernatants.

U.S. Pat. No. 9,539,228, Rittenhouse, discloses a composition comprising lysine, zinc, selenium, vitamin B2, vitamin B6, vitamin B12, vitamin C, vitamin D3, folic acid, and bioflavonoids for treating herpes infections.

U.S. Pat. No. 10,682,324, Abreo, discloses topical compositions comprising acetyl cysteine, ascorbic acid, and glycerol.

US 2022/0378747 A1, Kelley, discloses a topical composition for treating skin wounds comprising 1-5 wt % zinc gluconate, 0.1-1.5 wt % lysine, 1-2 wt % taurine, 10 wt % glycerin, distilled water, and HCl to adjust to pH 7.

Alternative, economical, and effective antiviral supplement formulations for safely treating, preventing, and/or preventing reactivation of herpesvirus infections are desirable.

### SUMMARY

Oral and topical antiviral supplement compositions for treating, preventing, and/or preventing reactivation or recurrence of herpesvirus infections are provided. The composition comprises a therapeutically effective amount of a lysine, an ascorbic compound, a flavonoid glycoside, and a threonine. In some cases, the composition further comprises a pyridoxine and/or a taurine.

A method of treating, preventing, or preventing recurrence or reactivation of a herpesvirus infection in a subject in need thereof is provided, the method comprising administering to the subject a therapeutically effective amount of a composition comprising a lysine, an ascorbic compound, a flavonoid glycoside, and a threonine, optionally wherein the composition comprises a pyridoxine and/or a taurine. In some cases, reactivation may be determined by presence of anti-herpesvirus immunoglobulin (Ig) M in the subject.

In some cases, the herpesvirus is selected from the group consisting of herpes simplex type 1 (HSV-1), herpes simplex type 2 (HSV-2), varicella zoster virus (VZV), Epstein-Barr virus (EBV), human cytomegalovirus (HCMV), human herpesvirus 6A (HHV-6A), human herpesvirus 6B (HHV-6B), human herpesvirus 7 (HHV-7), and human herpesvirus 8 (HHV-8).

In some cases, the herpesvirus is selected from the group consisting of herpes simplex type 1 (HSV-1), herpes simplex type 2 (HSV-2), varicella zoster virus (VZV), and human cytomegalovirus (HCMV).

In some cases, the lysine is selected from L-lysine, L-lysine monohydrochloride, L-lysine dihydrochloride, L-lysine succinate, L-lysine glutamate, and L-lysine orotate.

In some cases, the ascorbic compound is selected from the group consisting of ascorbic acid, dehydroascorbic acid, calcium ascorbate, magnesium ascorbate, potassium ascorbate, sodium ascorbate, manganese ascorbate, zinc ascorbate, iron ascorbate, copper ascorbate, boron ascorbate, molybdenum ascorbate, chromium ascorbate, ascorbyl palmitate, ascorbyl arachidonate, ascorbyl stearate, ascorbyl linoleate, ascorbyl linolenate, and ascorbyl oleate.

In some cases, the flavonoid glycoside is selected the group consisting of hesperidin, rutin, naringin, and quercitrin, or a pharmacologically acceptable salt thereof. optionally wherein the flavonoid glycoside comprises hesperidin and rutin.

In some cases, the pyridoxine is selected from the group consisting of pyridoxine, pyridoxine hydrochloride, pyridoxine phosphate, pyridoxal, pyridoxal hydrochloride, pyridoxal 5-phosphate, pyridoxic acid, pyridoxamine, pyridoxamine hydrochloride, and pyridoxamine dihydrochloride.

In some cases, the composition comprises L-lysine monohydrochloride, ascorbic acid, calcium ascorbate, niacinamide ascorbate, ascorbyl palmitate, hesperidin, rutin, pyridoxine hydrochloride, threonine, and taurine.

In some cases, the composition comprises L-lysine monohydrochloride, ascorbic acid, calcium ascorbate, niacinamide ascorbate, ascorbyl palmitate, hesperidin, rutin, pyridoxal, threonine, and taurine.

A method of reducing herpesvirus replication in a cell is provided, the method comprising exposing a virus-infected cell to an effective amount of a composition comprising a lysine, an ascorbic compound, a flavonoid glycoside, a threonine, a pyridoxine, and optionally a taurine. In some cases, the pyridoxine is pyridoxal. In some cases, the pyridoxine is pyridoxal hydrochloride. In some cases, the pyridoxine is pyridoxine hydrochloride. In some cases, the pyridoxine is pyridoxal 5-phosphate. In some cases, the pyridoxine is pyridoxamine. In some cases, the pyridoxine is pyridoxamine hydrochloride. In some cases, the pyridoxine is pyridoxic acid.

An anti-herpesvirus supplement oral dosage form is provided, the composition comprising a therapeutically effective amount of a lysine, an ascorbic compound, a flavonoid glycoside, a threonine, a taurine, and a pyridoxine.

In some cases, the anti-herpesvirus effective amount of the lysine, such as L-lysine monohydrochloride, is in a single dose range of from about 1,000 mg to about 5,000 mg, 1,000 mg to 4,000 mg, 1,250 to 3,500 mg, 2,000 mg to 3,500 mg, or 2,000 to 3,000 mg.

In some cases, the dosage form is in a form selected from the group consisting of powder, capsule, lozenge, troche, gummy, tablet, orally disintegrating tablet, liquid, and caplet.

In some cases, the ascorbic compound is selected from the group consisting of ascorbic acid, dehydroascorbic acid, calcium ascorbate, niacinamide ascorbate, and ascorbyl palmitate. In some cases, the ascorbic compound comprises ascorbic acid, calcium ascorbate, niacinamide ascorbate, and ascorbyl palmitate. In some cases, the ascorbic compound comprises dehydroascorbic acid.

In some cases, the anti-herpesvirus dosage form further comprises one or more additives selected from the group consisting of vehicle, binder, disintegrating agent, lubricant, thickener, surfactant, osmotic pressure regulator, electrolyte, sweetener, flavoring, perfume, pigment, and pH regulator.

In some cases, the anti-herpesvirus dosage form further comprises a disintegrating agent selected from the group consisting of microcrystalline cellulose, carboxymethylcellulose (CMC), CMC-Na, CMC-Ca, and croscarmellose sodium.

In some cases, the anti-herpesvirus dosage form further comprises a lubricant selected from the group consisting of leucine, isoleucine, valine, sugar-ester, hardening oil, stearic acid, magnesium stearate, talc, and macrogol.

In some cases, the anti-herpesvirus dosage form further comprises a sweetener selected from the group consisting of glucose, fructose, maltose, sucrose, xylose, lactose, xylitol, sorbitol, mannitol, maltitol, xylitol, coupling sugar, paratinose, glycerin, erythritol, dextrin, cyclodextrin, fructo-oligosaccharide, galacto-oligosaccharide, lacto-sucrose, thaumatin, stevia, stevia extract, rebaudioside A, glycyrrhizinic acid, saccharin, alitame, and aspartame.

In some cases, the anti-herpesvirus composition or dosage form comprises in a single dose comprising from about 2 g to about 3.5 g L-lysine monohydrochloride; from about 0.1 g to about 1.5 g ascorbic acid; from about 0.2 g to about 0.8 g hesperidin; from about 0.1 g to about 0.5 g rutin; from about 0.04 g to about 0.08 g pyridoxine hydrochloride; from about 0.01 g to about 0.08 g threonine; and from about 0.02 g to about 0.4 g taurine. In some cases, the anti-herpesvirus dosage form further comprises from about 0.5 g to about 0.75 g calcium ascorbate; from about 0.1 g to about 0.5 g niacinamide ascorbate; and from about 0.01 g to about 0.1 g ascorbyl palmitate.

A method of reducing frequency of recurrence or reactivation of a herpesvirus infection in a subject is provided, the method comprising treating the subject with an effective amount of a composition comprising a therapeutically effective amount of a lysine, an ascorbic compound, a flavonoid glycoside, a threonine, a taurine, and a pyridoxine.

A method of decreasing severity and/or duration of symptoms of a herpesvirus infection in a subject is provided, the method comprising treating the subject with an effective amount of a composition comprising a therapeutically effective amount of a lysine, an ascorbic compound, a flavonoid glycoside, a threonine, a taurine, and a pyridoxine. The symptoms may be selected from the group consisting of skin rash, blisters, cold sores around lips, mouth, or tongue, genital sores, tingling, itching, burning, fever, headache, swollen lymph nodes, fatigue, muscle aches, and painful urination.

A method of reducing risk of or slowing development of Alzheimer's disease or dementia associated with a herpesvirus infection or reactivation of a herpesvirus infection in a subject is provided, the method comprising treating the subject with an effective amount of a composition comprising a therapeutically effective amount of a lysine, an ascorbic compound, a flavonoid glycoside, a threonine, a taurine, and a pyridoxine.

A composition is provided for treating, preventing, preventing reactivation, reducing symptom duration, reducing symptom severity of a herpesvirus infection, the composition comprising a lysine, an ascorbic compound, a flavonoid glycoside, a threonine, and optionally a pyridoxine, and/or optionally a taurine.

A composition for the manufacture of a medicament for treating, preventing, or preventing recurrence or preventing reactivation of a herpesvirus infection is provided, the composition comprising a lysine, an ascorbic compound, a flavonoid glycoside, a threonine, and optionally a pyridoxine, and/or a taurine.

### BRIEF DESCRIPTION OF THE DRAWINGS

FIG. 1A shows a graph of efficacy (i) of Test Composition 1 in ARPE-19 cells in unbuffered medium (DMEM) against infection with HSV-1. Test composition was added at the time of infection. Cell effect without virus (ii) of test composition in ARPE-19 cells is also shown.

FIG. 1B shows a graph of efficacy (i) of Test Composition 1 in ARPE-19 cells in bicarbonate-buffered medium (DMEM) against infection with HSV-1. Test composition was added at the time of infection. Cell effect without virus (ii) of test composition in ARPE-19 cells is also shown. The test composition exhibited a selectivity index (SI, CC50/EC50=SI) of 27 in ARPE-19 cells against infection with HSV-1 in bicarbonate-buffered medium (DMEM).

FIG. 1C shows a graph of efficacy of positive control acyclovir in ARPE-19 cells in unbuffered medium (DMEM) against infection with HSV-1. Positive control composition was added at the time of infection.

FIG. 1D shows a graph of efficacy of positive control acyclovir in ARPE-19 cells in bicarbonate-buffered medium (DMEM) against infection with HSV-1. Positive control composition was added at the time of infection.

FIG. 2A shows a graph of efficacy (i) of Test Composition 1 in ARPE-19 cells in unbuffered medium (DMEM) against infection with HSV-2. Test composition was added at the time of infection. Cell effect without virus (ii) of test composition in ARPE-19 cells is also shown.

FIG. 2B shows a graph of efficacy (i) of Test Composition in ARPE-19 cells in bicarbonate-buffered medium (DMEM) against infection with HSV-2. Test composition was added at the time of infection. Cell effect without virus (ii) of test composition in ARPE-19 cells is also shown. The test composition exhibited a selectivity index (SI, CC50/EC50=SI) of 17.7 in ARPE-19 cells against infection with HSV-2 in bicarbonate-buffered medium (DMEM).

FIG. 2C shows a graph of efficacy of positive control acyclovir in ARPE-19 cells in unbuffered medium (DMEM) against infection with HSV-2. Positive control composition was added at the time of infection.

FIG. 2D shows a graph of efficacy of positive control acyclovir in ARPE-19 cells in bicarbonate-buffered medium (DMEM) against infection with HSV-2. Positive control composition was added at the time of infection.

FIG. 3A shows a graph of efficacy (i) of Test Composition 1 in ARPE-19 cells in unbuffered medium (DMEM) against infection with varicella zoster virus (VZV). Test composition was added at the time of infection. Cell effect without virus (ii) of test composition in ARPE-19 cells is also shown.

FIG. 3B shows a graph of efficacy (i) of Test Composition 1 in ARPE-19 cells in bicarbonate-buffered medium (DMEM) against infection with VZV. Test composition was added at the time of infection. Cell effect without virus (ii) of test composition in ARPE-19 cells is also shown.

FIG. 3C shows a graph of efficacy (i) of positive control cidofovir in ARPE-19 cells in unbuffered medium (DMEM) against infection with VZV. Positive control composition was added at the time of infection. Cell effect without virus of positive control (ii) in ARPE-19 cells is also shown.

FIG. 3D shows a graph of efficacy (i) of positive control cidofovir in ARPE-19 cells in bicarbonate-buffered medium (DMEM) against infection with VZV. Positive control composition was added at the time of infection. Cell effect without virus of positive control (ii) in ARPE-19 cells is also shown.

FIG. 4A shows a graph of efficacy (i) of Test Composition 1 in human foreskin fibroblasts (HFFs) cells in unbuffered medium (DMEM) against infection with human cytomegalovirus (HCMV). Test composition was added at the time of infection. Cell effect without virus (ii) of test composition in HFF cells is also shown.

FIG. 4B shows a graph of efficacy (i) of Test Composition 1 in HFF cells in bicarbonate-buffered medium (DMEM) against infection with HCMV. Test composition was added at the time of infection. Cell effect without virus (ii) of test composition in HFF cells is also shown.

FIG. 5A-L show test results for HSV1 in ARPE-19 cells with buffered medium and the associated curves at various concentrations of 11 different test compositions and positive control acyclovir: solid circles are efficacy data points in presence of virus for test compositions plotted as L-lysine HCl concentration (uM) (test compositions 1-11) or acyclovir concentration (uM) vs HSV1 yield (%); solid squares are cell effect data points in absence of virus for L-lysine HCl concentration (uM) vs cell density (%).

FIG. 5A shows test results for HSV1 in ARPE-19 cells with buffered medium and the associated curves at various concentrations of test composition 1: solid circles are efficacy data points in presence of virus for test composition plotted as L-lysine HCl concentration (uM) vs HSV1 yield (%); solid squares are cell effect data points in absence of virus for L-lysine HCl concentration (uM) vs cell density (%).

FIG. 5B shows test results for HSV1 in ARPE-19 cells with buffered medium and the associated curves at various concentrations of test composition 2: solid circles are efficacy data points in presence of virus for test composition plotted as L-lysine HCl concentration (uM) vs HSV1 yield (%); solid squares are cell effect data points in absence of virus for L-lysine HCl concentration (uM) vs cell density (%).

FIG. 5C shows test results for HSV1 in ARPE-19 cells with buffered medium and the associated curves at various concentrations of test composition 3: solid circles are efficacy data points in presence of virus for test composition plotted as L-lysine HCl concentration (uM) vs HSV1 yield (%); solid squares are cell effect data points in absence of virus for L-lysine HCl concentration (uM) vs cell density (%).

FIG. 5D shows test results for HSV1 in ARPE-19 cells with buffered medium and the associated curves at various concentrations of test composition 4: solid circles are efficacy data points in presence of virus for test composition plotted as L-lysine HCl concentration (uM) vs HSV1 yield (%); solid squares are cell effect data points in absence of virus for L-lysine HCl concentration (uM) vs cell density (%).

FIG. 5E shows test results for HSV1 in ARPE-19 cells with buffered medium and the associated curves at various concentrations of test composition 5: solid circles are efficacy data points in presence of virus for test composition plotted as L-lysine HCl concentration (uM) vs HSV1 yield (%); solid squares are cell effect data points in absence of virus for L-lysine HCl concentration (uM) vs cell density (%).

FIG. 5F shows test results for HSV1 in ARPE-19 cells with buffered medium and the associated curves at various concentrations of test composition 6: solid circles are efficacy data points in presence of virus for test composition plotted as L-lysine HCl concentration (uM) vs HSV1 yield (%); solid squares are cell effect data points in absence of virus for L-lysine HCl concentration (uM) vs cell density (%).

FIG. 5G shows test results for HSV1 in ARPE-19 cells with buffered medium and the associated curves at various concentrations of test composition 7: solid circles are efficacy data points in presence of virus for test composition plotted as L-lysine HCl concentration (uM) vs HSV1 yield (%); solid squares are cell effect data points in absence of virus for L-lysine HCl concentration (uM) vs cell density (%).

FIG. 5H shows test results for HSV1 in ARPE-19 cells with buffered medium and the associated curves at various concentrations of test composition 8: solid circles are efficacy data points in presence of virus for test composition plotted as L-lysine HCl concentration (uM) vs HSV1 yield (%); solid squares are cell effect data points in absence of virus for L-lysine HCl concentration (uM) vs cell density (%).

FIG. 5I shows test results for HSV1 in ARPE-19 cells with buffered medium and the associated curves at various concentrations of test composition 9: solid circles are efficacy data points in presence of virus for test composition plotted as L-lysine HCl concentration (uM) vs HSV1 yield (%); solid squares are cell effect data points in absence of virus for L-lysine HCl concentration (uM) vs cell density (%).

FIG. 5J shows test results for HSV1 in ARPE-19 cells with buffered medium and the associated curves at various concentrations of test composition 10: solid circles are efficacy data points in presence of virus for test composition plotted as L-lysine HCl concentration (uM) vs HSV1 yield (%); solid squares are cell effect data points in absence of virus for L-lysine HCl concentration (uM) vs cell density (%).

FIG. 5K shows test results for HSV1 in ARPE-19 cells with buffered medium and the associated curves at various concentrations of test composition 11: solid circles are efficacy data points in presence of virus for test composition plotted as L-lysine HCl concentration (uM) vs HSV1 yield (%); solid squares are cell effect data points in absence of virus for L-lysine HCl concentration (uM) vs cell density (%).

FIG. 5L shows comparative results for HSV1 in ARPE-19 cells with buffered medium and the associated curves at various concentrations of positive control acyclovir: solid circles are efficacy data points in presence of virus for test composition plotted as acyclovir (uM) vs HSV1 yield (%); solid squares are cell effect data points in absence of virus for acyclovir (uM) vs cell density (%).

FIG. 6A-L show test results for HSV2 in ARPE-19 cells with buffered medium and the associated curves at various concentrations of 11 different test compositions and positive control acyclovir: solid circles are efficacy data points in presence of virus for test compositions plotted as L-lysine HCl concentration (uM) (test compositions 1-11) or acyclovir concentration (uM) vs HSV2 yield (%); solid squares are cell effect data points in absence of virus for L-lysine HCl concentration (uM) vs cell density (%).

FIG. 6A shows test results for HSV2 in ARPE-19 cells with buffered medium and the associated curves at various concentrations of test composition 1: solid circles are efficacy data points in presence of virus for test composition plotted as L-lysine HCl concentration (uM) vs HSV2 yield (%); solid squares are cell effect data points in absence of virus for L-lysine HCl concentration (uM) vs cell density (%).

FIG. 6B shows test results for HSV2 in ARPE-19 cells with buffered medium and the associated curves at various concentrations of test composition 2: solid circles are efficacy data points in presence of virus for test composition plotted as L-lysine HCl concentration (uM) vs HSV2 yield (%); solid squares are cell effect data points in absence of virus for L-lysine HCl concentration (uM) vs cell density (%).

FIG. 6C shows test results for HSV2 in ARPE-19 cells with buffered medium and the associated curves at various concentrations of test composition 3: solid circles are efficacy data points in presence of virus for test composition plotted as L-lysine HCl concentration (uM) vs HSV2 yield (%); solid squares are cell effect data points in absence of virus for L-lysine HCl concentration (uM) vs cell density (%).

FIG. 6D shows test results for HSV2 in ARPE-19 cells with buffered medium and the associated curves at various concentrations of test composition 4: solid circles are efficacy data points in presence of virus for test composition plotted as L-lysine HCl concentration (uM) vs HSV2 yield (%); solid squares are cell effect data points in absence of virus for L-lysine HCl concentration (uM) vs cell density (%).

FIG. 6E shows test results for HSV2 in ARPE-19 cells with buffered medium and the associated curves at various concentrations of test composition 5: solid circles are efficacy data points in presence of virus for test composition plotted as L-lysine HCl concentration (uM) vs HSV2 yield (%); solid squares are cell effect data points in absence of virus for L-lysine HCl concentration (uM) vs cell density (%).

FIG. 6F shows test results for HSV2 in ARPE-19 cells with buffered medium and the associated curves at various concentrations of test composition 6: solid circles are efficacy data points in presence of virus for test composition plotted as L-lysine HCl concentration (uM) vs HSV2 yield (%); solid squares are cell effect data points in absence of virus for L-lysine HCl concentration (uM) vs cell density (%).

FIG. 6G shows test results for HSV2 in ARPE-19 cells with buffered medium and the associated curves at various concentrations of test composition 7: solid circles are efficacy data points in presence of virus for test composition plotted as L-lysine HCl concentration (uM) vs HSV2 yield (%); solid squares are cell effect data points in absence of virus for L-lysine HCl concentration (uM) vs cell density (%).

FIG. 6H shows test results for HSV2 in ARPE-19 cells with buffered medium and the associated curves at various concentrations of test composition 8: solid circles are efficacy data points in presence of virus for test composition plotted as L-lysine HCl concentration (uM) vs HSV2 yield (%); solid squares are cell effect data points in absence of virus for L-lysine HCl concentration (uM) vs cell density (%).

FIG. 6I shows test results for HSV2 in ARPE-19 cells with buffered medium and the associated curves at various concentrations of test composition 9: solid circles are efficacy data points in presence of virus for test composition plotted as L-lysine HCl concentration (uM) vs HSV2 yield (%); solid squares are cell effect data points in absence of virus for L-lysine HCl concentration (uM) vs cell density (%).

FIG. 6J shows test results for HSV2 in ARPE-19 cells with buffered medium and the associated curves at various concentrations of test composition 10: solid circles are efficacy data points in presence of virus for test composition plotted as L-lysine HCl concentration (uM) vs HSV2 yield (%); solid squares are cell effect data points in absence of virus for L-lysine HCl concentration (uM) vs cell density (%).

FIG. 6K shows test results for HSV2 in ARPE-19 cells with buffered medium and the associated curves at various concentrations of test composition 11: solid circles are efficacy data points in presence of virus for test composition plotted as L-lysine HCl concentration (uM) vs HSV2 yield (%); solid squares are cell effect data points in absence of virus for L-lysine HCl concentration (uM) vs cell density (%).

FIG. 6L shows comparative results for HSV2 in ARPE-19 cells with buffered medium and the associated curves at various concentrations of positive control acyclovir: solid circles are efficacy data points in presence of virus for test composition plotted as acyclovir (uM) vs HSV2 yield (%); solid squares are cell effect data points in absence of virus for acyclovir (uM) vs cell density (%).

FIG. 7 shows a Table of various test solutions with components. The two most effective test solutions vs HSV1 and HSV2 were the original Test Composition (Solution 1) and Solution 7.

FIG. 8 shows an overlay of efficacy curves against HSV1 in ARPE-19 cells with buffered medium for test solutions 1-11. Solution 1 and Solution 7 were the most efficacious test solutions against HSV1.

FIG. 9 shows an overlay of efficacy curves against HSV1 in ARPE-19 cells with buffered medium for test solutions 1, 2, 3, 4 and 7.

FIG. 10 shows an overlay of efficacy curves against HSV2 in ARPE-19 cells with buffered medium for test solutions 1-11. Solution 1, Solution 4, and Solution 7 were the most efficacious test solutions against HSV2.

FIG. 11 shows an overlay of efficacy curves against HSV2 in ARPE-19 cells with buffered medium for test solutions 1, 2, 3, 4 and 7.

FIG. 12 shows a timeline for a safety study in human subjects.

### DETAILED DESCRIPTION OF THE INVENTION

Antiviral supplement compositions are provided for treating, preventing, reducing recurrence, reducing severity, and/or reducing duration of a herpesvirus infection. Antiviral supplement compositions are provided comprising a lysine, an ascorbic compound, a flavonoid glycoside, a threonine, and a pyridoxine. In some cases, the composition includes L-lysine hydrochloride, ascorbic acid, calcium ascorbate, niacinamide ascorbate, ascorbyl palmitate, rutin, hesperidin, pyridoxine hydrochloride, taurine, and threonine.

The term “patient” or “subject” as used herein refers to an animal, for example a mammal, such as a human, who is the object of treatment. The subject, or patient, may be either male or female.

The term “virus” used herein refers to any of a large group of submicroscopic agents that consist of a segment of DNA or RNA surrounded by a coat of protein. Herpesviridae viruses are DNA viruses. The virus requires a host cell to replicate. Because viruses are unable to replicate without a host cell, they are not considered living organisms in conventional taxonomic systems. They are described as “live” when they are capable of replicating and causing disease. Accordingly, the term “viral activity” refers to any state of being active or any energetic action or movement or liveliness of a virus. Accordingly, the term “viral replication” refers to any process by which genetic materials, a single-celled organism, or a virus reproduces or makes a copy of itself.

The singular forms “a”, “an” and “the” are intended to include the plural forms as well, unless the context clearly indicates otherwise.

The term “and/or” refers to and encompasses any and all possible combinations of one or more of the associated listed items.

The term “about,” when referring to a measurable value such as an amount of a compound, dose, time, temperature, and the like, is meant to encompass variations of +/−10%, 5%, 1%, 0.5%, or even 0.1% of the specified amount.

The terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Unless otherwise defined, all terms, including technical and scientific terms used in the description, have the same meaning as commonly understood by one of ordinary skill in the art to which this disclosure belongs. In the event of conflicting terminology, the present specification is controlling.

The term, “pharmaceutically acceptable carrier” as used herein means a pharmaceutically acceptable material, composition or vehicle, such as a liquid or solid filler, diluent, excipient, solvent or encapsulating material, involved in carrying or transporting the compositions of the invention from one organ, or portion of the body, to another organ, or portion of the body without affecting its biological effect. Each carrier should be “acceptable” in the sense of being compatible with the other ingredients of the composition and not injurious to the subject.

As used herein, the term “pharmaceutically acceptable salt” refers to those salts which retain the biological effectiveness and properties of the active ingredient of the biochemical composition, which are not otherwise undesirable. Pharmaceutically acceptable salts include, but are not limited to, salts of sodium, potassium, calcium, magnesium, aluminum, and the like.

The term “infection” as used herein refers to the presence of a virus in or on a subject, which if replication of the virus was retarded or of the activity of the virus was reduced, would result in a benefit to the subject. Accordingly, the term “infection” refers to the presence of pathogens at any anatomical site of a human or animal.

The term “treating” as used herein refers to the administration of a compound or composition to a subject for therapeutic purposes. The term “administration” includes delivery to a subject by any appropriate method which serves to deliver the drug to the site of the infection. The administration of the drug can be oral, nasal, parental, topical, ophthalmic, transmucosal, or transdermal administration or delivery in the form of solid, semi-solid, lyophilized powder, or liquid dosage forms. The dosage forms include tablets, capsules, troches, powders, solutions, suspensions, suppositories, ointment, lip balm, cream, paste, gel, lotion, emulsions, and the like, preferably in unit dosage forms suitable for simple administration of precise dosages.

The term “antiviral supplement” as used herein includes any composition used specifically for treatment or prophylaxis of viral infections, particularly herpesvirus infections. The compositions of the disclosure may be evaluated in one aspect by cell-based antiviral assays. The compositions of the disclosure may be evaluated by retarding the growth and reproduction of viruses in a cell-based assay. The compositions of the disclosure may be evaluated by decreasing the duration of a viral infection in a patient. In another aspect, the compositions of the disclosure can be evaluated by decreasing the severity or duration of symptoms of a viral infection in a patient.

As used herein, the term “an effective amount” refers to that an amount of a composition of the disclosure that when administered to an individual subject in need thereof, is sufficient to reduce the virus activity and/or growth thereby enhancing the antiviral activity.

As used herein, the term “therapeutically effective amount” refers to an amount of a composition of the disclosure that when administered to a human subject in need thereof, is sufficient to effect treatment or prophylaxis for herpesvirus infection. The amount that is therapeutically effective will depend upon the patient's size and gender, the stage and severity of the infection and the result sought. For a given patient and condition, a therapeutically effective amount can be determined by methods known to those of skill in the art. For example, in reference to the treatment of a herpesvirus infection using the compositions of the present invention, a therapeutically effective amount refers to that amount of the composition which has the effect of (1) reducing the shedding of the virus, (2) reducing the duration of the infection, (3) reducing infectivity and/or, (4) reducing the severity (or, preferably, eliminating) one or more other symptoms associated with the infection such as, for example, skin rash, blisters, cold sores around lips, mouth, or tongue, genital sores, tingling, itching, or burning, fever, headache, swollen lymph nodes, fatigue, muscle aches, and painful urination. Such an effective dose will generally depend on the factors described above. A prophylactically effective dose is one that reduces the likelihood of contacting a herpesvirus infection. A prophylactically effective dose is from about 20% to about 100%, preferably from about 40% to about 60%, of a therapeutically effective dose.

The term “CC50” refers to the concentration that reduces the number of viable cells by 50% compared with the control.

The term “EC50” refers to half maximal effective concentration, or the concentration that causes half of the maximum possible effect.

In compositions of the disclosure, the CC50 and EC50 values can be calculated based on Lysine equivalent concentrations.

All patents, patent applications and publications referred to herein are incorporated by reference in their entirety.

Compositions and methods are provided for treating, preventing, and/or preventing reactivation of herpesvirus infections. Compositions and methods are provided to shorten healing during first outbreak, lower frequency of recurrent outbreaks, lessen severity and duration of symptoms in recurrent outbreaks, and reduce chance of passing a herpesvirus to a partner.

### Herpesviruses

Of the more than 100 know herpesviruses at least nine herpesvirus types are known to infect humans: herpes simplex viruses 1 and 2 (HSV-1 and HSV-2, also known as HHV-1 and HHV-2), varicella zoster virus (human herpesvirus 3, HHV-3), Epstein-Barr virus (EBV, or HHV-4), human cytomegalovirus (HCMV, or HHV-5), human herpesvirus 6A and 6B (HHV-6A and HHV-6B), human herpesvirus 7 (HHV-7), and Kaposi's sarcoma-associated herpesvirus (KSHV, or human herpesvirus 8, HHV-8).

Herpes viruses are divided into three groups: the alpha herpesviruses, herpes simplex virus types 1 and 2, and varicella-zoster virus, have a short replicative cycle, induce cytopathology in monolayer cell cultures, and have a broad host range; beta herpesviruses, cytomegalovirus, and human herpesviruses 6 and 7, with a long replicative cycle and restricted host range; and gamma herpesviruses, Epstein-Barr virus and human herpesvirus 8, with a very restricted host range. A simian virus, called B virus, occasionally infects humans. All herpes viruses can establish a latent infection within specific tissues, which are characteristic for each virus.

Transcription, genome replication, and capsid assembly occur in the host cell nucleus. Genes are replicated in a specific order: (1) immediate-early genes, which encode regulatory proteins; (2) early genes, which encode enzymes for replicating viral DNA; and (3) late genes, which encode structural proteins. The tegument and envelope are acquired as the virion buds out through the nuclear membrane or endoplasmic reticulum. Virions are transported to the cell membrane via the Golgi apparatus, and the host cell dies as mature virions are released. Alternatively, in selected cell types, the virus may be maintained in a latent state. The latent viral genome may reactivate at any time. The mechanism of reactivation is not known.

Cytomegalovirus retinitis is diagnosed clinically. Diagnosis of other types of herpesvirus infection relies on isolation of the virus through culturing, and/or detection of viral genes or gene products, for example, using polymerase chain reaction technology.

Herpes simplex viruses 1 and 2 (HSV-1 and HSV-2) both cause orolabial and genital herpes. Herpes simplex viruses have about 50 percent genomic homology. Both types of herpes simplex virus can cause oral or genital infection. HSV-1 typically cause gingivostomatitis, herpes labialis, and herpes keratitis. HSV-2 typically causes genital lesions.

Mucocutaneous manifestations of herpes simplex virus infection may include gingivostomatitis, herpes genitalis, herpetic keratitis, and dermal whitlows. Neonatal herpes simplex virus infection and herpes simplex virus encephalitis also occur. The virus replicates mainly in epithelial cells producing a characteristic vesicle on an erythematous base. It then ascends sensory nerves to the dorsal root ganglia, where after a period of replication it establishes latency. During reactivated infection, the virus spreads distally from the ganglion to initiate new cutaneous and/or mucosal lesions. Herpes simplex virus 1 transmission is primarily oral, and herpes simplex virus 2 virus is primarily genital. Transmission requires intimate contact. Symptoms of herpes simplex virus infection may include cold sores, oral blistering sores, fever blisters, genital blistering sores, itching, pain during urination, fever, headache, fatigue, lack of appetite. HSV infection can cause swelling and inflammation within the organs associated with sexual activity and urination. These include ureter, rectum, cervix, and uterus. In recurrent outbreaks, prodromal symptoms may occur a few hours or days before a new outbreak starts. Prodromal symptoms may include genital pain, tingling or shooting pain in the legs, buttocks or hips. An HSV infection can spread to a finger through a break in the skin, causing discoloration, swelling and sores; the infection is called herpetic whitlow. HSV infection of the eye can cause pain, sores, blurred vision, and blindness. Rarely, HSV infection may lead to inflammation and swelling of the brain, called encephalitis. Rarely, HSV in the bloodstream can cause infections of the internal organs.

Evidence indicates that herpes simplex virus (HSV) may participate in the pathogenesis of Alzheimer's disease (AD). A growing body of evidence suggests that herpes simplex virus type 1 (HSV1) might play a considerable role in the development of AD. Longitudinal cohort studies have shown that reactivation of HSV, measured as immunoglobulin (Ig) M-positivity for HSV, increases the risk of developing AD approximately 8-10 years after HSV reactivation. Reactivation of HSV seems to have a stronger correlation to AD than HSV1 carriage alone. Further, population-based cohort studies indicate that the genetic background, and allele 4 of the apolipoprotein E gene (APOE e4) in particular may interact with HSV1 infections to increase risk of AD. APOE e4 carriers have an increased risk of cold sores. HSV has the ability to establish lifelong latency in sensorial ganglia and can reactivate recurrently giving rise to symptoms from oral ulcers to encephalitis, providing clinical evidence for the ability of the virus to reach the brain. Signatures from herpesviruses, including HSV1, were found when transcriptomes of brain samples from AD patients were analyzed. Recent findings suggest HSV1 may reactivate subclinically and migration to the brain without any symptoms of encephalitis is possible. Hemmingsson et al., 2021, Antiviral treatment associated with reduced risk of clinical Alzheimer's disease-a nested case-control study, Alzheimer's & Dement. 2021; 7:e12187.

Hemmingsson et al., 2021 investigated if antiviral treatment given prior to onset of AD could influence incident AD. From a large population-based cohort study in Northern Sweden, 262 individuals that later developed AD were compared to non-AD matched control group with respect to prescriptions of herpes antiviral treatment. All subjects were HSV1 carriers and the matching criteria were age, sex, APOE e4 carriership, and study sample start year. Among those who developed AD, 6 prescriptions of antivirals were found, compared to 20 among matched controls. Adjusted for length of follow-up, a conditional logistic regression indicated difference in the risk for AD development between groups (odds ratio for AD with antiviral prescription 0.287, P=0.018). Antiviral treatment might possibly reduce the risk for later development of HSV1-associated AD.

Much evidence indicates that HSV1 can enter the brain and can reside there in latent form. Stress, inflammation, and other events can lead to reactivation of the virus causing a productive infection and consequent damage which is suggested to be greater in people who carry the type 4 allele of the apolipoprotein E gene (APOE-e4). HSV1 in brain in combination with carriage of APOE-34 allele can confer a high risk of developing AD. ASV1 in brain of non-APOE-34 carriers confers a much lower or no risk. Itzhaki, Ruth F. Overwhelming evidence for a major role for Herpes Simplex Virus Type 1 (HSV1) in Alzheimer's Disease (AD); underwhelming evidence against, Vaccines, 2021, 9, 679.

Vestin et al. investigated AD and dementia risks according to presence of herpesvirus antibodies in relation to anti-herpesvirus treatment and potential APOE e4 carriership interaction. 1002 dementia-free 70-year olds living in Sweden in 2001-2005 were followed for 15 years. Serum samples were analyzed to detect anti-HSV and anti-HSV1 immunoglobulin (Ig) G, ani-cytomegalovirus (CMV) IgG, anti-HSV IgM, and anti-HSV and anti-CMV IgG levels. Diagnoses and prescriptions were collected from medical records. Cox proportional-hazard regression models were applied. Cumulative AD and all-cause dementia incidences were 4% and 7%, respectively. Eighty-two percent of participants were anti-HSV IgG carriers, of whom 6% received anti-herpesvirus treatment. Anti-HSV IgG was associated with a more than double dementia risk. No significant association was found with AD. No interaction between anti-HSV IgG seroprevalence and APOE e4 carriership or anti-CMV IgG seroprevalence was found. Vestin et al., Herpes simplex viral infection doubles the risk of dementia in a contemporary cohort of older adults: a prospective study. J Alzheimer's Disease 97 (2024) 1841-1850.

An “infection hypothesis” of the etiology of Alzheimer's disease has been proposed. Beta-amyloid peptide (Ab) has been found to be an antimicrobial peptide (AMP) acting against bacteria, fungi, and viruses. Microbial infection has been shown to increase the synthesis of this AMP. It has been proposed that the production of Ab as an AMP will be beneficial on first microbial challenge but will become progressively detrimental as the infection becomes chronic and reactivates from time to time. Fulop et al., 2018, Can an infection hypothesis explain the beta amyloid hypothesis of Alzheimer's disease? Frontiers in Aging Neuroscience, Vol. 10, article 224. Fulop proposes that host measures to remove excess Ab decrease over time due to microglial senescence and microbial film formation. People infected by HSV-1 show some decline of the immune system with age which enables HSV-1 to migrate from the periphery to the brain, or alternatively, in stressful circumstances, HSV-1 may infects the brain directly via the olfactory route. Once HSV-1 is in the brain it is able to facilitate processes that contribute to neuroinflammation. It has been proposed that HSV-1 in the brain contributes to abnormal processing of amyloid precursor protein (APP) to Ab and favors aggregation.

Without being bound by theory, a herpes breakthrough activation of the dormant virus in the trigeminal ganglion may travel to the lip using the alveolar nerve. At the same time, the HSV virus may travel to the brain using the trigeminal nerve. The immune response to a brain infection may involve Ab to destroy the virus. Over time, recurrent HSV reactivation may contribute to Alzheimer's disease. An anti-herpesvirus composition that can cross the blood brain barrier may decrease HSV reactivation and thereby slow progression of Alzheimer's disease.

Varicella zoster virus (VZV), also known as human herpesvirus 3 or human alphaherpesvirus 3, infection causes varicella (chicken pox). Reactivation of latent virus (usually in adults) causes herpes zoster (shingles), manifesting as vesicular rash with dermatomal distribution and acute neuritis. Varicella-zoster virus is highly contagious and may be transmitted by droplets and replicates in nasopharynx. Latency is established in dorsal root ganglia and virus reinfection results in virion transport down sensory nerves. Symptoms of VZV infection may include rash, fever, loss of appetite, headache, fatigue. A rash may appear 10 to 21 days after exposure to the varicella-zoster virus. The rash may last for 5 to 10 days. The rash may go through three phases including raised bumps called papules which break in a few days; small fluid-filled blisters called vesicles which form in about one day then break and leak; and crusts and scabs which cover broken blisters and take a few days to heal. Shingles, also known as herpes zoster, is caused by VZV recurrence, and typically causes symptoms including a painful blistering rash. It can occur in older adults or people with weakened immune systems. The rash may appear on one side of the body or face and last two to four weeks. Shingles may cause chronic, debilitating pain that can linger after the rash clears up. A less common complication affects the eye in up to one in four cases, and may result in prolonged pain, facial scarring, and in some cases vision loss.

Human cytomegalovirus (HCMV) is a leading cause of viral congenital birth defects. Human cytomegalovirus infection in children and adults can cause clinical symptoms mononucleosis syndrome with fever, malaise, atypical lymphocytosis, and pharyngitis. Cytomegalovirus replicates in the salivary glands and kidneys and is shed in urine and saliva. Replication is slow. Transmission is via intimate contact with infected secretions. Cytomegalovirus infections are among the most prevalent viral infections worldwide. Symptoms of cytomegalovirus infection may include fever, night sweats, tiredness and uneasiness, sore throat, joint and muscle pain, low appetite, weight loss, and/or mouth ulcers. In babies, CMV infection may cause symptoms of low birth weight, jaundice, pneumonia, poor functioning of liver, prolonged fever, inflammation of the brain, and/or seizures.

Epstein-Barr virus causes classic mononucleosis. In immunocompromised hosts, the virus causes lymphoproliferative syndrome. Epstein-Barr virus replicates in the epithelial cells of the oropharynx and in beta lymphocytes. Epstein Barr virus is transmitted by intimate contact, particularly exchange of saliva. Epstein-Barr virus infection can cause symptoms including fever, fatigue, exhaustion, sore throat, throat inflammation, swollen lymph nodes in neck and armpits, rash, headache, muscle aches, enlarged spleen and liver, lack of appetite, and/or abdominal pain.

Human betaherpesviruses 6A, 6B, and 7 are associated with exanthem subitem (roseola) and with rejection of transplanted kidneys. Human herpesvirus 6A infection symptoms may include fever, rash, diarrhea, liver problems such as hepatitis, low blood counts, enlarged lymph nodes, longstanding fatigue, and/or neurological signed if the brain is affected. Human herpesviruses 6B primarily infects infants. Symptoms may include fever and roseola infantum with a possible complication of febrile seizures. Recurrence of human herpesviruses 6B infection in adults may be associated with glandular fever syndrome, where there is swollen lymph nodes, chronic fatigue, inflamed liver (hepatitis). Complications such as bone marrow or encephalitis may occur. Human betaherpesvirus 7 infection in children may be associated with roseola, also known as sixth disease, fever, rash, irritability, mild diarrhea, decreased appetite, swollen eyelids.

Human herpesvirus 8 has been found associated with Kaposi's sarcoma in HIV patients and intra-abdominal tumors.

In humans, B virus causes an encephalitis that is usually fatal. Survivors may suffer brain damage. The reservoir for the disease is latent infection in rhesus monkeys, particularly from Southeast Asia and India. In stressed or unhealth animals, the virus may reactivate and appear in saliva.

Rapid diagnosis of human herpesviruses primary infections or reactivations may include quantitative polymerase chain rection (qPCR) testing. qPCR assays permit simultaneous detection of herpes simplex virus (HSV), varicella zoster virus (VZV), cytomegalovirus (CMV), Epstein-Barr virus (EBV), and human herpesvirus 6 (HHV6).

### Compositions

Antiviral supplement compositions are provided herein for or treating, preventing, and/or preventing reactivation of herpesvirus infections.

Antiviral supplement compositions are provided for treating, preventing, reducing duration of symptoms, reducing severity of symptoms, and reducing likelihood of transmission of a herpesvirus infection, the composition comprising a lysine, an ascorbic compound, a flavonoid glycoside, a pyridoxine, a threonine, and taurine.

Compositions are provided comprising L-lysine hydrochloride, ascorbic acid, rutin, hesperidin, and threonine. The composition may further comprise a pyridoxine. The composition may further comprise a taurine. Compositions are provided comprising L-lysine hydrochloride, ascorbic acid, rutin, hesperidin, a pyridoxine, taurine, and threonine. The composition may further comprise niacinamide ascorbate. The composition may further comprise calcium ascorbate. The composition may further comprise ascorbyl palmitate. In some cases, the composition comprises L-lysine monohydrochloride, ascorbic acid, calcium ascorbate, niacinamide ascorbate, and ascorbyl palmitate hesperidin, rutin, pyridoxine hydrochloride, threonine, and taurine.

L-Lysine is one of the essential amino acids found in proteins. It is needed for proper growth in infants and for maintenance of nitrogen balance in adults. L-lysine is metabolized in mammals to give acetyl-CoA by an initial transamination with alpha-ketoglutarate. L-Lysine is essential for protein synthesis in the body. L-lysine plays a role in calcium absorption, building muscle protein, recovery from injury, and the body's production of hormones, enzymes and antibodies. L-lysine is sometimes used to treat mouth and genital lesions caused by herpes simplex virus as well as shingles caused by herpes zoster viruses. Taking lysine supplements may speed recovery time and reduce the chance of recurrent breakouts of the herpesvirus infection. Lysine supplements are considered generally safe and non-toxic. Griffith et al. Success of L-lysine therapy in frequently recurrent herpes simplex infection. Treatment and prophylaxis. Dermatologica. 1987; 175(4):183-190. Proteins of the herpes simplex virus are rich in L-arginine and tissue culture studies have shown that when the ratio of L-lysine to L-arginine is high, viral replication and cytopathogenicity of the herpes simplex virus may be inhibited.

The antiviral compositions of the present disclosure may comprise a lysine. The term lysine as used herein refers to any pharmaceutically acceptable form including a salt of L-lysine which includes lysine hydrochloride, lysine dihydrochloride, lysine succinate, lysine glutamate, lysine orotate as well as L-lysine. Other acceptable forms of lysine include lysine derivatives such as lysine acetate. In one aspect, the composition comprises from about 40% to about 80% by weight, about 50% to about 70% by weight, or about 50% to 60% by weight of L-lysine hydrochloride, or equivalent. In a specific aspect, the composition comprises about 60% by weight of L-lysine hydrochloride. In one aspect, the compositions and dosage forms of the disclosure comprise an L-lysine or pharmaceutically acceptable salt thereof in single dose range of from about 1,000 mg to about 5,000 mg, 1,000 mg to 4,000 mg, 1,250 to 3,500 mg, 2,000 mg to 3,500 mg, or 2,000 to 3,000 mg.

Vitamin C, also known as ascorbic acid, supports the function of the human immune system. Vitamin C is thought to stimulate the human immune system by enhancing interferon synthesis and lymphocyte activity, particularly the class of lymphocyte referred to as natural killer (NK) cells. Sigel, B. V. & Morton, J. I. “Vitamin C and Immunity: Natural Killer (NK) cell factor” Int. J. Vitamin & Nutrition Res. 1983, 53:179-183; Lovzova, E., Savary, C. A., & Heberman, R. B. “Induction of NK cells activity against fresh human leukemia in culture with interleukin 2” J. Immunology 1987, 138:2718-2727. Natural killer cells are lymphocytes that spontaneously kill tumor or virus-infected cells. Decreased numbers of circulating NK cells have been linked to the development and progression of various immunodeficiencies, viral infections, AIDS, and cancer. Vitamin C is also believed to provide further benefits by acting as an antioxidant aiding the immune system by reducing the amount of free radical damage that can occur as a result of normal body metabolism as well as from exogenous sources.

Studies have shown that vitamin C supplementation (e.g., 500 mg/day) increases plasma concentrations of glutathione. Johnston et al., Am. J. Clin. Nutr. 1993, 58:103-105. Certain data suggest that the thiol antioxidant glutathione (GSH) has an anti-herpesvirus activity. Oxidative stress or other conditions that deplete GSH in the epithelium of the oral, nasal, and upper airway may, therefore, enhance susceptibility to herpesvirus infections. Palamara et al., Antiviral Res. 1995 Jun. 27(3): 237-253.

The antiviral compositions may comprise vitamin C as one or more forms of ascorbic acid, dehydroascorbic acid, ascorbyl ester, or ascorbate salt; together termed ascorbic compounds. In one aspect, the one or more ascorbic compounds are selected from any biologically acceptable form of an ascorbic acid, ascorbyl ester or ascorbate including either or both water-soluble and fat-soluble forms. In some aspects, the one or more ascorbic compounds include any biologically acceptable form that can cross the blood brain barrier, for example, dehydroascorbic acid. Huang et al., 2001, PNAS, vol. 98, no. 20, 11720-11724. The water-soluble form of ascorbic acid can be selected from the group consisting of ascorbic acid, dehydroascorbic acid, a biologically acceptable mono or divalent metal ion salt of ascorbic acid and niacinamide ascorbate, and mixtures thereof. Suitable metal ion salts of ascorbic acid are those selected from the group consisting of calcium ascorbate; magnesium ascorbate; potassium ascorbate; and sodium ascorbate, either alone or some mixture thereof. Other water soluble forms can include manganese ascorbate; zinc ascorbate; iron ascorbate; copper ascorbate; boron ascorbate; molybdenum ascorbate; and chromium ascorbate. The fat soluble ascorbyl esters preferably comprise fatty acid esters of saturated or unsaturated carboxylic acids with ascorbyl palmitate being one preferred form. Other fat soluble esters of ascorbic acid which are preferred include: ascorbyl palmitate; ascorbyl arachidonate; ascorbyl stearate; ascorbyl linoleate; ascorbyl linoleneate; and ascorbyl oleate.

The antiviral compositions may comprise from about 10% to about 40% by weight, about 15% to about 35% by weight, or about 20% to about 30% by weight combined of one or more ascorbic compounds, or equivalent. In a specific aspect, the composition comprises about 25% by weight of combined ascorbic acid, calcium ascorbate, niacinamide ascorbate, and ascorbyl palmitate. In another aspect, the composition comprises about 0.5 to about 2.0 g, about 0.5 g to about 1.5 g combined weight of one or more ascorbic compounds per dose. In one aspect, certain compositions comprise about 0.1 g to about 1.5 g, or about 0.1 g to 0.3 g, ascorbic acid per dose. In another aspect, certain compositions of the disclosure may comprise from about 0.2 g to about 0.75 g, or about 0.25 g to about 0.6 g calcium ascorbate per dose. In a further aspect, certain compositions of the disclosure comprise from about 0.1 g to about 0.5 g, or about 0.1 g to about 0.3 g, niacinamide ascorbate per dose. In another aspect, certain compositions of the disclosure comprise from about 0.01 g to about 0.1 g, or about 0.02 g to about 0.05 g, ascorbyl palmitate per dose.

Flavonoids are a large family of compounds synthesized by plants that have a common chemical structure. Flavonoids are further divided into subclasses based upon chemical structure. Flavonoids are found in fruits and vegetables in the diet. Flavonoids connected to one or more sugar molecules are known as flavonoid glycosides, while those that are not connected to a sugar molecule are called aglycones. With the exception of flavanols (catechins and proanthocyanidins), flavonoids occur in plants and most foods as glycosides. Even after cooking, most flavonoid glycosides reach the small intestine intact. Only flavonoid aglycones and flavonoid glucosides (bound to glucose) are absorbed in the small intestine, where they are rapidly metabolized to form methylated, glucuronidated, or sulfated metabolites. The ability of flavonoids to chelate (bind) metal ions appears to contribute to their antioxidant activity in vitro. Although it was initially hypothesized that the biological effects of flavonoids would be related to their antioxidant activity, available evidence from cell culture experiments suggests that many of the biological effects of flavonoids are related to their ability to modulate cell-signaling pathways (Williams et al., Flavonoids: antioxidants or signalling molecules?Free Radic. Biol. Med.; 36(7):838-849 (2004)).

The antiviral compositions may comprise one or more flavonoid glycosides. The flavonoid glycosides may include any flavonoid which is glycosylated. In a specific aspect, the compositions comprise one or more flavonoid glycosides selected from hesperidin, rutin, naringin, and quercitrin, or a pharmacologically acceptable salt thereof. The rutin can be rutin of a pharmacologically acceptable salt thereof. The rutin can be rutin. The rutin can be sodium rutin. The hesperidin can be hesperidin or a pharmacologically acceptable salt thereof. The hesperidin can be hesperidin. The hesperidin can be hesperidin. In one specific aspect, the compositions comprise hesperidin and rutin.

The antiviral composition may comprise from about 5% to about 25% by weight, preferably about 10% to about 15% by weight combined of one or more flavonoid glycosides, or equivalent. In a specific aspect, the composition comprises about 10-15% by weight of combined hesperidin complex and/or rutin. In some cases, the compositions comprise about from about 0.1 g to about 0.8 g, or about 0.1 g to about 0.3 g, hesperidin per single dose. In another aspect, the compositions comprise from about 0.1 to about 0.5 g, or about 0.1 g to about 0.3 g, rutin per single dose.

Threonine is an essential amino acid that is a necessary building block for protein. It promotes the growth of the thymus, a small gland that regulates many of the hormones and cells vital to immune defense. Even a moderate reduction in dietary intake of threonine can produce a depression in immune response or antibody production. Lotan. Humoral and cellular immune response in growing rats fed a 10% gluten diet. Isr. J. Med. Sci. 1989 August; 25(8):437-41. Recent research suggests that the effects of threonine relate to a specific requirement by the thymus for this amino acid and its ability to promote cell immune defense functions. Braverman, Threonine: The Immunity Booster, The Healing Nutrients Within, 2003; 13 pp:201.

The antiviral compositions may comprise a threonine. The threonine can be selected from L-threonine or any pharmaceutically acceptable salt or derivative thereof. The threonine may be L-threonine or pharmaceutically acceptable salt thereof, for example, L-threonine, L-threonine potassium salt, or L-threonine sodium salt. The antiviral compositions may comprise from about 0.1% to about 5% by weight, about 0.5% to about 2% by weight, or about 0.75-1.5% by weight, of L-threonine, or equivalent. In a specific aspect, the composition comprises about 1% by weight of L-threonine. In one aspect, the compositions of the disclosure comprise from about 0.01 to about 0.08 g, or about 0.02 to about 0.05 g L-threonine per dose.

The antiviral compositions may comprise a pyridoxine. In some cases, the pyridoxine is selected from the group consisting of pyridoxine, pyridoxine hydrochloride, pyridoxine phosphate, pyridoxal, pyridoxal hydrochloride, pyridoxal 5-phosphate, pyridoxic acid, pyridoxamine, pyridoxamine hydrochloride, and pyridoxamine dihydrochloride. Pyridoxine is one form of vitamin B6. Pyridoxine is utilized by the liver to synthesize pyridoxal phosphate (PLP), the active coenzyme form. PLP is a cofactor for the enzyme threonine aldolase which catalyzes the conversion of hydroxy-N-trimethyl-L-lysine to trimethylaminobutyraldehyde; intermediates in the conversion of L-lysine to L-carnitine. Pyridoxine deficiency is known to produce a significant reduction in plasma carnitine levels. Absorption and Utilization of Amino Acids, Vol. II, Mendel Friedman, CRC Press, 1989, ISBN 0849360072, Ch. III, p. 48-49. Pyridoxine is a water soluble B vitamin which serves as a cofactor and is involved in the metabolism of protein, carbohydrates, and the production of insulin and red and white blood cells. Vitamin B6 is essential in numerous biochemical pathways in the immune system. Pyridoxine deficiency leads to impairment of immune responses (Trakatellis et al., 1997, Pyridoxine deficiency: new approaches in immunosuppression and chemotherapy. Postgrad. Med. J. October; 73(864): 617-622 (1997)). Vitamin B6 is usually safe at intakes up to 200 mg per day in adults. In some cases, the pyridoxine is pyridoxal. In some cases, the pyridoxine is pyridoxal phosphate. In some cases, the pyridoxine is pyridoxal 5-phosphate. In some cases, the pyridoxine is pyridoxine hydrochloride. In some cases, the pyridoxine is pyridoxal hydrochloride. In some cases, the pyridoxine is pyridoxamine. In some cases, the pyridoxine is pyridoxic acid. In some cases, the composition may comprise from about 0.1% to about 2% by weight, about 0.5 to about 2% by weight, or about 0.5% to about 1.5% by weight of the pyridoxine, or about 1% by weight of the pyridoxine. In some cases, antiviral compositions of the disclosure may comprise from about 0.02 g to about 0.08 g, or about 0.02 to about 0.04 g, or about 0.04 g to about 0.08 g of the pyridoxine per dose.

The antiviral compositions may comprise taurine. Taurine, or 2-aminoethanesulfonic acid, is a metabolite of the sulfur-containing amino acid, cysteine. Taurine is one of the few known naturally occurring sulfonic acids. Metabolic actions of taurine include bile acid conjugation, detoxification, membrane stabilization, osmoregulation, and modulation of cellular calcium levels. Taurine is able to cross the blood brain barrier. Taurine acts as an antioxidant and protects against toxicity of various substances (Green et al., Antioxidant role and subcellular location of hypotaurine and taurine in human neutrophils. Biochimica et biophysica acta, January 23; 1073(1):91-7 (1991)). Taurine is also known to play a role in the immune system. For example, taurine interacts with hypochlorous acid, produced during the “oxidant burst” of stimulated macrophages, to produce taurine chloramine (TauCl). This compound may have important immunomodulatory properties and may be responsible for properties that have been ascribed earlier to taurine. In vitro studies have shown that an increase in taurine concentration from physiological to superphysiological concentrations has no effect on proinflammatory cytokine production by peripheral blood mononuclear cells; however, Tau-Cl modulates synthesis of pro-inflammatory cytokines, and therefore it may play a role in the initiation and propagation of immune response (Chorazy et al., Taurine chloramine modulates cytokine production by human peripheral blood mononuclear cells. Amino Acids. 23:407-13 (2002)). Tau-Cl inhibits nuclear factor κB activation and the capacity for proinflammatory cytokine production, producing an anti-inflammatory effect (Huxtable RJ., Taurine past, present, and future. Adv Exp Med Biol. 403:641-50 (1996)). Taurine in 0.9 to 1.4 grams per day have been tolerated without documented adverse effects (Braverman, Taurine: The Seizure Fighter, The Healing Nutrients Within, Basic Health Publications, Inc., Laguna Beach, California, Ch. 8, pp: 132-133 (2003)).

The antiviral compositions may comprise a taurine, or pharmacologically acceptable salt or derivative thereof. In one aspect, the composition comprises from about 1% to about 10% by weight, about 2% to about 6% by weight, or about 3% to about 5% by weight, of taurine, or equivalent. The antiviral composition may comprise about 4% by weight of taurine. Antiviral compositions may comprise from about from about 0.02 g to about 0.4 g, or about 0.05 g to about 0.2 g, taurine per dose.

Antiviral compositions formulated for oral administration are provided. The oral composition may be in the form of a powder, capsule, tablet, lozenge, troche, soft chew, gummy, orally dissolvable tablet, liquid, or caplet. The powder may be utilized in a capsule fill, sold in a single dose packet meant to mix with a food such as applesauce, or can be an effervescent powder formulation sold in a single dose packet and meant for suspension in a liquid. The capsule, tablet, may be ingested by swallowing. In another aspect, the tablet, capsule, lozenge, troche, soft chew, gummy, orally dissolvable tablet may be orally disintegrable. In some cases, the tablet, capsule, lozenge, troche, soft chew, gummy, orally dissolvable tablet may be formulated in a slow-release composition. In some cases, the capsule, tablet, may be ingested by swallowing. In another aspect, the tablet, capsule, lozenge, troche, soft chew, gummy, orally dissolvable tablet may be an immediate-release composition. In some cases, the oral composition may be formulated as a prepackaged liquid drink, wherein the formulation is suspended in a flavored liquid. In some cases, the composition is in the form of a tablet, a capsule, or a powder meant to mix with a food, such as applesauce. Forms for other modes of administration may include parenteral forms, or anal suppositories.

Active ingredients of representative anti-herpesvirus compositions are shown in Table 1.

TABLE 1
Anti-Herpesvirus Compositions Active Ingredients

| | | | Range, | Range, |
|---|---|---|---|---|
| Active Ingredient | range, g | range, g | wt % | wt % |
| L-Lysine hydrochloride | 1.0-5.0 | 2.0-3.5 | 40-80 | 50-60 |
| Ascorbic compound(s) | 0.1-1.5 | 0.5-1.3 | 2-30 | 5-10 |
| Flavonoid glycoside(s) | 0.3-1.5 | 0.5-0.8 | 5-25 | 10-15 |
| L-Threonine | 0.01-0.08 | 0.02 -0.06 | 0.1-2.0 | 0.3-1.2 |
| Pyridoxine HCl or a | 0-0.1 | 0.04-0.08 | 0-3 | 0.5-2 |
| pyridoxal | | | | |
| Taurine | 0-0.4 | 0.02-0.3 | 0-8 | 2-6 |
| Total active weight per | 1.4-8.5 g | 3-6 g | 100% | 100% |
| dose (g) | | | | |

In some cases, a single oral dose of the composition may comprise from about 1.2 g to about 3.5 g L-lysine monohydrochloride; from about 0.1 g to about 1.5 g ascorbic acid or dehydroascorbic acid; from about 0.1 g to about 0.8 g hesperidin; from about 0.1 g to about 0.5 g rutin; and from about 0.01 g to about 0.08 g threonine. The composition may further comprise from about 0.02 g to about 0.4 g taurine. The composition may further comprise from about 0.02 g to about 0.08 g a pyridoxine, such as pyridoxine hydrochloride or a pyridoxal. The composition may further comprise from about 0.5 g to about 0.75 g calcium ascorbate; from about 0.1 g to about 0.5 g niacinamide ascorbate; and from about 0.01 g to about 0.1 g ascorbyl palmitate. In some cases, a single dose of the composition comprises about 1.2 to about 3 g L-lysine monohydrochloride; from about 0.2 g to about 1.0 g ascorbic acid or dehydroascorbic acid; about 0.1 to about 0.3 g hesperidin; about 0.1 to about 0.3 g rutin; and about 0.02 to about 0.05 g threonine. In some cases, the composition may further comprise about 0.02-0.05 g pyridoxine hydrochloride. In some cases, the composition may further comprise from about 0.02 g to about 0.3 g taurine. In some cases, a single dose of the composition further comprises about 0.6 g calcium ascorbate; 0.3 g niacinamide ascorbate, and about 0.05 g ascorbyl palmitate. In some cases, the ascorbic compound is selected from one or more, two or more, or three or more of ascorbic acid, calcium ascorbate, niacinamide ascorbate, and ascorbyl palmitate. In some cases, the ascorbic compound is a mixture of ascorbic acid, calcium ascorbate, niacinamide ascorbate, and ascorbyl palmitate. In some cases, the ascorbic compound comprises dehydroascorbic acid. In some cases, the composition comprises 50-60 wt % of L-lysine HCl, 3-7 wt % ascorbic acid or dehydroascorbic acid, 10-15 wt % calcium ascorbate dihydrate, 4-8 wt % niacinamide ascorbate, 0.5-2 wt % ascorbyl palmitate, 4-8 wt % hesperidin, 4-8 wt % rutin, 0.5-2 wt % L-threonine, optionally 2-6 wt % taurine, and optionally 0.5-2 wt % pyridoxine HCl.

The compositions and various dosage forms of the disclosure may comprise, aside from those active ingredients specified above, other various additives, such as vehicle, binder, disintegrating agent, lubricant, thickener, surfactant, osmotic pressure regulator, electrolyte, sweetener, flavoring, perfume, pigment, pH regulator and others appropriately as required.

Specifically, the additives may include starches such as wheat starch, potato starch, corn starch, and dextrin, sugars such as sucrose, glucose, fructose, maltose, xylose, and lactose, sugar alcohols such as sorbitol, mannitol, maltitol, and xylitol, isotransposable glycosides such as coupling sugar and paratinose, vehicles such as calcium phosphate and calcium sulfate, binders and thickeners such as starch, sugar, gelatin, gum arabic, dextrin, methyl cellulose, polyvinyl pyrrolidone, polyvinyl alcohol, hydroxy propyl cellulose, xanthan gum, pectin, tragacanth gum, casein, and alginic acid, lubricants such as leucine, isoleucine, valine, sugar-ester, hardening oil, stearic acid, magnesium stearate, talc, and macrogol, disintegrating agents such as avicel, carboxymethyl cellulose (CMC), CMC-Na, CMC-Ca, silicon dioxide, fillers or diluents such as calcium carbonate, mannitol, surfactants such as polysorbate and lecithin, and sweeteners such as sugars, sugar alcohols, aspartame, alitame, other dipeptides, stevia, and saccharin, and they may be used in proper amounts selectively in consideration of the relation with the essential components, property of the composition, manufacturing method, etc.

The compositions of the disclosure may optionally further comprise one or more flavoring agents. The optional flavoring agent is added to increase patient acceptability and compliance with the recommended dosing schedule. The flavoring agents that may be used include those flavors known to the skilled artisan, such as natural and artificial flavors. These flavorings may be chosen from synthetic flavor oils and flavoring aromatics and/or oils, oleoresins and extracts derived from plants, leaves, flowers, fruits, and so forth, and combinations thereof. Non-limiting representative flavor oils include spearmint oil, cinnamon oil, oil of wintergreen (methyl salicylate), peppermint oil, clove oil, bay oil, anise oil, eucalyptus oil, thyme oil, cedar leaf oil, oil of nutmeg, allspice, oil of sage, mace, oil of bitter almonds, and cassia oil. Also useful flavorings are artificial, natural and synthetic fruit flavors such as vanilla, and citrus oils including, without limitation, lemon, orange, lime, grapefruit, and fruit essences including apple, berry, pear, peach, grape, strawberry, raspberry, cherry, plum, pineapple, apricot and so forth. These flavoring agents may be used in liquid or solid form and may be used individually or in admixture. Commonly used flavors include mints such as peppermint, menthol, artificial vanilla, cinnamon derivatives, and various fruit flavors, whether employed individually or in admixture. Other useful flavorings include aldehydes and esters such as cinnamyl acetate, cinnamaldehyde, citral diethylacetal, dihydrocarvyl acetate, eugenyl formate, p-methylamisol, and so forth may be used. In a specific aspect, the flavoring is spearmint oil. The flavor is optionally present from about 0.1% to about 5% by weight of the antiviral composition.

Tablets may be molded tablets or compressed tablets. Tablets may be formed by wet granulation, dry granulation, and direct compression. These techniques are known to one of skilled in the art and are described, for example, in the United States Pharmacopeia National Formulary USP XXII, 1990, pp. 1696-1697. Various other vitamins may be added to composition. Tablets may optionally further comprise flavorings or sweeteners. In one aspect, the sweetened, flavored tablet is utilized as a lozenge to be dissolved in the mouth. The compositions of the disclosure can also be prepared in a chewable form or an effervescent form. For effervescent preparations, the manufacturing method in the disclosure is basically same as in the manufacturing method of the usual effervescent preparations such as effervescent tablets. That is, components are weighed, mixed, and prepared directly by the powder compression method, dry or wet granular compression method, etc. Orally disintegrable tablets are described, for example, in U.S. Pat. No. 7,431,942, Shimuzu et al., which is incorporated herein by reference. Lozenges with a hard candy base can be prepared, for example, by the techniques of U.S. Pat. No. 6,316,008, Godfrey, which is incorporated herein by reference.

Liquid compositions are provided. Liquid compositions may further comprise other nutrients. Such liquid compositions may be prepared as described in U.S. Pat. No. 6,037,375, Sakamoto et al., which is incorporated herein by reference. A nutrient liquid composition of the disclosure contains a lysine, an ascorbic compound, a flavonoid glycoside, a threonine and a pyridoxine as essential ingredients, and is prepared in the same manner as the ordinary food and beverage, and other food materials may be appropriately added. As particularly preferred food materials, sweeteners such as organic acids and carbohydrates may be used. Organic acid components include citric acid, tartaric acid, malic acid, and succinic acid, and citric acid is particularly preferable. These organic acids are added usually in a range of 100 to 1500 mg/100 ml, preferably 250 to 800 mg/100 ml, and the composition of the material in beverage form can be prepared.

Various sweeteners can be optionally used in the tablet, liquid, capsule, lozenge or troche formulations of the disclosure. Examples of carbohydrates and sweeteners include monosaccharides such as glucose and fructose, disaccharides such as maltose, sucrose, other ordinary sugars, sugar alcohols such as xylitol, sorbitol, glycerin and erythritol, polysaccharides such as dextrin and cyclodextrin, and oligosaccharides such as fructo-oligosaccharide, galacto-oligosaccharide and lacto-sucrose. Of the carbohydrates, as the components not adversely affecting the lipid metabolism, fructose and glycerin are preferred. As oligosaccharide, addition of lacto-sucrose is preferred. A beverage composition of the disclosure can increase bifidobacteria in the body or lower the putrefaction products depending on the blend of the lacto-sucrose, so that the immune system can be intensified further. Other sweeteners include natural sweeteners such as thaumatin, stevia extract, rebaudioside A, glycyrrhizinic acid, etc. and synthetic sweeteners such as saccharin, aspartame, etc. These carbohydrates may be also added as carbohydrate mixture such as isomerized sugar and refined sugar. The sweetener is optionally present from about 0.1% to about 5% by weight of the solid composition. The blending of the carbohydrates may be about 1 to 15 g in 100 ml of the beverage composition of the disclosure, preferably about 3 to 12 g. The content of the oligosaccharide is about 0.5 to 10 g, preferably 1 to 3 g.

The liquid composition may also comprise, aside from the above, various nutrients, vitamins, minerals (electrolytes) including trace elements, perfumes including synthetic perfumes and natural perfumes, coloring matter, flavors (fruit, vanilla, chocolate, etc.), pectic acid and its salts, alginic acid and its salts, organic acids, thickener as protective colloidal substance, pH regulator, stabilizer, preservative, glycerins, alcohols, and sparkling component for carbonated beverages. In addition, the composition of the disclosure may also contain natural juice or fruit to be presented as fruit drink or vegetable drink. These may be used either alone or in combination of two or more kinds. The blending rate of these additives is not particularly limited, and is generally selected in a range of about 0 to 20 parts by weight to 100 parts by weight of the composition of the disclosure.

The composition may optionally include additional vitamins. Optional additional vitamins include, whether water-soluble or fat-soluble, thiamine, niacin, retinol palmitate, bisbentiamine, riboflavin, cyanocobalamin, cholecalciferol, nicotinic acid amide, calcium pantothenate, folic acid, biotin, and choline ditartate, and those belonging to vitamin B group.

The liquid composition may be prepared by blending these components, and the method of preparation is not particularly limited, and all components may be blended simultaneously, but more preferably fat soluble components are preliminarily dissolved in oil, and water-soluble components in water, then the solution is emulsified by using an emulsifier, so that the composition of the disclosure may be prepared. More preferably, the oil solution is added to water and a proper emulsifier to emulsify, and an aqueous solution is added and blended to the obtained emulsion. The blending operation of the components may be executed under ordinary temperature, or preferably executed by slight heating operation. The emulsification can be executed by using a proper emulsifying machine, for example, homo-mixer or high pressure homogenizer, either by complete passing system or by circulation system. The emulsion after emulsification is filtered by conventional process, and poured into proper containers and sterilized, so that a desired beverage product is obtained. Sterilization may be effected by heating, aseptic filtering, etc.

Liquid compositions such as a carbonated beverages may be prepared including injecting carbon dioxide may be injected into the emulsifier by conventional process. Such beverage is preferred to be prepared in the osmotic pressure range of about 260 to 600 mOsm/kg.

The composition of the disclosure may be also prepared in an effervescent form. The effervescent form may contain, aside from the active ingredients of the disclosure, proper amounts of sodium carbonate and/or sodium hydrogen carbonate and neutralizing agent as foaming components. The neutralizing agent used herein is an acidic compound capable of generating carbon dioxide by neutralizing sodium carbonate or sodium hydrogen carbonate. Such compound includes, for example, L-tartaric acid, citric acid, fumaric acid, ascorbic acid and other organic acid. Preferred ascorbic acid possesses both the action of neutralizing agent and the action of antioxidant.

### Topical Compositions

The disclosure provides topical compositions comprising a therapeutically effective amount of a composition comprising a lysine, an ascorbic compound, a flavonoid glycoside, a threonine, optionally a taurine, and optionally a pyridoxine.

A topical composition is provided for use in transdermal or transmucosal administration. The topical composition may be in the form of an ointment, lip balm, cream, paste, gel, lotion, emulsion, or suspension. The topical composition may include one or more pharmaceutically acceptable excipients. The pharmaceutically acceptable excipients may include vehicles or bases, binders, emulsifiers, preservatives, pH adjusters, suspending agents, solubilizing agents, solvents, lubricants, penetration enhancers, colorants, flavorings, and/or anesthetic or anti-inflammatory.

In some cases, the topical composition includes a vehicle and/or base. The examples of vehicles or bases may include but not limited to hydrocarbons, waxes, silicones, alcohols, sterols, sterol esters, lanolin, anhydrous lanolin, lauric acid, myristic acid, palmitic acid, stearic acid, oleic acid, polyols, polyglycols, bees wax, white bees wax (bleached bees wax), carnauba wax, myricin, cholesterol esters (stearate), polyoxyethylene sorbitain monoesters (stearate-tweens), lard, aloe vera, aloe barbadensis leaf juice, grapeseed oil, olive oil, hydrogenated oils, or mixture thereof.

In some cases, the topical composition includes a binder. The examples of binders may include but are not limited to polyvinyl pyrrolidone, polyethylene glycol, carboxymethyl cellulose, hydroxypropyl methylcellulose (HPMC), hydroxypropyl cellulose, gum acacia, ethyl cellulose, starch, gelatin or mixtures thereof.

In some cases, the topical composition includes an emulsifier. The examples of emulsifier may include but not limited to cholesterol (cholesterin), lanolin (hydrous wool fat, lanum), anhydrous lanolin (wool fat, anhydrous lanum, agnin), glycerol stearate, semi synthetic lanolins, sodium, potassium ethanolamin salts of lauric acid, myristic acid, palmitic acid, stearic acid, oleic acid, polyethylene-polypropylene glycols (pluronics), polysorbates (Tweens), sodium lauryl sulfate, borax (sodium borate), ethanolamine, triethanolamine or mixture thereof.

In some cases, the topical composition includes a preservative. The examples of preservatives may include but not limited to antimicrobial agent such as benzalkonium chloride, benzoic acid, phenoxyethanol, benzyl alcohol, bronopol, chlorhexidine, chlorocresol, imidazolidinyl urea, paraben esters, phenol, disodium EDTA, phenoxyethanol, potassium sorbate, sorbic acid or mixture thereof and antioxidants such as a-tocopherol, ascorbic acid, ascorbyl palmitate, butylated hydroxyanisole, sodium ascorbate, sodium metabisulfite, or mixture thereof.

In some cases, the topical composition includes a pH adjuster. The examples of pH adjuster may include but are not limited to fumaric acid, sodium bicarbonate, citric acid, sodium hydroxide, triethanolamine, borate buffer, sodium monophosphate, disodium phosphate, or mixture thereof.

In some cases, the topical composition includes a suspending agent, gelling agent, and/or thickener. The examples of suspending agents, gelling agents, and or thickeners may include but not limited to fumed silica (cab-O-sil), bentonite (colloidal aluminum silicate), veegum (colloidal magnesium aluminum silicate), agar, alginates, carragen, acacia, tragacanth, methyl cellulose, carboxymethyl cellulose, hydroxyethyl cellulose, carboxy vinyl polymer, copolymers of acrylic acid with allyl sucrose or allyl ethers of pentaerythritol (Carbomers), gelatin, pectin, xanthan, polyacrylic acid or mixture thereof.

In some cases, the topical composition includes a solubilizing agent. The examples of solubilizing agent may include but are not limited to polyethylene glycol, cyclodextrin or its salts and surfactants or combination thereof, wherein surfactants may include polysorbates (Tween), spans, sodium laurylsulfate (SLS); acyl sulfates such as dioctyl sodium sulfosuccinate (DOSS) or mixture thereof.

In some cases, the topical composition includes a solvent. The examples of solvents may include but are not limited to aqueous and non-aqueous solvents. The non-aqueous solvent may include but are not limited to acetone, methanol, ethanol, isopropyl alcohol, polyethylene glycol, propylene glycol or mixture(s) thereof.

In some cases, the topical composition includes a lubricant. The examples of lubricants may include but are not limited to talc, fats, cetyl alcohol, magnesium stearate, or stearic acid or mixture thereof.

In some cases, the topical composition includes an anti-inflammatory agent. In some cases, the anti-inflammatory agent is selected from the group consisting of ibuprofen, ketoprofen, felbinac, piroxicam, salicylate, diclofenac, diclofenac sodium, diclofenac epolamine, capsaicin, menthol, eltenac, etoricoxib, felbinac, flufenamate, indomethacin, and the like.

In some cases, the topical composition includes an anesthetic. In some cases, the anesthetic is selected from the group consisting of lidocaine, epinephrine, norepinephrine, phenylephrine, lignocaine, prilocaine, tetracaine, bupivanor, benzocaine, proparacaine, bupivacaine, and the like.

In some cases, the topical composition includes a penetration enhancer. The penetration enhancer may be any appropriate penetration enhancer. For example, Pereira et al., 2021, Membranes 11, 343 disclose various penetration enhancers including amino acid-based permeation enhancers in transdermal drug delivery. Permeation enhancers include solvents such as ethanol, propylene glycol, glycerol, DMSO, and N-methylpyrrolidone; fatty acids and alcohols such as oleic acid, docecanol; lactams such as N-dodecylpyrrolidone and N-dodecylazenpan-2-one (Azone); terpenes such as menthol, limonene, and farnesol; sugar and vitamin derivatives such as sorbitan monooleate, ascorbyl palmitate, and 2-deoxy-D-glucose; esters such as isopropyl myristate, glycerol monolaurate, and ethyl oleate; and amino acids and amino acid derivatives such as dodecyl N-acetylproline (Pro2), transkarbam 12, dodecyl 2-dimethylaminopropionate (DDAIP), 6-(dimethylamino) hexanoic acid, L-Proline, beta-alanine, serine, glycine-histidine, L-Leucine, and N-dodecanoyl-amino acid methyl esters of glycine, serine, valine, alanine, tryptophan, leucine, phenylalanine, tyrosine, and methionine.

In some cases, the topical composition includes a colorant. The examples of colorants may include but are not limited to titanium dioxide, iron oxide (e.g. iron oxide yellow, iron oxide red, iron oxide brown, iron oxide black) or mixtures thereof.

A topical gel can be prepared from a composition according to the disclosure. For example, a topical gel may be prepared comprising 10-30 wt % lysine monohydrochloride, 2-15 wt % of one or more ascorbic compounds, 2-10 wt % flavonoid glycosides, 0.05-1 wt % L-threonine, 0-1.5 wt % pyridoxine HCl, and 0-4 wt % taurine. In one example, the lysine monohydrochloride, water-soluble ascorbic compounds, L-threonine, optional pyridoxine HCl, and optional taurine are dissolved in minimal purified water (q.s.) to create a solution. The flavonoid glycosides are dissolved in polyethylene glycol (10 wt %) which is added to the solution with polysorbate 80 (5 wt %). Carbomer 974P (0.5-2 wt %) aqueous gel is formed and benzyl alcohol (2 wt %) and Pemulen™ polymeric emulsifier (Lubrizol Pharmaceuticals, 1 wt %, copolymers of acrylic acid and C10-C30 alkyl acrylate crosslinked with allyl pentaerythritol, Carbomer copolymer) is added. The solution is added to the Carbopol gel and filled to a collapsible tube.

In some cases, the topical composition comprises Aloe barbadensis leaf juice, cetyl alcohol, disodium phosphate, disodium EDTA, glycerol stearate, Phenoxyethanol, water, sodium hydroxide.

The compositions may be formulated as oral compositions. Compositions may be provided in the form of a powder, capsule, tablet, orally dissolving tablet, gummy, lozenge, troche, liquid, or caplet.

### Administration

Compositions of the disclosure may be administered by an oral administration. Generally, a therapeutically effective dose is not less than about 10% and not more than about 200% of the amounts of individual ingredients listed in Table 1. In certain aspects, a therapeutically effective dose for treatment of a viral infection is from about 50% to about 150%, or from about 80% to about 120%, or about 100% identical with the list of components in Table 1. In another aspect, a therapeutically effective dose for treatment of a viral infection in a subject in need thereof is administered every 4 to 6 waking hours, or from about two to six times per day. In a further aspect, a therapeutically effective dose for prophylaxis of a viral infection in a subject in need thereof may be administered every 8 to 12 waking hours, or from about one to three times per day.

### Methods of Treating

Methods and compositions are provided for shortening healing during first outbreak, lowering frequency of recurrent outbreaks, lessening severity and duration of symptoms in recurrent outbreaks, and reducing chance of passing herpesvirus to a partner.

A method of reducing herpesvirus replication in a cell is provided comprising treating a virus-infected cell with an effective amount of a composition comprising a lysine, an ascorbic compound, a flavonoid glycoside, a threonine, a taurine, and a pyridoxine. The lysine may be selected from L-lysine, L-lysine monohydrochloride, L-lysine dihydrochloride, L-lysine succinate, L-lysine glutamate, and L-lysine orotate. The ascorbic compound may be selected from one or more of ascorbic acid, calcium ascorbate, magnesium ascorbate, potassium ascorbate, sodium ascorbate, manganese ascorbate, zinc ascorbate, iron ascorbate, copper ascorbate, boron ascorbate, molybdenum ascorbate, chromium ascorbate, ascorbyl palmitate, ascorbyl arachidonate, ascorbyl stearate, ascorbyl linoleate, ascorbyl linoleneate, and ascorbyl oleate. The flavonoid glycoside may be selected from one or more of hesperidin, rutin, naringin, and quercitrin. The pyridoxine may be pyridoxine hydrochloride.

A method of reducing herpesvirus replication in a cell is provided comprising treating a virus-infected cell with an effective amount of a composition comprising L-lysine monohydrochloride, ascorbic acid, calcium ascorbate, niacinamide ascorbate, ascorbyl palmitate, hesperidin, rutin, pyridoxine hydrochloride, threonine, and taurine.

A method is provided for the treatment or prevention, reducing the frequency of recurrence, or reducing duration of an outbreak, of a herpesvirus infection in a subject in need thereof comprising administering to the subject a therapeutically effective amount of a composition according to the present disclosure. In some cases, the anti-herpesvirus composition comprises a lysine, an ascorbic compound, a flavonoid glycoside, a threonine, a taurine, and a pyridoxine.

The herpesvirus may be selected from the group consisting of herpes simplex type 1 (HSV-1), herpes simplex type 2 (HSV-2), varicella zoster virus (VZV), Epstein-Barr virus (EBV), human cytomegalovirus (HCMV), human herpesvirus 6A (HHV-6A), human herpesvirus 6B (HHV-6B), human herpesvirus 7 (HHV-7), and human herpesvirus 8 (HHV-8). In some cases, the herpesvirus may be selected from the group consisting of HSV-1 and HSV-2.

### EXAMPLES

### Example 1. Antiviral Compositions

### Example 1A. Powder Formulations

Ingredients for various test formulations are shown in Table 2; the amounts for a single dose packet of powder are shown. A test batch for each formulation was prepared utilizing five times the amounts shown. Each ingredient was added to the batch and the batch was mixed thoroughly, divided by weight and sealed into five packets. Alternatively, the powder can be filled to hard shell capsules.

TABLE 2
Test Formulations- weight per dose for powder formulation.

| | Formula | Formula | Formula | Formula | Formula | Formula |
|---|---|---|---|---|---|---|
| Ingredient | 1 | 2 | 3 | 4 | 5 | 6 |
| L-Lysine HCl | 3.00 | 3.00 | 3.00 | 3.00 | 3.00 | 3.00 |
| L-Threonine | 0.05 | 0.06 | 0.05 | 0.05 | 0.05 | 0.05 |
| Ascorbic acid | 0.28 | 1.00 | 0.45 | 0.28 | 0.28 | 0.28 |
| Calcium | 0.64 | — | 0.64 | 0.64 | 0.64 | 0.64 |
| ascorbate, | | | | | | |
| dihydrate | | | | | | |
| Niacinamide | 0.30 | — | — | 0.30 | 0.30 | 0.30 |
| ascorbate | | | | | | |
| Ascorbyl | 0.05 | — | 0.05 | 0.08 | 0.08 | 0.08 |
| palmitate | | | | | | |
| Hesperidin | 0.30 | 0.33 | 0.30 | 0.50 | 0.50 | 0.50 |
| complex | | | | | | |
| Rutin NF | 0.30 | 0.33 | 0.30 | 0.50 | 0.50 | 0.50 |
| Pyridoxine HCl | 0.05 | 0.05 | 0.05 | 0.05 | 0.05 | — |
| Taurine | 0.20 | 0.04 | 0.20 | 0.20 | — | — |
| Total weight | 5.17 | 4.81 | 5.04 | 5.60 | 5.40 | 5.35 |
| per dose (g) | | | | | | |

### Example 1B. Tablet Formulations

Tablet formulations were prepared using the ingredients shown in Table 2. The amount of ingredient shown is equal to one dose. Five tablets contained one dose of the formulation. A wet granulation technique was employed and compressed tablets were coated with a white coating shown in Table 3. Tablets were dried and stored at room temperature.

TABLE 3
Tablet Formulations- amount per tablet.

| | Formula | Formula |
|---|---|---|
| Ingredient | FT (mgs) | VT (mgs) |
| Citrus Bioflavonoid (Hesperidin) | 142.97 | 66.60 |
| 98% | | |
| Methocel E-5 HPMC | 4.42 | 5.00 |
| L-Lysine Monohydrate | 612.20 | 600.00 |
| Ascorbic Acid | 218.80 | 200.00 |
| Rutin NF | 30.00 | 66.00 |
| Pectin | 30.00 | 66.00 |
| Pyridoxine HCl USP Vit B6 | 13.40 | 10.00 |
| Taurine | 8.20 | 8.00 |
| L-Threonine | 12.20 | 12.00 |
| Microcrystalline Cellulose | 200.00 | 200.00 |
| Croscarmellose Sodium | 20.00 | 20.00 |
| Silica | 6.00 | 6.00 |
| Magnesium Stearate | 10.00 | 10.00 |
| Riboflavin Vit B2 | 1.50 | 0 |
| Purified Water | | |
| White Coating including: | 40.00 | 40.00 |
| Dextrin | | |
| Dextrose | | |
| Hypromellose | | |
| Mineral Oil | | |
| Polyethylene Glycol | | |
| Polysorbate 80 | | |
| Croscarmellose Sodium | | |
| Sodium Citrate | | |
| Titanium Dioxide | | |
| Total Mgs: | 1349.69 | 1308.82 |

### Example 1C. Orally Dissolving Tablet Formulations

The composition of Formula 1 was mixed with mannitol, calcium carbonate, citric acid, natural flavor, silicon dioxide, stevia rebaudioside A, magnesium stearate, and sucralose and compressed into tablets. The single dose amounts shown in table 2 were divided into four tablets.

### Example 2. pH Study

Cell based studies were considered for testing antiviral effects of test compositions. To understand pH effects of aqueous test compositions that might alter cell toxicity, pH of test compositions in DMEM and two different buffered DMEM media was performed to select medium for testing purposes.

### Preparation of Test Media:

Commercial Dulbecco's Modified Eagle's Medium (DMEM) (10-013-CV (CORNING), with 4.5 g/L glucose, L-glutamine, sodium pyruvate) was employed. 1 tube of FBS (50 mL), 1 tube of Penicillin/Streptomycin (2.5 mL) and 1 tube of Amphotericin B (1 mL) were added to 1 bottle of Corning DMEM (500 mL). This complete medium was employed in pH experiments as test medium X.

Buffered test medium Y was prepared from test medium X with added HEPES buffer at 20 mM. 1M HEPES was used in 50 mL DMEM (X). 1 mL of 1M HEPES was added to 49 mL of DME M (X) to obtain 20 mM solution. Well mixed.

Buffered test Medium Z was prepared from test medium X with added sodium bicarbonate at 3.024 g/L. 151.2 mg of Bicarbonate was added to 50 mL of DMEM (X) mixed well.

### Preparation of Test Compositions for pH Testing

Stock Solution A: 1 tube of Stock Solution A of water-soluble compounds, as follows: The following was dissolved in sterile water for injection: 3002.0 mg L-Lysine monohydrochloride (182.65 g/mol), 280.4 mg ascorbic acid (176.13 g/mol), 640.2 mg calcium ascorbate dihydrate (426.34 g/mol), 300.0 mg niacinamide ascorbate (298.25 g/mol), 50.0 mg pyridoxine HCl (205.64 g/mol), 200.0 mg taurine (125.15 g/mol), 50.0 mg threonine (119.12) into 15 mL water. Mixed, until fully dissolved and clear solution. Sterile filtered into a sterile glass vial.

Stock Solution B: 1 tube of water-insoluble compounds. The following was dissolved in 5 mL of DMSO: 83.3 mg ascorbyl palmitate (414.54 g/mol), 499.6 mg hesperidin (610.62 g/mol) and 514.2 mg Rutin hydrate (628.5 g/mol) in 5 mL DMSO. Mixed until fully dissolved and clear solution. Sterile filter into a sterile glass vial.

Working Stocks 1X, 1Y, 1Z were made as follows. Add 1.5 mL of Solution A to 13.2 mL of each Test Medium X, Y or Z. Add 0.3 mL Solution B to each tube and mix to make Working Stock 1X, 1Y, or 1Z that is 2% DMSO in DMEM. Use immediately. If there is no loss to filtration, 300.07 mg/15.0 mL=20.01 mg/mL L-lysine monohydrochloride (110 mmol/L lysine equivalent).

Working Stocks 2X, 2Y, 2Z were made as follows. Dilute Working Stocks 1X, 1Y, 1Z with appropriate warm “Test Medium X”, “Test Medium Y”, or “Test Medium Z”, respectively, to 50 mM lysine equivalent as follows. Add 4.55 mL “Working Stock 1×” to 5.45 mL “Test Medium X” and mix to make 10 mL Working Stock 2X. (50.05 mmol/L). Add 4.55 mL “Working Stock 1Y” to 5.45 mL “Test Medium Y” and mix to make 10 mL Working Stock 2Y. (50.05 mmol/L lysine equivalent). Add 4.55 mL “Working Stock 1Z” to 5.45 mL “Test Medium Z” and mix to make 10 mL Working Stock 2Z. (50.05 mmol/L).

The appearance and pH of the solutions was measured as shown in Table 4. All experiments were done at 37° C. immediately from incubator. Small sample of Solution A was used for this measurement.

TABLE 4
pH of Test Compositions in Buffered and Unbuffered Media

| Solution | Appearance | pH | Temp, deg C. |
|---|---|---|---|
| Solution A | Clear, colorless | 4.52 | 36.5 |
| | solution | | |
| Test Medium X | Original light red | 7.35 | 36.7 |
| | color | | |
| Test Medium Y | Original light red | 7.38 | 36.2 |
| | color | | |
| Test Medium Z | Original light red | 7.63 | 36.0 |
| | color | | |
| Working Stock 1X | Pale yellow | 6.44 | 36.3 |
| Working Stock 1Y | Pale yellow | 6.62 | 36.0 |
| Working Stock 1Z | Pale yellow | 6.83 | 36.4 |
| Working Stock 2X | Red with yellow hue | 7.22 | 36.1 |
| Working Stock 2Y | Red with yellow hue | 7.23 | 36.2 |
| Working Stock 2Z | Red with yellow hue | 7.45 | 36.4 |

Results: Bicarbonate buffered medium Z appeared to buffer solutions to neutral pH somewhat better than HEPES buffered medium Y. Cell based studies were performed in bicarbonate buffered medium Z and unbuffered medium X.

### Example 3. Cell Based Antiviral Studies—Herpes Simplex Virus 1 and 2 in ARPE19 Cell Culture in Buffered and Unbuffered Media

The goal of this study was to evaluate the effects of full Test Composition in medium buffered with sodium bicarbonate and unbuffered medium. These assays were performed against HSV-1 and HSV-2 in ARPE-19 cells, and the different solutions/compounds were added at the time of infection. The effects of original Test Composition were compared to acyclovir, which is the gold standard for HSV treatment.

The efficacy of original Test Composition against HSV-1 had been tested in ARPE-19 cells (spontaneously arising retinal pigment epithelial cell line) several times with varying conditions. However, it was consistently noted that the media changed colors, and it was presumed that this was associated with the acidic nature of Test Composition and its components. To combat this, the media was tested as shown in in example 2 to determine the optimal buffering conditions. Once media conditions were selected, Test Composition 1 was tested in unbuffered medium or in medium buffered with sodium bicarbonate against HSV-1 and HSV-2.

Briefly, cells were seeded into 96-well plates and grown in DMEM medium overnight, reaching 75-80% confluence after 24 h. Next, cells were infected at a MOI of 0.05 with either HSV-1 R8411 or HSV-2-Luc, both which expresses firefly luciferase, in either standard DMEM medium (10% FBS) or standard DMEM medium (10% FBS) buffered with 3.024 g/L sodium bicarbonate. Treatments were prepared in the different medium conditions and added at the time of infection. All assays were imaged at 24 h post-infection and/or treatment. The compounds tested for this experiment included: Test Composition in unbuffered medium, Test Composition in buffered medium, acyclovir in unbuffered medium and acyclovir in buffered medium. All graphs, EC50, CC50, and CC50/EC50 calculations were performed using GraphPad software based on Lysine equivalent concentrations.

HSV-1 or HSV-2 infected cells were measured by bioluminescence imagining in the dose-response assay. Each plate contained a set of untreated wells in each media condition (unbuffered or buffered) to be used as baseline. Test composition concentrations ranged from 3.2 μM to 50 mM in a 1:5 dilution series. Acyclovir concentrations ranged from 0.00256 to 40 μM in a 1:5 dilution series.

For the cell effect assay in absence of virus, ARPE-19 cells were seeded into 96-well plates and grown for 24 h as described above. Cells were treated with Test Composition in either unbuffered or buffered medium ranging from 1 μM to 100 mM (1:10 dilution) based on Lysine concentrations. Acyclovir was tested in a range from 0.001 to 100 μM (1:10 dilution). After 24 h of treatment, cells were stained with Neutral Red dye and absorbance was measured at 540 nM.

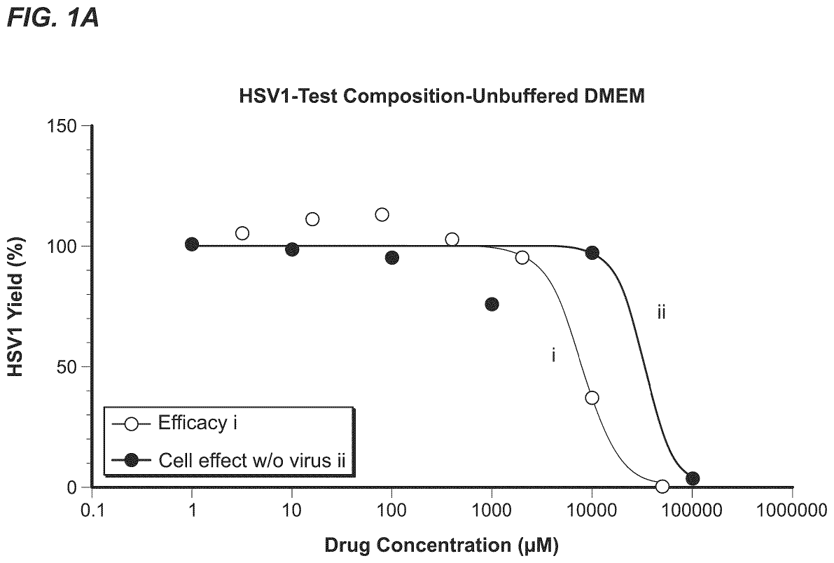

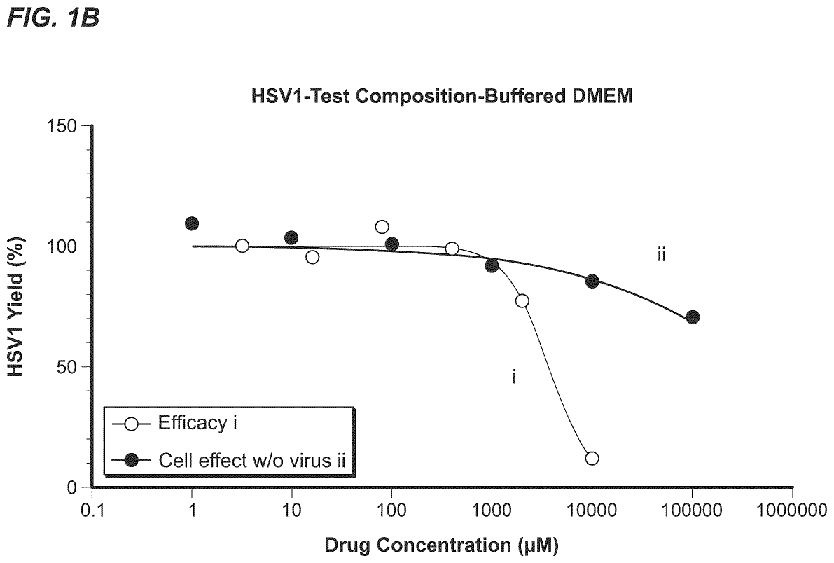

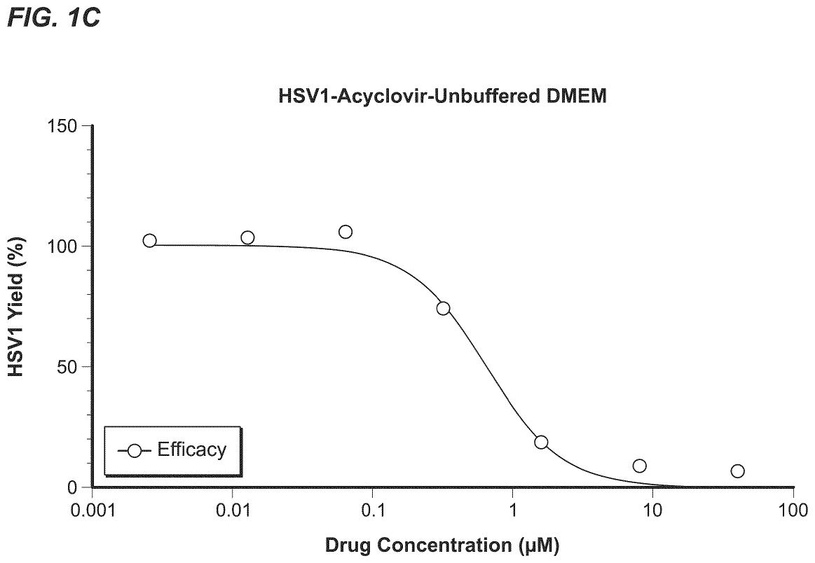

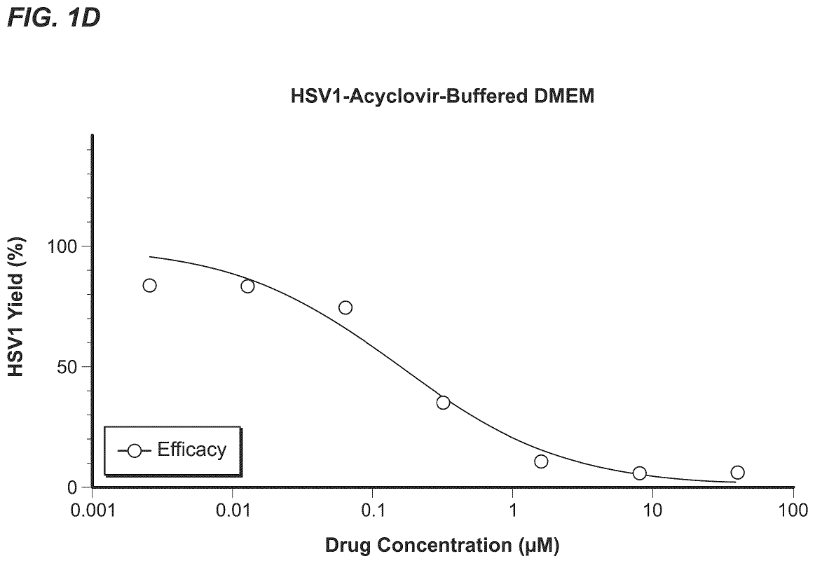

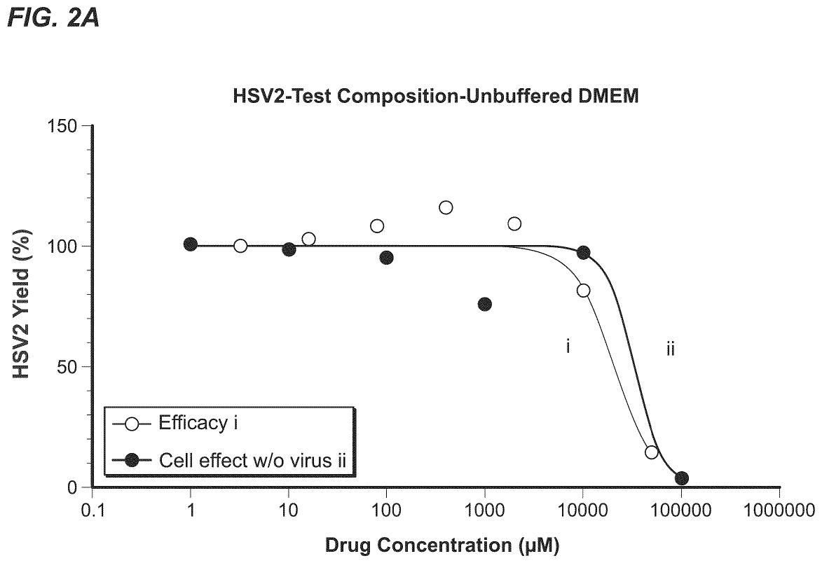

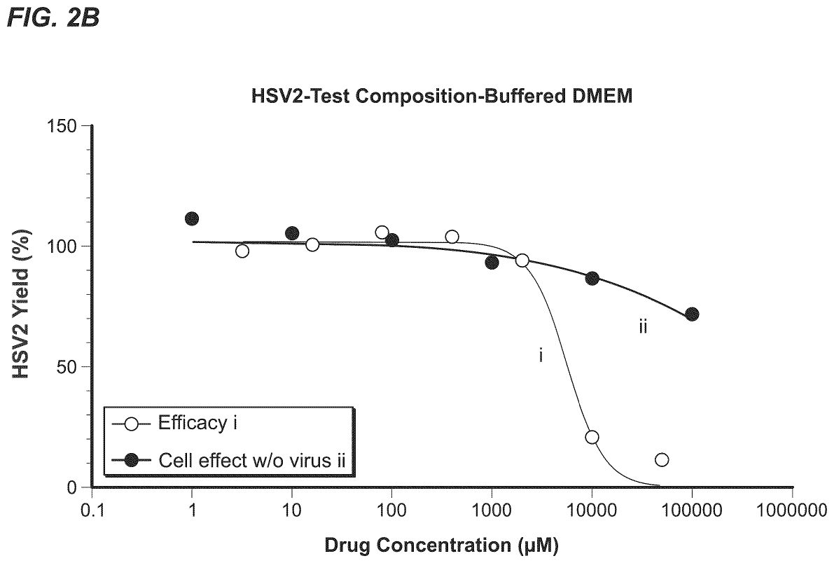

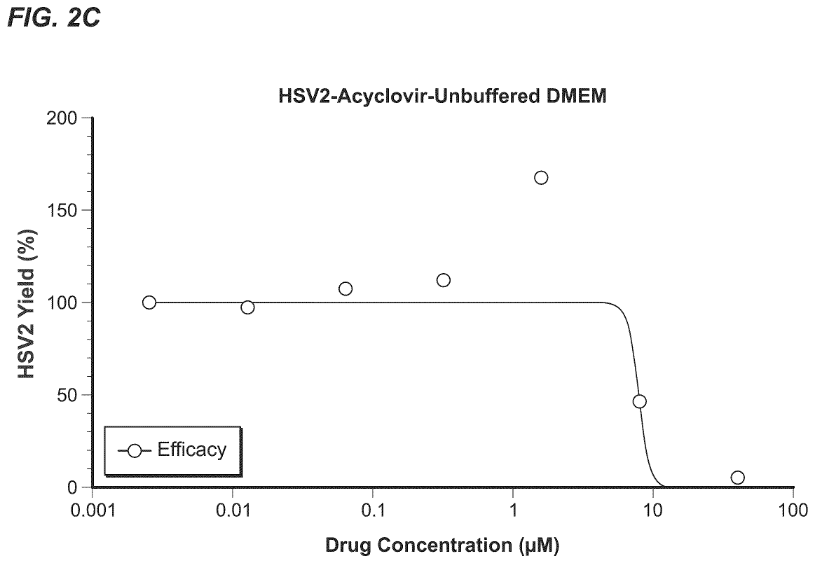

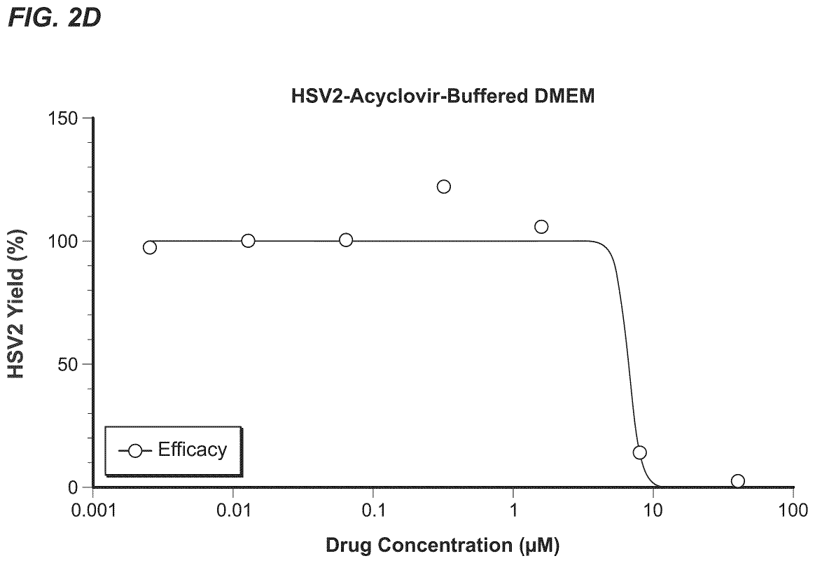

Results: To evaluate the efficacy of Test Composition in buffered versus unbuffered medium against HSV-1 and HSV-2, infected and treated plates were imaged at 24 h post-infection with the IVIS® bioluminescence imagining system (Revvity). As expected, the control drug acyclovir was effective against both viruses at 24 h post-infection. Test Composition was effective against HSV-1 and HSV-2 in both unbuffered and buffered medium, but to differing extents. FIG. 1A-D (HSV-1) and FIG. 2A-D (HSV-2) show the curves used to calculate the 50% efficacy concentration, EC50, green curve, (i); 50% cytotoxic concentration or cell effect without virus (e.g., cell density(%)), CC50, dark red curve, (ii) and selectivity index (SI, CC50/EC50=SI) for each media condition. These values are presented in summary table 5 (HSV-1) and table 6 (HSV-2) below. An SI value near or above 10 is considered to be a good hit and active in vitro. In the cell effect without virus curves, excess precipitation in the wells was noted at the highest concentrations, however this precipitation was much higher with the unbuffered medium than the buffered medium.

TABLE 5
Antiviral Efficacy vs. HSV-1 in ARPE-19 cells

| | Unbuffered DMEM (in μM) | | | Buffered DMEM (in μM) | | |
|---|---|---|---|---|---|---|
| | EC₅₀ | CC₅₀ | SI | EC₅₀ | CC₅₀ | SI |
| Test | 7946 | 32,965 | 4 | 3672 | >100,000 | 27 |
| Composition | | | | | | |
| Acyclovir | 0.6448 | — | — | 0.1556 | — | — |

TABLE 6
Antiviral Efficacy vs. HSV-2 in ARPE-19 cells

| | Unbuffered DMEM | | | | | |
|---|---|---|---|---|---|---|
| | (in μM) | | | Buffered DMEM (in μM) | | |
| | EC₅₀ | CC₅₀ | SI | EC₅₀ | CC₅₀ | SI |
| Test | 20886 | 32,965 | 1.6 | 5640 | >100,000 | 17.7 |
| Composition | | | | | | |
| Acyclovir | 7.894 | — | — | 6.662 | — | — |

Test Composition was effective against HSV-1 and HSV-2 in both unbuffered and buffered medium. Using buffered medium, Test Composition exhibited acceptable selectivity (SI) index values of 27 and 17.7 in HSV-1 or HSV-2 infected cells, respectively.

### Example 4. Cell Based Antiviral Studies—Human Cytomegalovirus and Varicella Zoster Virus in Buffered and Unbuffered Medium

The goal of this study was to evaluate the effects of original Test Composition against two human herpesviruses, varicella zoster virus (VZV), which causes chicken pox (varicella) and shingles (herpes zoster), and human cytomegalovirus (HCMV), which is the leading cause of viral congenital birth defects. The difference in effects of medium buffered with sodium bicarbonate versus unbuffered medium was also of interest.

The assays performed against VZV were completed in ARPE-19 cells (spontaneously arising retinal pigment epithelial cells line), while the assays performed against HCMV were completed in human foreskin fibroblast cell line (HFFs). For all assays, the different solutions/compounds were added at the time of infection. The effects of original and soluble Test Compositions were compared to cidofovir or ganciclovir, which are standard for VZV and HCMV treatment, respectively.

Original Test Composition was found to be effective against the human herpesviruses herpes simplex virus 1 (HSV-1) and 2 (HSV-2) in ARPE-19 cells (spontaneously arising retinal pigment epithelial cell line). Thus, it was sought to investigate the effects of Test Compositions against VZV and HCMV in ARPE-19 cells and human foreskin fibroblasts (HFFs), respectively. Some differing effects were observed using buffered versus unbuffered medium in previous assays, so cell based antiviral tests against these viruses were performed in both medium conditions.

VZV- or HCMV-infected cells were measured by bioluminescence imagining in the efficacy or dose-response assay. Assays were performed as previously described, with a few modifications. Briefly, cells were seeded into 96-well plates and grown in DMEM medium for 3 days, reaching confluence prior to infection. Next, cells were infected at a MOI of approximately 0.05 with either VZV-ORF57-Luc or HCMV-eGFP-fLuc, both which expresses firefly luciferase, in either standard DMEM (10% FBS) or DMEM (10% FBS) buffered with 3.024 g/L sodium bicarbonate.

Antiviral Test Compositions were prepared in the two media, added at the time of infection, and remained for the duration of the assay. VZV cultures were scanned at 72 h post-infection, and HCMV cultures were scanned at 7 days post-infection. Each plate contained untreated wells in each media condition (unbuffered or buffered) as a baseline. Test Composition in unbuffered medium, Test Composition in buffered medium, cidofovir positive control (CDV; for VZV) or ganciclovir positive control (GCV; for HCMV) in unbuffered medium, and CDV (for VZV) or GCV (for HCMV) in buffered medium. All graphs, EC50, and CC50 calculations were performed using GraphPad. Test Composition concentrations ranged from 3.2 μM to 50 mM in a 1:5 dilution series based on Lysine concentrations. Cidofovir concentrations ranged from 0.00128 to 20 μM in a 1:5 dilution series. Ganciclovir concentrations ranged from 0.0064 to 100 μM in a 1:5 dilution series.

For the cell effect in absence of virus assay, ARPE-19 cells or HFFs were seeded into 96-well plates and grown for 72 h as described above. Cells were treated with Test Composition in either unbuffered or buffered medium ranging from 1 μM to 100 mM (1:10 dilution). Cidofovir was tested in a range from 0.001 to 100 μM (1:10 dilution). Ganciclovir was tested in a range from 0.002 to 200 μM (1:10 dilution). ARPE-19 cells were treated with Test Composition or Cidofovir for 72 h, then cells were stained with Neutral Red dye and absorbance was measured at 540 nM. HFFs were treated with original Test Composition or Ganciclovir for 7 d, then cells were stained with Neutral Red dye and absorbance was measured at 540 nM.

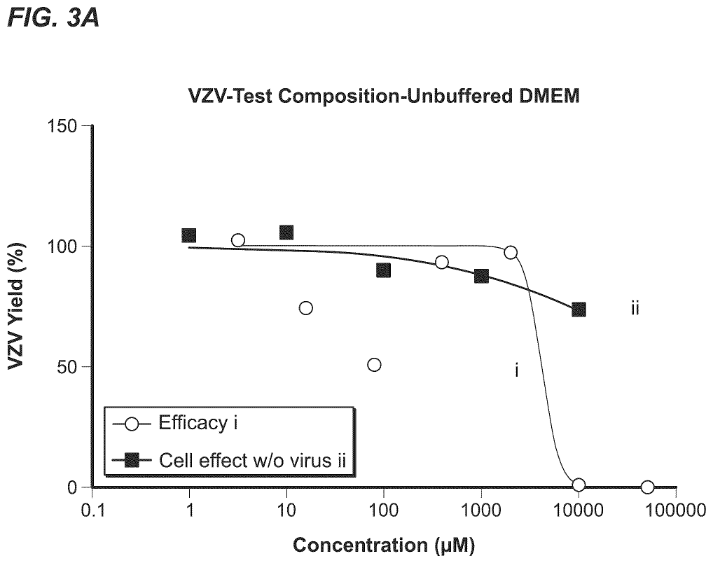

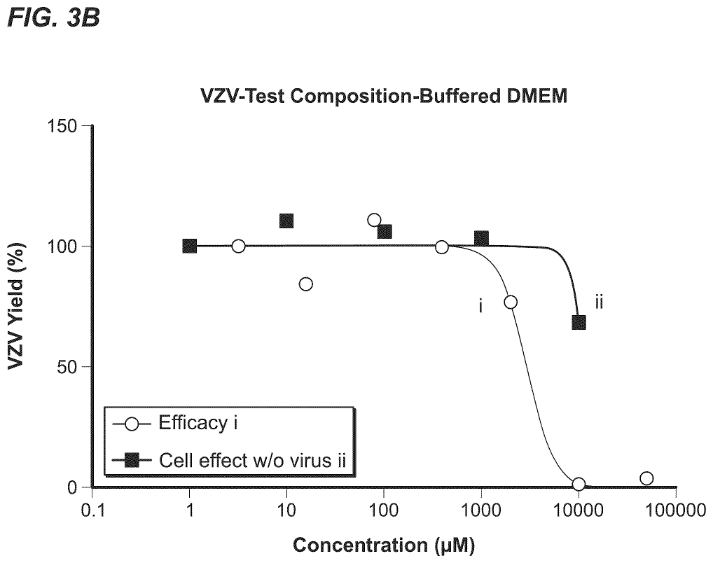

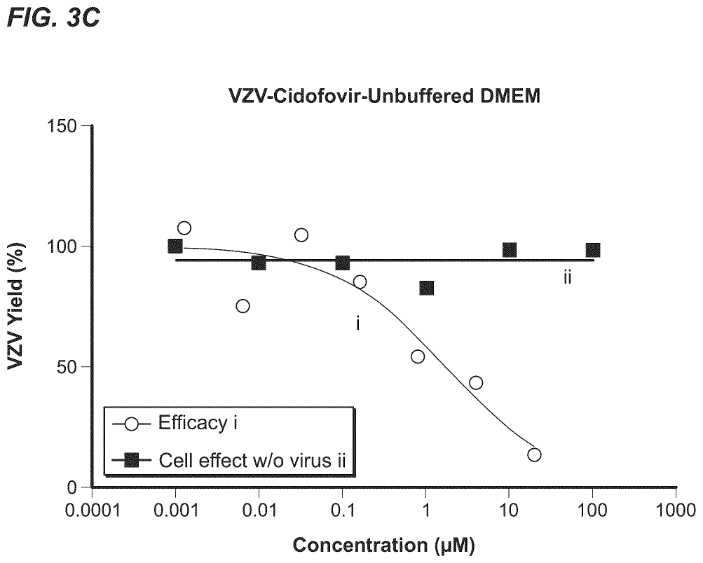

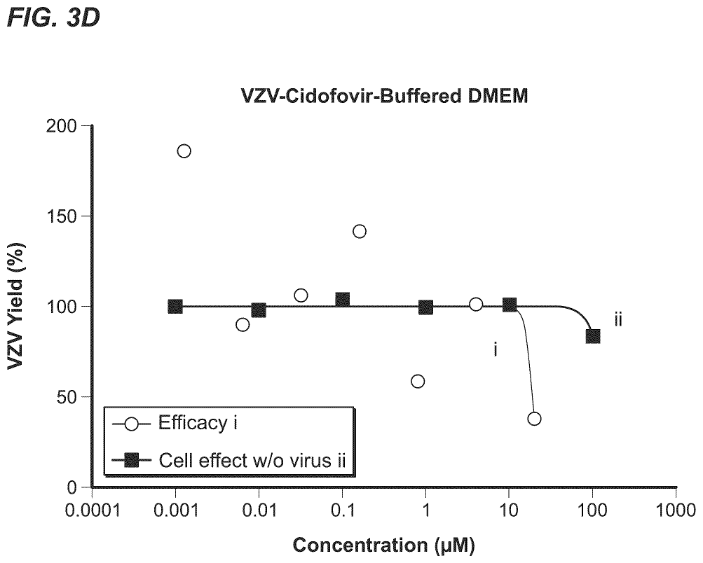

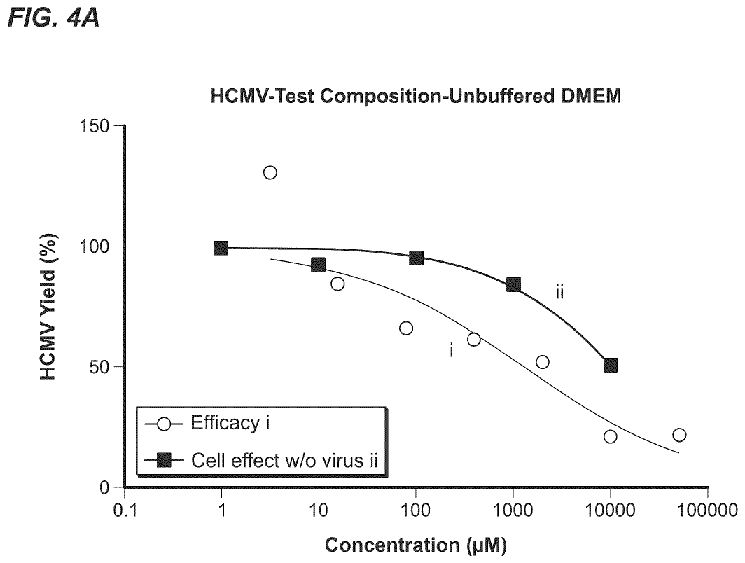

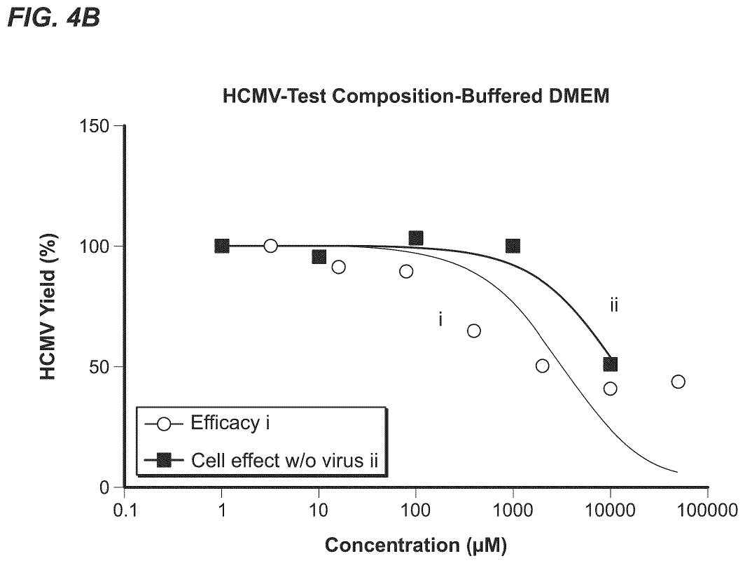

Results: The positive control drug cidofovir was effective against VZV, as expected. Unfortunately, the positive control drug ganciclovir was not effective against HCMV, likely due to degradation of the stock solution, data not shown. Test Composition was effective against both VZV and HCMV in unbuffered and buffered medium as shown in FIG. 3A-D and FIG. 4A-B, but to differing extents. The 50% efficacy concentration (EC50, green curve), 50% cytotoxic concentration (CC50, dark red curve) and selective index (SI, CC50/EC50=SI) for each treatment and medium condition are shown in Table 7 and Table 8. An SI value near or above 10 is considered a good hit. In the cell effect in absence of virus assay, excess precipitation in the wells was noted at the highest concentrations, and this precipitation was greater in the unbuffered medium.

TABLE 7
Results for varicella zoster virus (VZV)

| | Unbuffered DMEM (in μM) | | | Buffered DMEM (in μM) | | |
|---|---|---|---|---|---|---|
| | EC₅₀ | CC₅₀ | SI | EC₅₀ | CC₅₀ | SI |
| Test | 4157 | >10,000 | 2.4 | 2956 | >10,000 | 3.4 |
| Composition | | | | | | |
| Cidofovir | 1.717 | >100 | 58.2 | 19 | >100 | 5.3 |

TABLE 8
Results for human cytomegalovirus (HCMV)

| | Unbuffered DMEM (in μM) | | | Buffered DMEM (in μM) | | |
|---|---|---|---|---|---|---|
| | EC₅₀ | CC₅₀ | SI | EC₅₀ | CC₅₀ | SI |
| Test | 1358 | >10,000 | 7.4 | 3212 | >10,000 | 3.1 |
| Composition | | | | | | |
| Ganciclovir | n.d. | >200 | — | n.d. | >200 | — |

Discussion. The Test Composition in buffered and unbuffered medium was effective against VZV and HCMV, although to differing extents. For VZV, the EC50 was lower in buffered medium, resulting in higher SI values. Unfortunately, the CC50 value could not be calculated in either medium, as Test Composition precipitated at the highest concentration (100 mM) and it interfered with the assay. Interestingly, the buffered medium affected VZV growth. VZV grew better in the unbuffered medium compared to the buffered medium. To account for this, Test Composition effects were compared directly to the untreated conditions for each medium. Unexpectedly, the EC50 for cidofovir increased 10-fold in buffered medium. This is likely due to lower VZV growth in buffered medium. For HCMV, buffering the medium made no difference in Test Composition efficacy. However, the EC50 values were slightly higher for HCMV treated with Test Composition in buffered medium.

### Example 5. Cell Based Antiviral Studies—Herpes Simplex Virus 1 and 2 in ARPE19 Cell Culture in Buffered and Unbuffered Media

The goal of this study was to evaluate the effects of the Test Composition and its different components/formulations against two human herpesviruses, herpes simplex virus 1 and 2 (HSV1/2). A sodium bicarbonate buffered medium with 10% FBS, Pen/Strep and AmpB was employed. The purpose of this example was a better understanding of the specific components of the Test Composition that lead to its antiviral effects. All assays were performed in ARPE-19 cells with the buffered medium, and the different solutions were added at the time of infection. The different combinations of Test Composition components were all compared to acyclovir, the standard for HSV treatment.

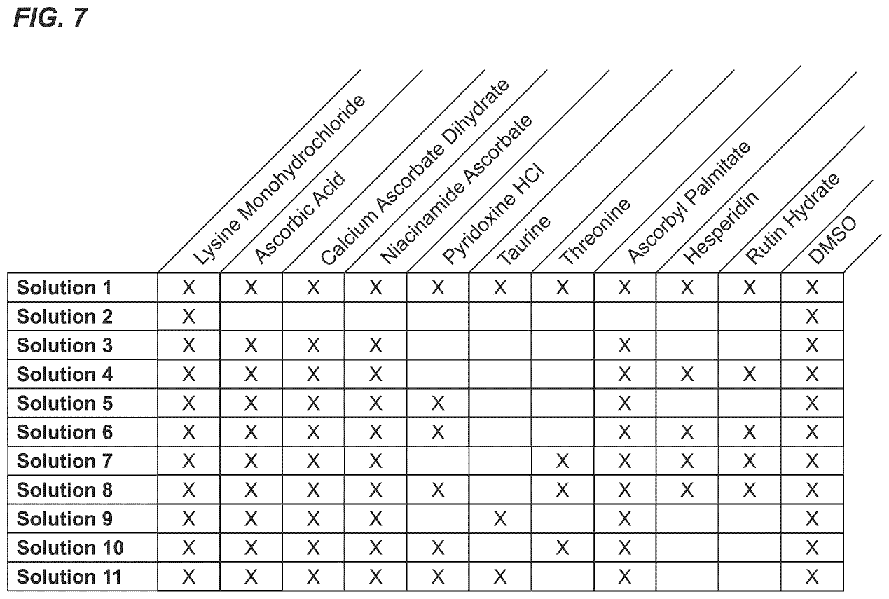

The Test Composition (Solution 1) is effective against human herpesviruses herpes simplex virus 1 (HSV1) and 2 (HSV2) in ARPE-19 cells (spontaneously arising retinal pigment epithelial cell line). This study sought to investigate the effects of the different components of the Test Composition and various component combinations in buffered medium. For the efficacy studies, HSV1/2 were measured by bioluminescence imaging (dose response assay, yields EC50 value). Test Solutions are shown in Table 9 and are graphically illustrated in FIG. 7.

TABLE 9
Test Composition Components

| Test | Stock | |
|---|---|---|
| Solution | Factors | Components |
| 1 | A ± B | A: L-Lysine monohydrochloride, ascorbic acid, |
| | | calcium ascorbate dihydrate, niacinamide ascorbate, |
| | | pyridoxine HCl, taurine, threonine |
| | | B: ascorbyl palmitate, hesperidin, rutin hydrate, |
| | | DMSO |
| 2 | C ± D | C: L-lysine monohydrochloride |
| | | D: DMSO |
| 3 | E ± F | E: L-Lysine monohydrochloride, ascorbic acid, |
| | | calcium ascorbate dihydrate, niacinamide ascorbate |
| | | F: ascorbyl palmitate, DMSO |
| 4 | E ± B | E: L-Lysine monohydrochloride, ascorbic acid, |
| | | calcium ascorbate dihydrate, niacinamide ascorbate |
| | | B: ascorbyl palmitate, hesperidin, rutin hydrate, |
| | | DMSO |
| 5 | G ± F | G: L-Lysine monohydrochloride, ascorbic acid, |
| | | calcium ascorbate dihydrate, niacinamide ascorbate, |
| | | pyridoxine HCl |
| | | F: ascorbyl palmitate, DMSO |
| 6 | G ± B | G: L-Lysine monohydrochloride, ascorbic acid, |
| | | calcium ascorbate dihydrate, niacinamide ascorbate, |
| | | pyridoxine HCl |
| | | B: ascorbyl palmitate, hesperidin, rutin hydrate, |
| | | DMSO |
| 7 | I ± B | I: L-Lysine monohydrochloride, ascorbic acid, calcium |
| | | ascorbate dihydrate, niacinamide ascorbate, threonine |
| | | B: ascorbyl palmitate, hesperidin, rutin hydrate, |
| | | DMSO |
| 8 | J ± B | J: L-Lysine monohydrochloride, ascorbic acid, |
| | | calcium ascorbate dihydrate, niacinamide ascorbate, |
| | | pyridoxine HCl, threonine |
| | | B: ascorbyl palmitate, hesperidin, rutin hydrate, |
| | | DMSO |
| 9 | H ± F | H: L-Lysine monohydrochloride, ascorbic acid, |
| | | calcium ascorbate dihydrate, niacinamide ascorbate, |
| | | taurine |
| | | F: ascorbyl palmitate, DMSO |
| 10 | J ± F | J: L-Lysine monohydrochloride, ascorbic acid, |
| | | calcium ascorbate dihydrate, niacinamide ascorbate, |
| | | pyridoxine HCl, threonine |
| | | F: ascorbyl palmitate, DMSO |
| 11 | K ± F | K: L-Lysine monohydrochloride, ascorbic acid, |
| | | calcium ascorbate dihydrate, niacinamide ascorbate, |
| | | pyridoxine HCl, taurine |
| | | F: ascorbyl palmitate, DMSO |

Assays were performed as previously described. Briefly, cells were seeded into 96-well plates and grown in DMEM medium for 24 h. Next, cells were infected at a MOI of approximately 0.05 with either HSV-1 R8411 or HSV-2 MS-Luciferase, both which expresses firefly luciferase, in DMEM (10% FBS) buffered with 3.024 g/L sodium bicarbonate. Antiviral compounds were prepared in the buffered media, added at the time of infection, and remained for the duration of the assay. Both HSV1 and HSV2 cultures were scanned at 24 h post-infection. Each plate contained untreated wells as a baseline for comparison. The solutions evaluated included original Test Composition and various sets of its components, as well as acyclovir (ACV; control). The table below includes a breakdown of all the different component formulations. Graphs were produced, EC50, and CC50 calculations were performed using GraphPad. Concentrations for Test Composition and its components ranged from 3.2 μM to 50 mM in a 1:5 dilution series. Acyclovir concentrations ranged from 0.00256 to 40 μM in a 1:5 dilution series. For the cell effect in absence of virus assay, ARPE-19 cells were seeded into 96-well plates and grown for 24 h as described above. Cells were treated with Test Composition or its components in buffered medium ranging from 1 μM to 100 mM (1:10 dilution). Acyclovir was tested in a range from 0.001 to 100 μM (1:10 dilution). ARPE-19 cells were treated with Vymune, its components, or Cidofovir for 24 h, then cells were stained with Neutral Red dye and absorbance was measured at 540 nm.

### Results:

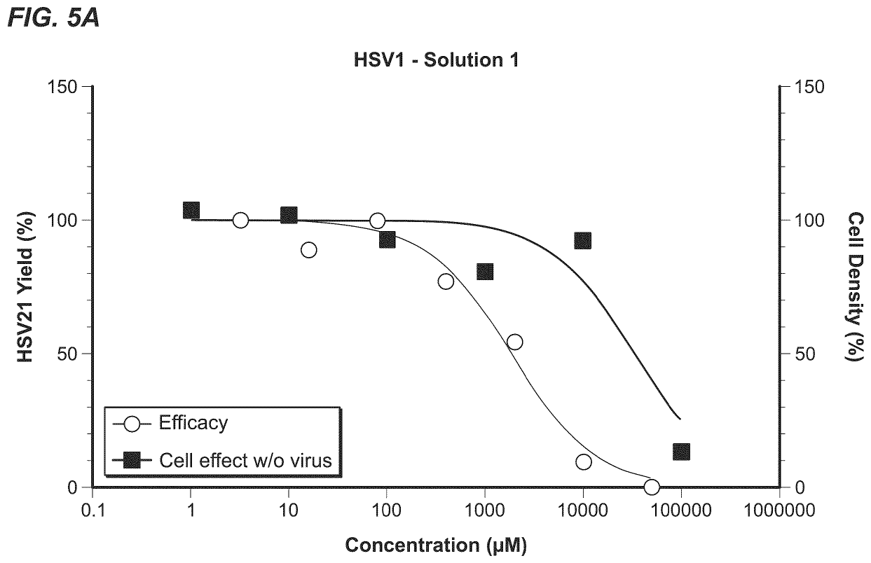

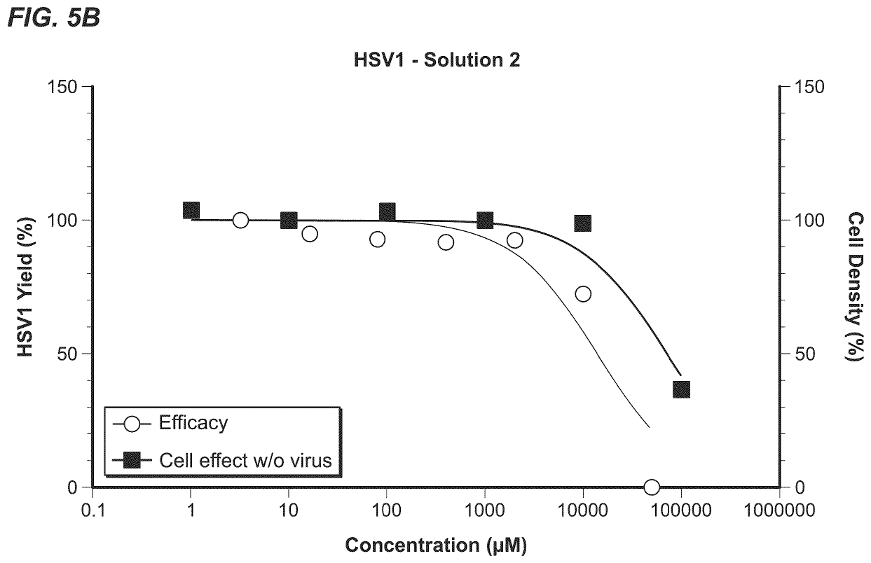

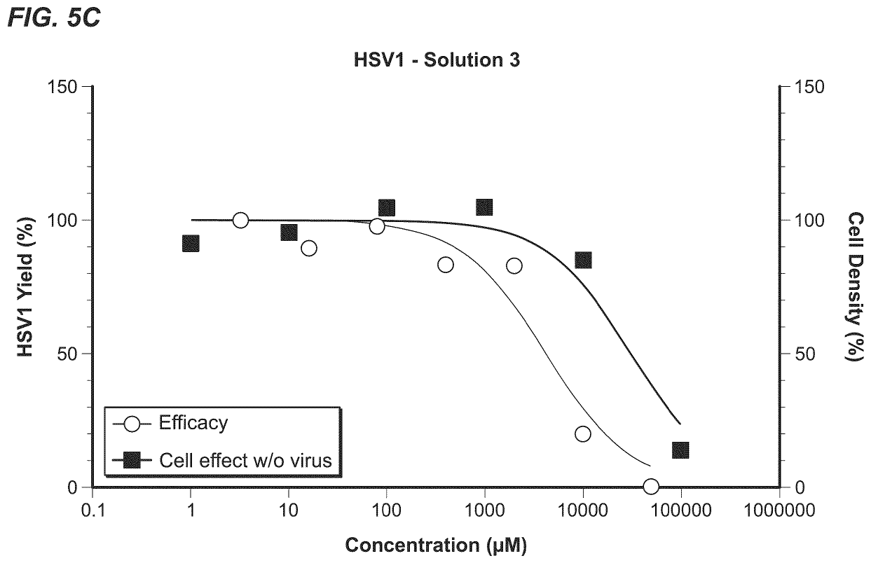

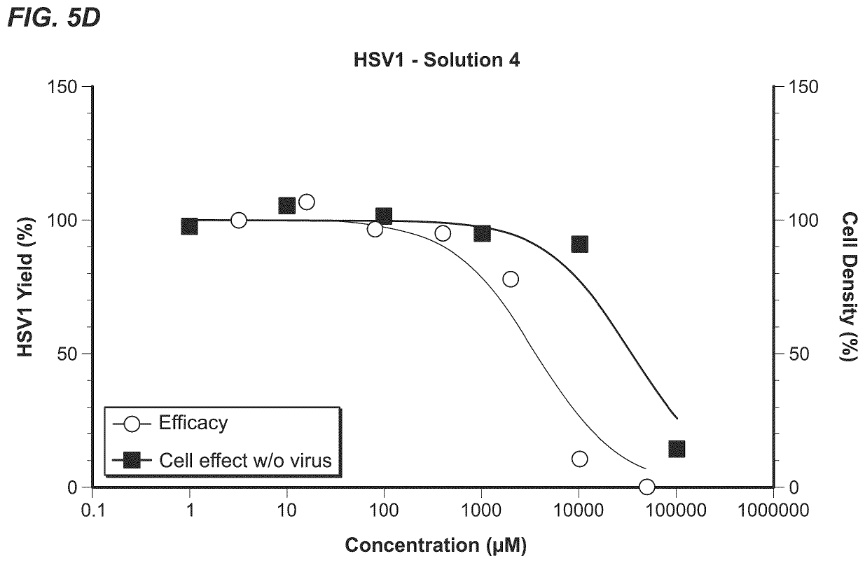

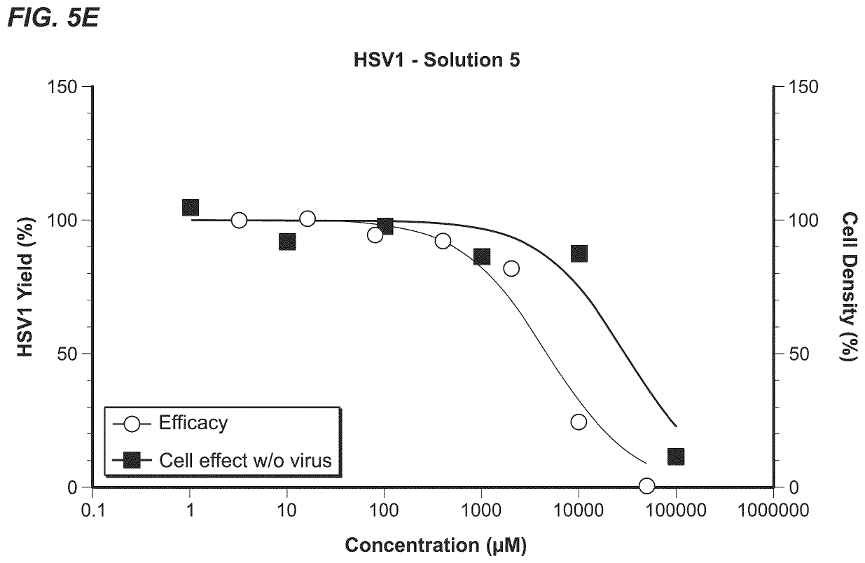

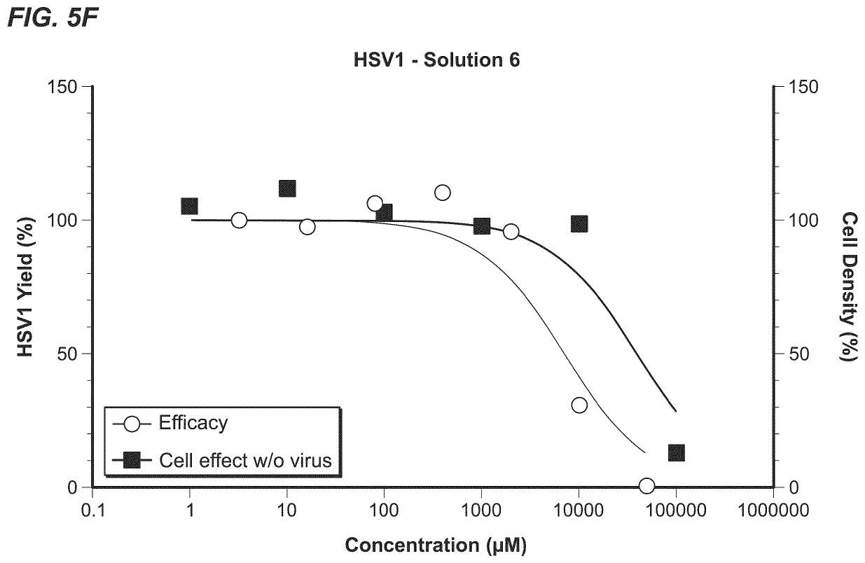

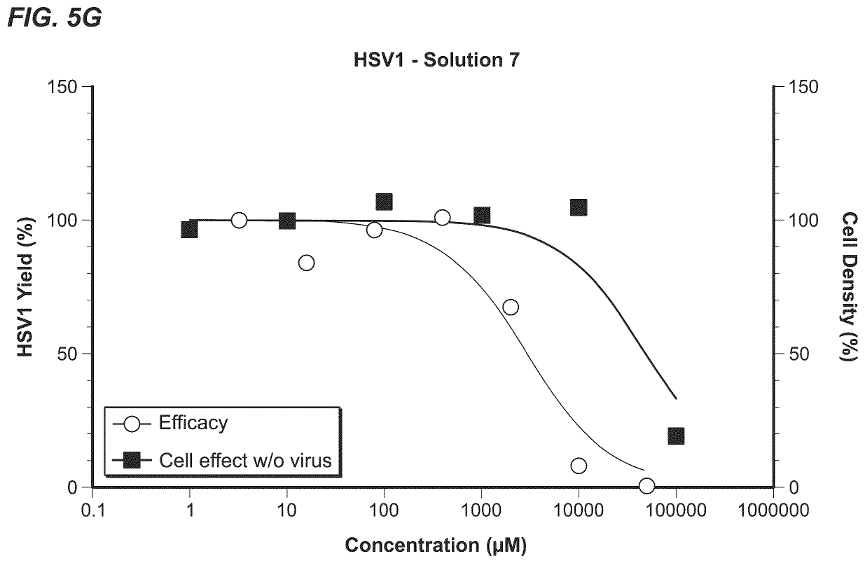

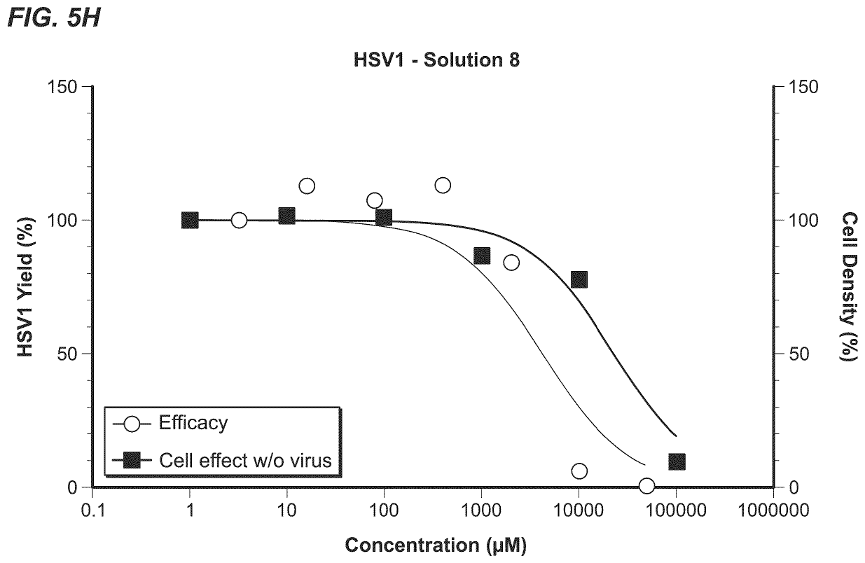

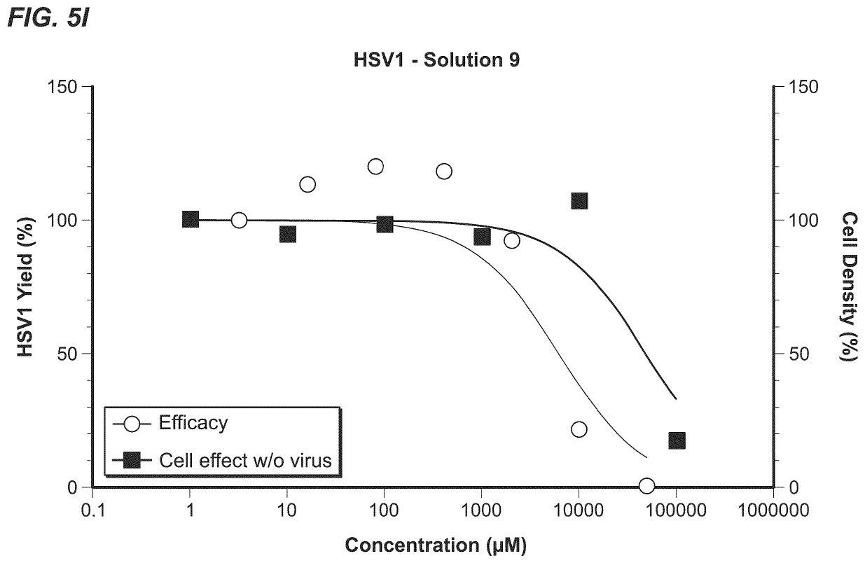

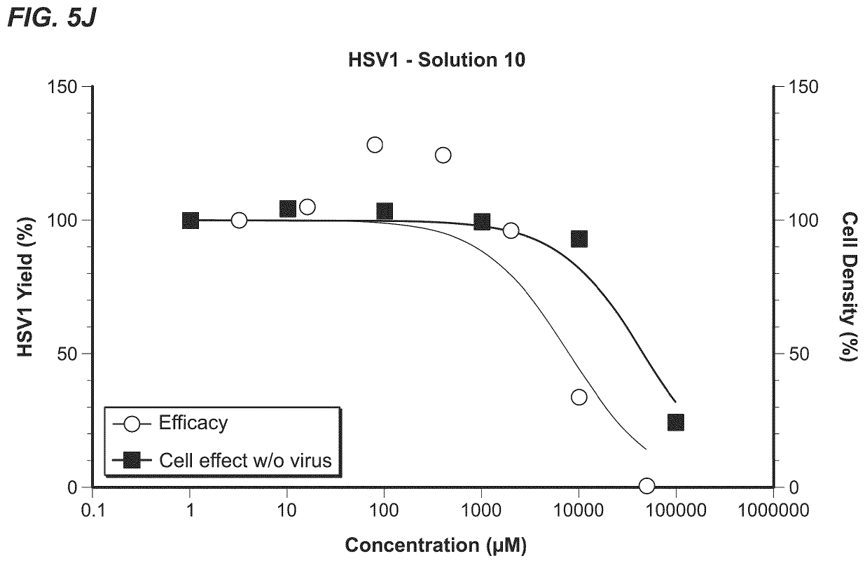

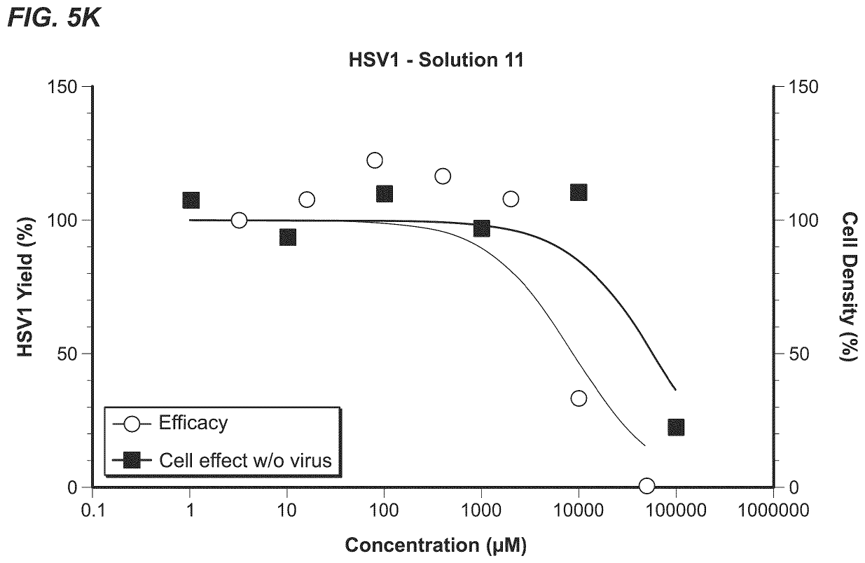

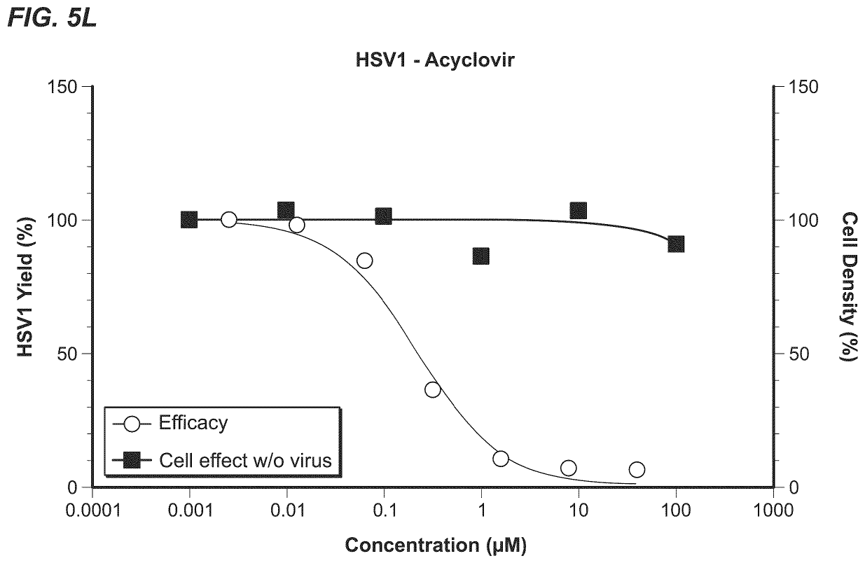

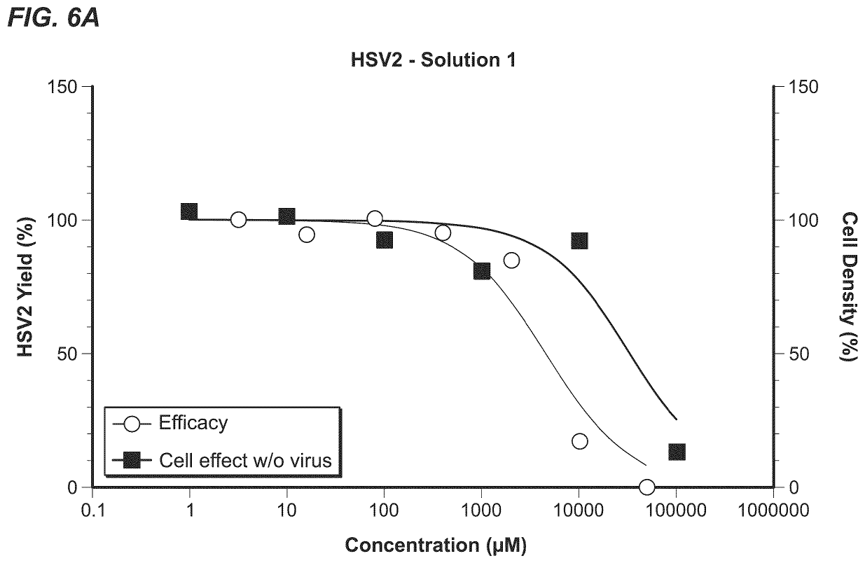

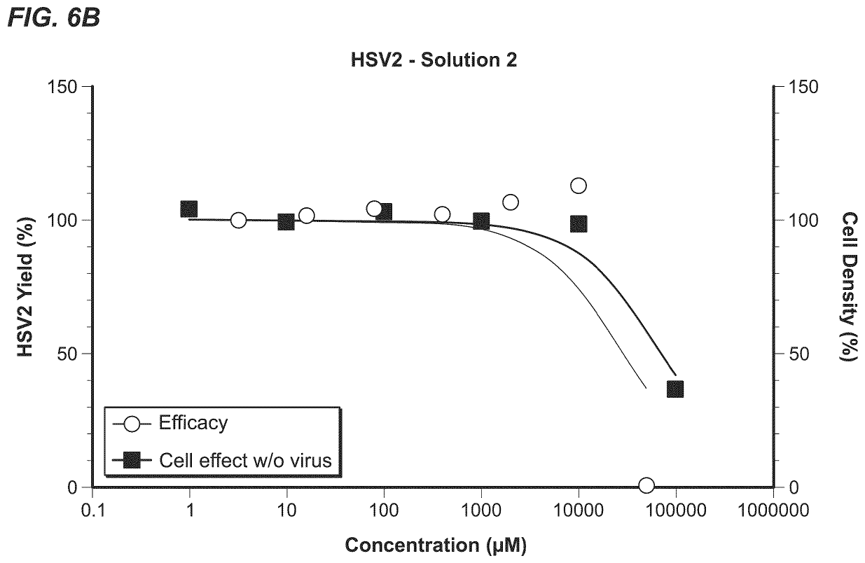

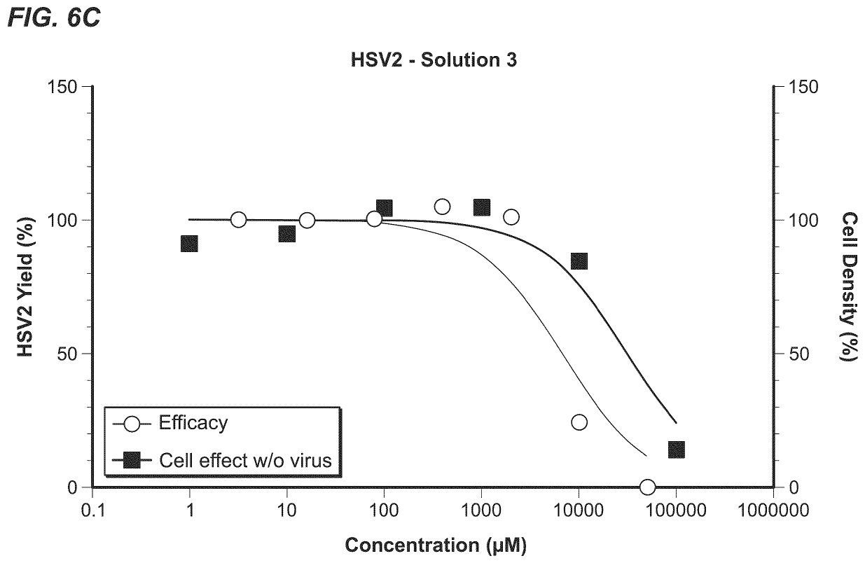

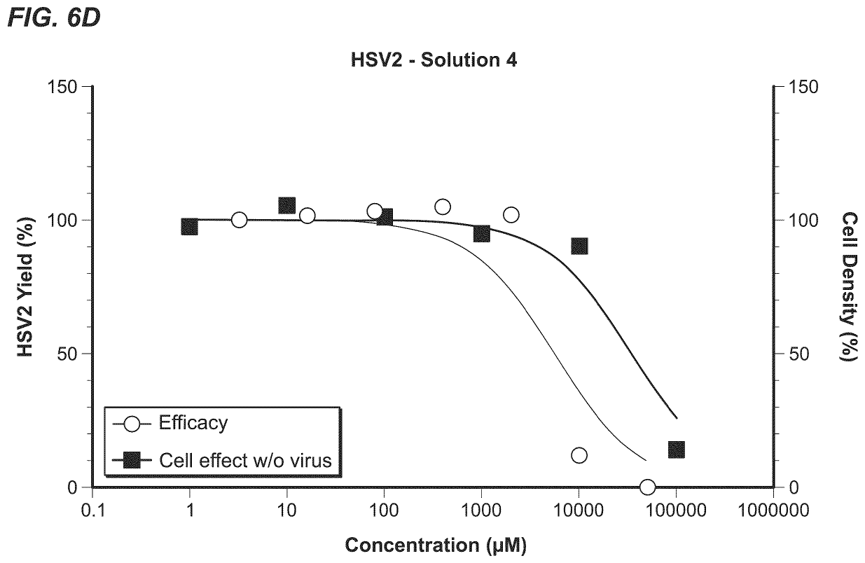

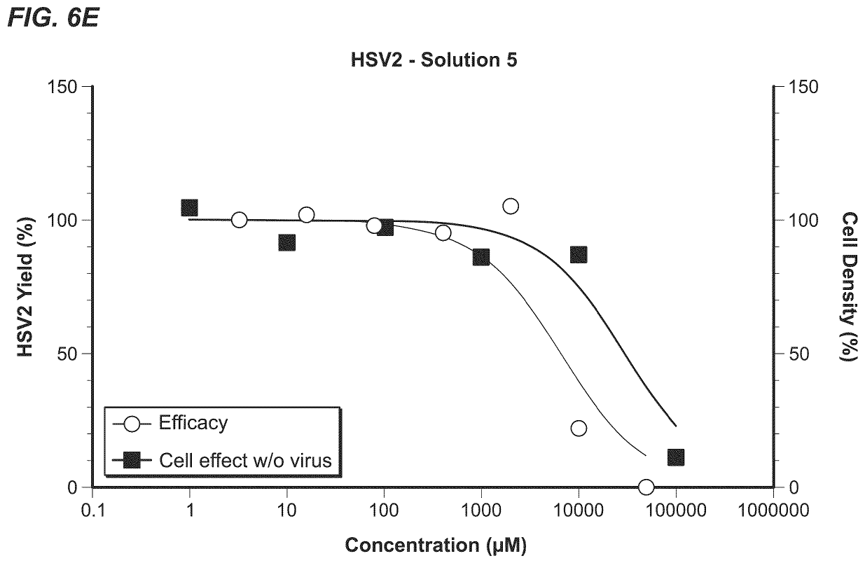

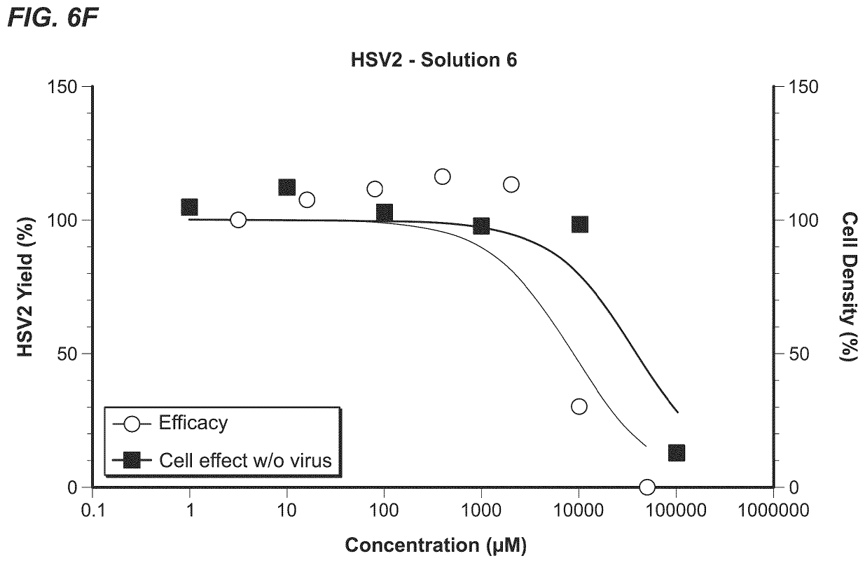

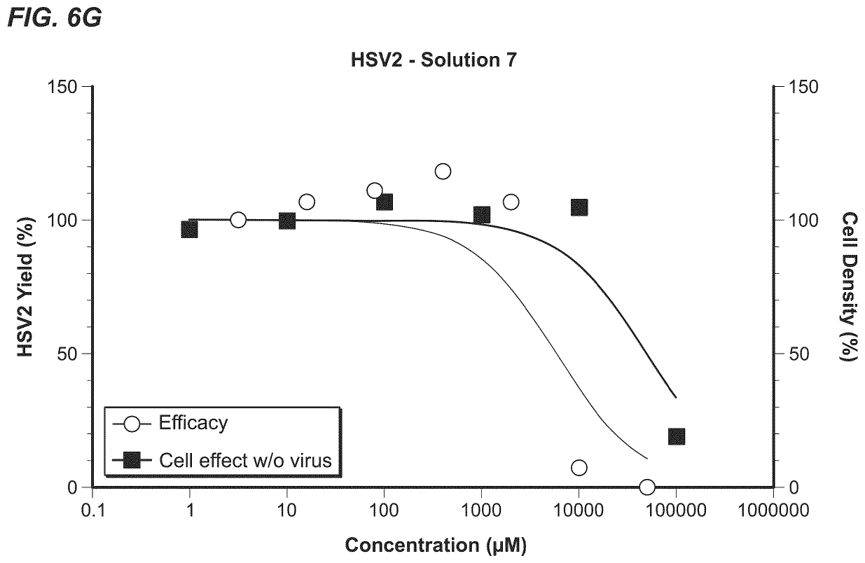

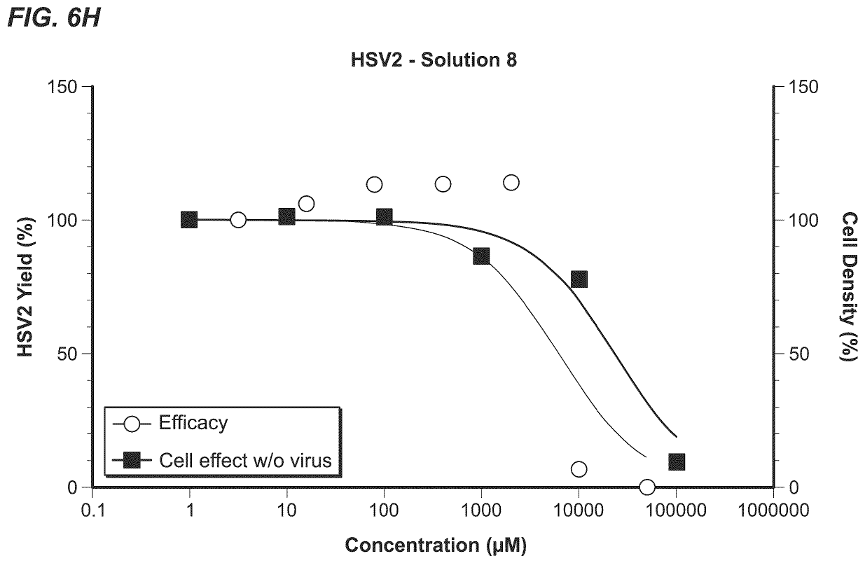

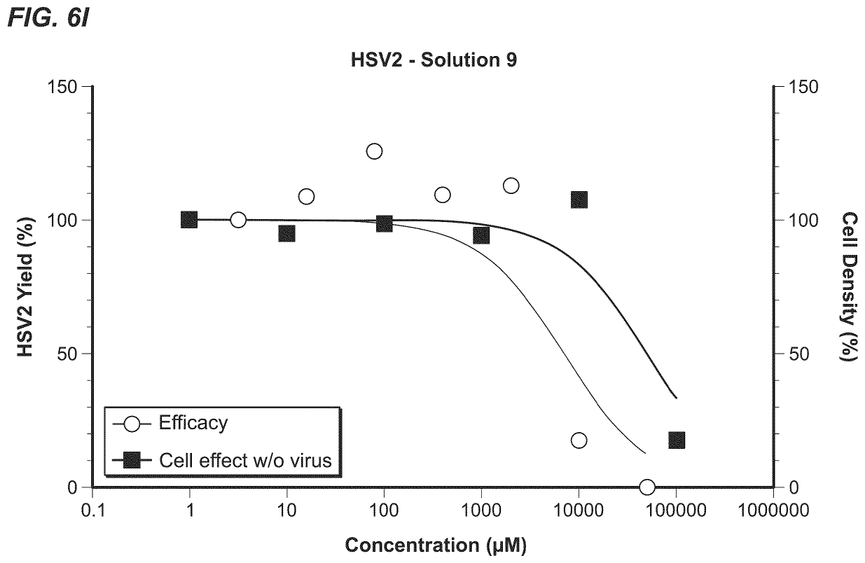

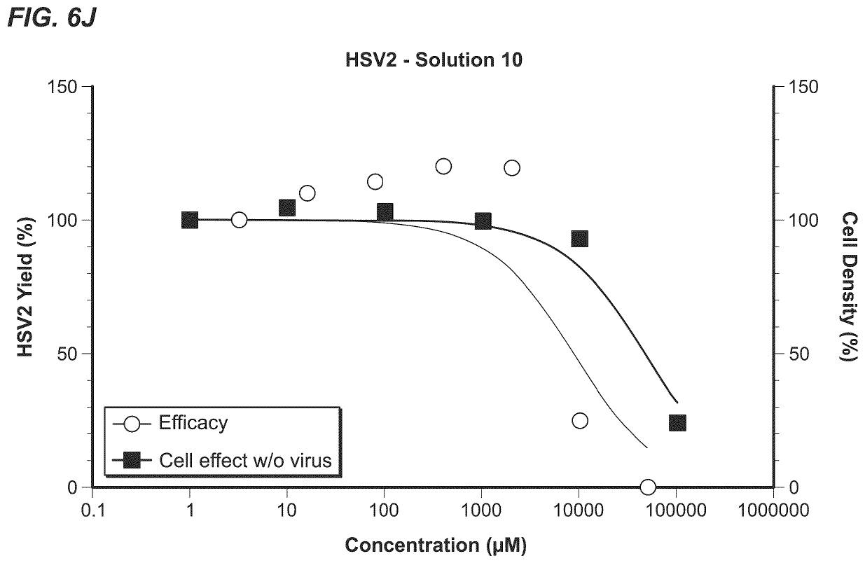

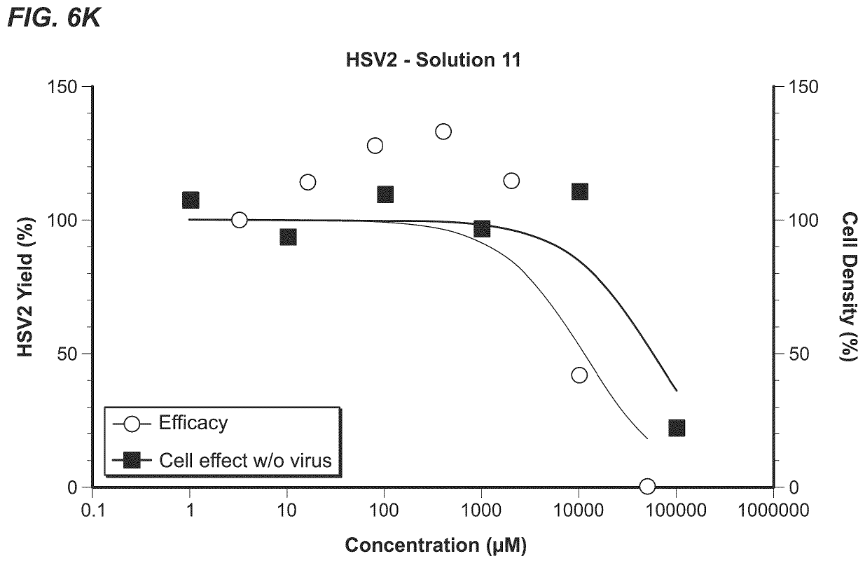

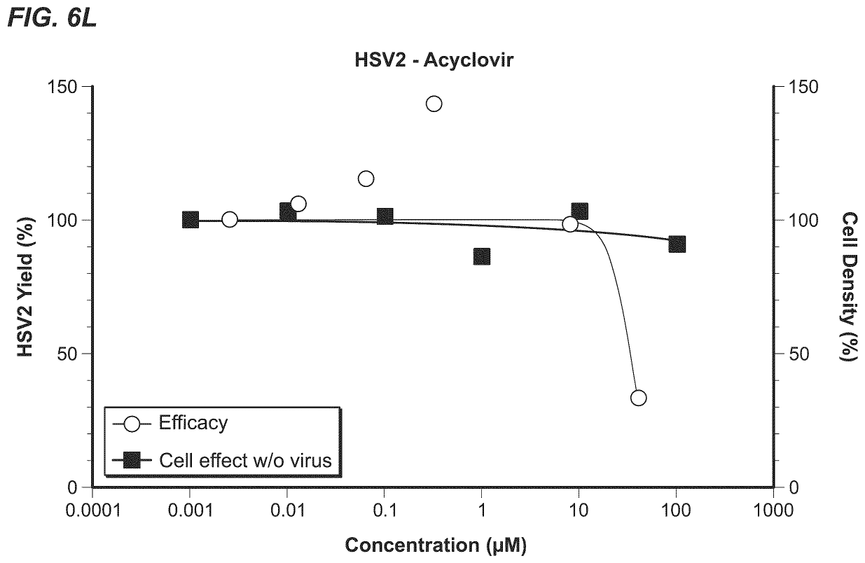

To evaluate the efficacy of the full Test Composition (Solution 1) and its different components against HSV1 and HSV2, the infected and treated plates were imaged at 24 h post-infection with the IVIS system. As expected, the control drug acyclovir was effective against both viruses at 24 h post-infection. Furthermore, the full Test Composition and its different component sets were effective against HSV1 and HSV2, but to differing extents depending on the specific components included in the solution. The curves used to calculate the 50% efficacy concentration (EC50, filled circle curve), 50% cytotoxic concentration (CC50, filled square curve) and selective index (SI, CC50/EC50=SI) for each condition are shown in FIG. 5A-L (HSV1) and FIG. 6A-L (HSV2). These values are also presented in summary Table 10. An SI value near or above 10 is considered a good hit. In the cell effect in absence of virus curves, excess precipitation in the wells was noted at the highest concentrations for some solutions, but it was mostly washed away and did not affect the assay results.

TABLE 10
Efficacy and Cell Effect in absence of virus of Test Solutions

| | HSV1 | | | HSV2 | | |
|---|---|---|---|---|---|---|
| Solution | EC50 | CC50 | SI | EC50 | CC50 | SI |
| 1 | 1855 | 33483 | 18.05 | 4520 | 33483 | 7.41 |
| 2 | 13993 | 71675 | 5.12 | 28927 | 71675 | 2.48 |
| 3 | 4211 | 31518 | 7.48 | 6645 | 31518 | 4.74 |
| 4 | 3602 | 34535 | 9.59 | 5544 | 34535 | 6.23 |
| 5 | 4721 | 29405 | 6.23 | 6472 | 29405 | 4.54 |
| 6 | 7032 | 38921 | 5.53 | 8673 | 38921 | 4.49 |
| 7 | 2978 | 49153 | 16.51 | 5810 | 49153 | 8.46 |
| 8 | 4197 | 22891 | 5.45 | 6153 | 22891 | 3.72 |
| 9 | 6158 | 48701 | 7.91 | 7018 | 48701 | 6.94 |
| 10 | 7982 | 46270 | 5.80 | 8590 | 46270 | 5.39 |
| 11 | 8715 | 56790 | 6.52 | 11139 | 56790 | 5.10 |
| Acyclovir | 0.225 | 100 | 444.44 | 33.06 | 100 | 3.02 |

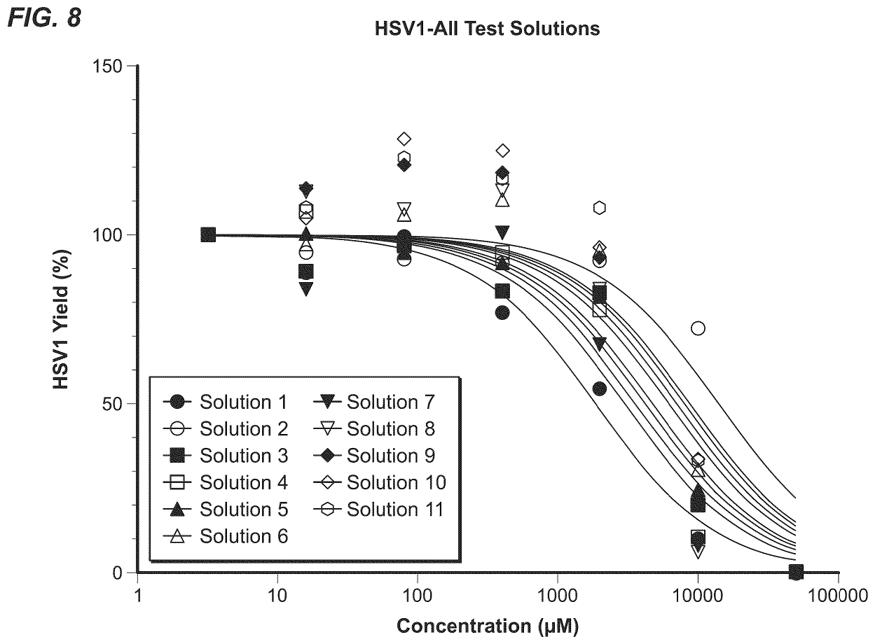

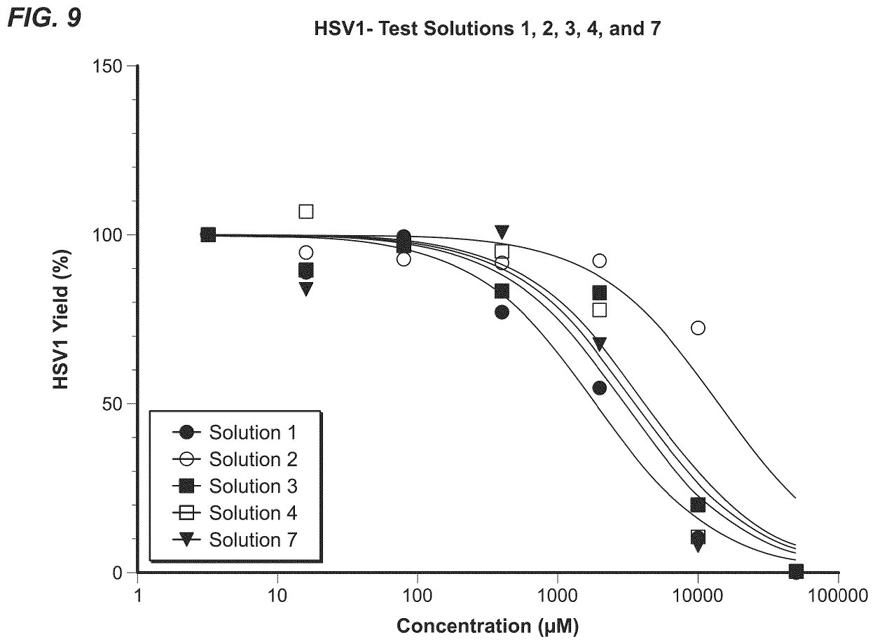

For a graphic comparison, the efficacy curves against HSV1 for test solutions 1-11 were overlayed as shown in FIG. 8. Solution 1 and Solution 7 were the most efficacious test solutions against HSV1. This can be seen more clearly in FIG. 9 showing an overlay of test Solutions 1, 2, 3, 4 and 7.

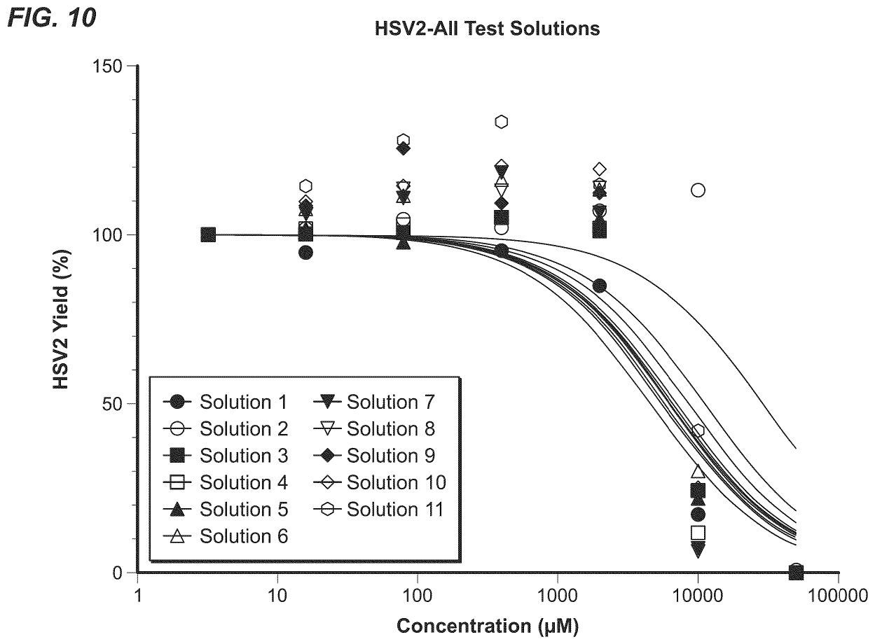

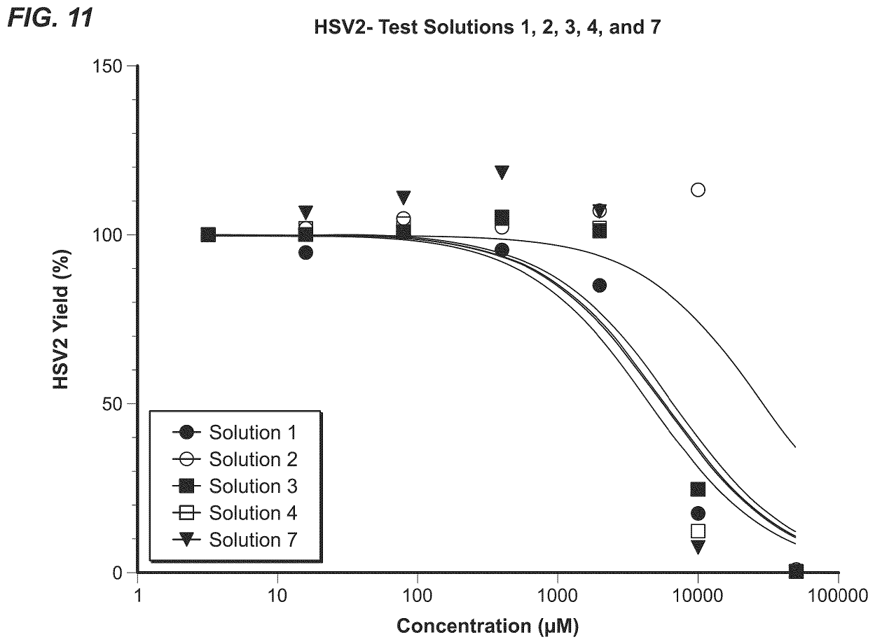

For a graphic comparison, the efficacy curves against HSV2 for test solutions 1-11 were overlayed as shown in FIG. 10. Solution 1, Solution 4, and Solution 7 were the most efficacious test solutions against HSV2. This can be seen more clearly in FIG. 11 showing an overlay of test Solutions 1, 2, 3, 4 and 7.

### DISCUSSION

In this study it was found that the original Test Composition and its different component mixtures were effective against HSV1 and HSV2, although to differing extents. Additionally, the positive control drug Acyclovir worked against both viruses, albeit the EC50 was higher than expected for HSV2. Table 10 and FIGS. 9-11 highlight the two most effective solutions tested (Solution 1 and Solution 7), as well as the different components found in each solution. The least effective component was Solution 2, which only contained L-Lysine monohydrochloride and DMSO. It is important to note that L-Lysine monohydrochloride alone was still somewhat effective with an overall SI value of 5 for HSV1 and 2.5 for HSV2. This suggests that L-Lysine monohydrochloride is at least one of the components of the original Test Composition that is active against these viruses.

### Example 6. Safety Studies of Oral Composition in Human Subjects

### Methodology:

A randomized, double-blind, placebo-controlled, 2-arm parallel group study was performed to evaluate safety of an oral test composition. This study consisted of a single 12-week/84 day study period.

The test composition product included active ingredients per capsule: L-Lysine hydrochloride 420.57 mg, Ascorbic Acid 40 mg, Calcium Ascorbate Dihydrate 91.43 mg, Niacinamide Ascorbate 42.86 mg, Ascorbyl Palmitate 7.14 mg, Hesperidin 42.86 mg, Rutin 42.86 mg, Pyridoxine Hydrochloride 7.14 mg, Threonine 7.14 mg, and Taurine 28.57 mg. Other ingredients included gelatin capsule, magnesium stearate, and silicone dioxide.

The placebo product included Ingredients: Gelatin capsule, microcrystalline cellulose, magnesium stearate.

### Administration

Participants took three oral capsules of Test Composition every morning, 30 minutes before or after eating, for 12 weeks. If a participant became symptomatic with flu-like or cold-like symptoms, they were advised to immediately start a dose of seven capsule over 8 hours for up to 48 hours (maximum 21 capsules in one 24-hour period). After the 48 hours of participant resumed the dose of three capsules.

Safety Population. Consists of all participants who received either product and on whom any post-randomization safety information was available. Number of participants included in the safety analyses: N=112.

Safety Outcomes included a comparison between Test Composition and Placebo groups in difference in vital signs, hematology, and clinical chemistry parameters and difference in the incidence of adverse events.

One hundred and twelve healthy adult human participants 18-60 yrs. of age were enrolled in the study after eligibility was assessed at screening (Visit 1) and baseline (Visit 2). Males (34%) and Females (66%). BMI 19.1-30.9 kg/m². 79% were non-smokers.

At baseline, participants were randomized into either Test Composition group or Placebo group by a blinded investigator. Participants met with study investigational team on Day 0 (Baseline, Visit 2), Day 28 (Visit 3), Day 56 (Visit 4), and Day 84 (end-of-study, Visit 5). At each visit participants blood pressure, heart rate and weight were measured, and BMI calculated. Participants completed a questionnaire at each visit, and all visits post-baseline participants' daily study diaries which included adverse events. For safety analysis Blood was collected at screening and baseline for analysis of hematology (white blood cell (WBC) count with differential, red blood cell (RBC) count, hemoglobin, hematocrit, platelet count, and RBC indices), liver function (AST, ALT, GGT and bilirubin) and kidney function (creatinine and electrolytes).

### Laboratory Methodologies

Safety endpoints of Complete Blood Count (hemoglobin, hematocrit, platelet count, red blood cell count (RBC), red cell indices, red cell distribution width (RDW), white blood cell count (WBC) and differentials (neutrophils, lymphocytes, monocytes, eosinophils, basophils), liver function (alanine aminotransferase (ALT), aspartate aminotransferase (AST), bilirubin), and kidney function laboratory blood tests (creatinine, electrolytes (Na, K, Cl)) were analysed from the blood drawn at Visit 1 (screening), and at Visit 5 by LifeLabs (London, ON, Canada) using standardized procedures.

### Statistical Tests:

Numerical efficacy endpoints were formally tested for significance between groups by Analysis of Covariance (ANCOVA). The dependent variable was the value at each visit, the factor was the study group, and the value at baseline (day 0) was the covariate.

Significant efficacy of the product, relative to placebo, was inferred if the coefficient of the study group in the ANCOVA model was significantly different from zero (p<0.05). Numerical endpoints that are intractably non-normal were assessed by the Mann-Whitney U test. A within group analysis on efficacy endpoints were done using the Student's paired t-test or, in the case of intractable non-normality, the Wilcoxon sign rank test.

Probabilities≤0.05 were considered statistically significant. All statistical analyses were completed using the R Statistical Software Package Version 3.3.3 (R Core Team, 2015) for Microsoft Windows.

Baseline demographics of participants in the Test Composition and placebo groups were well matched for age, gender, BMI, smoking status and race. One hundred and twelve participants with an age range of 18-60 years and a BMI of 19.2-30.9 kg/m² were enrolled in this study. Participants were predominantly Western European White (72%), 12% South American, 6% Eastern European White, 4% Middle Eastern, 3% South Asian, 1% Central American, and 1% South East Asian and 1% East Asian. There were 66% female participants, 79% were non-smokers, and 73% reported both occasional alcohol use and no use of alcohol. All participants were deemed healthy by physical examination and as per their screening laboratory parameters.

### Compliance:

Safety Population: Product compliance for the Safety population was 99.6% for participants in Test Composition group and 98.8% for participants in the placebo group, with no significant difference between groups.

### Safety Outcomes:

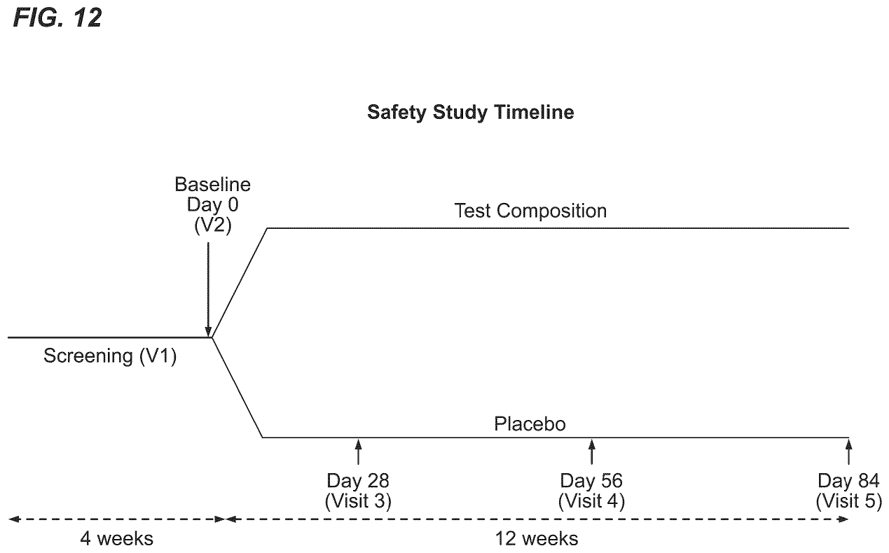

Comparisons between Test Composition and placebo in (i) difference in vital signs, hematology and clinical chemistry parameters, and (2) difference in the incidence of adverse events. A study flow timeline diagram is shown in FIG. 12.

### Safety Data for the Safety Population (N=112)

Hematology and clinical chemistry parameters were measured in-clinic at baseline and at the end of study (week 12). These parameters include complete blood count, electrolytes, creatinine, AST, ALT, GGT, and bilirubin. Data are shown in Tables 11A-D.

TABLE 11A
Hematology and Clinical Chemistry Analysis at Screening and End-of-Study of Participants in the Safety Population (N = 112).

| | | | |
|---|---|---|---|
| All Groups | Placebo | Test Composition | |
| Mean ± SD (n) | Mean ± SD (n) | Mean ± SD (n) | |
| Median (Min - Max) | Median (Min - Max) | Median (Min - Max) | Between |
| Within Group P | Within Group P | Within Group P | Group |
| Value^{δ} | Value^{δ} | Value^{δ} | P-Value |

Hemoglobin Concentration (g/L)

| | | | | | | | |
|---|---|---|---|---|---|---|---|
| Screening | 135.7 ± 11.1 | (112) | 137.9 ± 12.5 | (56) | 133.6 ± 9.1 | (56) | 0.041^{§} |
| | 134 | (117 - 164) | 136.5 | (118 - 164) | 132 | (117 - 157) | |
| Week 12 | 136.4 ± 10.9 | (112) | 138.6 ± 11.7 | (56) | 134.1 ± 9.6 | (56) | 0.031^{§} |
| End-of-Study | 134 | (113 - 162) | 139 | (116 - 160) | 133 | (113 - 162) | 0.415^{Δ} |
| Change from | 0.6 ± 6.2 | (112) | 0.7 ± 6.4 | (56) | 0.6 ± 6.0 | (56) | |
| Screening to | 0 | (−17 - 17) | 0.5 | (−17 -17) | 0 | (−12 - 16) | |

| | | | |
|---|---|---|---|
| End-of-Study | p = 0.278 | p = 0.404 | p = 0.493 |

Hematocrit (L/L)

| | | | | | | | |
|---|---|---|---|---|---|---|---|
| Screening | 0.4036 ± 0.0294 | (112) | 0.4079 ± 0.0324 | (56) | 0.3993 ± 0.0257 | (56) | 0.123^{§} |
| | 0.4 | (0.337 - 0.469) | 0.405 | (0.354 - 0.469) | 0.4 | (0.337 - 0.46) | |
| Week 12 | 0.4070 ± 0.0288 | (112) | 0.4119 ± 0.0305 | (56) | 0.4022 ± 0.0263 | (56) | 0.074^{§} |
| End-of-Study | 0.4005 | (0.35 - 0.478) | 0.41 | (0.35 - 0.463) | 0.4 | (0.35 - 0.478) | |
| Change from | 0.0035 ± 0.0182 | (112) | 0.0040 ± 0.0194 | (56) | 0.0029 ± 0.0171 | (56) | 0.361^{Δ} |
| Screening to | 0.0045 | (−0.052 - 0.046) | 0.005 | (−0.052 - 0.046) | 0.0015 | (−0.03 - 0.034) | |

| | | | |
|---|---|---|---|
| End-of-Study | p = 0.047 | p = 0.128 | p = 0.208 |

White Blood Cell Count (×E9/L)

| | | | | | | | |
|---|---|---|---|---|---|---|---|
| Screening | 6.31 ± 1.65 | (112) | 6.46 ± 1.61 | (56) | 6.16 ± 1.68 | (56) | 0.332^{§} |
| | 6 | (3.4 - 11.3) | 6.25 | (3.4 - 11.3) | 5.9 | (3.6 - 11.1) | |
| Week 12 | 5.46 ± 1.38 | (112) | 5.45 ± 1.40 | (56) | 5.47 ± 1.37 | (56) | 0.919^{§} |
| End-of-Study | 5.35 | (2.4 - 9.3) | 5.4 | (2.4 - 8.4) | 5.3 | (3 - 9.3) | |
| Change from | −0.85 ± 1.52 | (112) | −1.01 ± 1.45 | (56) | −0.68 ± 1.58 | (56) | 0.493^{Δ} |
| Screening to | −0.6 | (−6.5 - 2.8) | −0.85 | (−6.5 - 1.8) | −0.4 | (−5.4 - 2.8) | |

| | | | |
|---|---|---|---|
| End-of-Study | p < 0.001 | p < 0.001 | p = 0.002 |

Red Blood Cell Count (×E12/L)

| | | | | | | | |
|---|---|---|---|---|---|---|---|
| Screening | 4.58 ± 0.38 | (112) | 4.62 ± 0.42 | (56) | 4.54 ± 0.33 | (56) | 0.281^{§} |
| | 4.55 | (3.83 - 5.71) | 4.56 | (3.83 - 5.71) | 4.53 | (4.05 - 5.31) | |
| Week 12 | 4.61 ± 0.40 | (112) | 4.67 ± 0.43 | (56) | 4.56 ± 0.37 | (56) | 0.156^{§} |
| End-of-Study | 4.57 | (3.79 - 5.54) | 4.63 | (3.79 - 5.54) | 4.54 | (3.93 - 5.43) | |
| Change from | 0.035 ± 0.212 | (112) | 0.050 ± 0.220 | (56) | 0.020 ± 0.205 | (56) | 0.341^{Δ} |
| Screening to | 0.035 | (−0.52 - 0.75) | 0.055 | (−0.52 - 0.75) | 0.015 | (−0.4 - 0.56) | |

| | | | |
|---|---|---|---|
| End-of-Study | p = 0.082 | p = 0.092 | p = 0.468 |

Mean Corpuscular Volume (fL)

| | | | | | | | |
|---|---|---|---|---|---|---|---|
| Screening | 88.2 ± 3.8 | (112) | 88.4 ± 3.1 | (56) | 88.1 ± 4.4 | (56) | 0.673^{§} |
| | 88 | (80 - 98) | 88 | (80 - 96) | 88 | (80 - 98) | |
| Week 12 | 88.5 ± 4.0 | (112) | 88.5 ± 3.6 | (56) | 88.4 ± 4.3 | (56) | 0.906^{§} |
| End-of-Study | 88 | (78 - 99) | 89 | (78 - 98) | 88 | (79 - 99) | |
| Change from | 0.23 ± 1.38 | (112) | 0.12 ± 1.25 | (56) | 0.34 ± 1.49 | (56) | 0.425^{Δ} |
| Screening to | 0 | (−3 - 5) | 0 | (−3 - 3) | 0 | (−3 - 5) | |

| | | | |
|---|---|---|---|
| End-of-Study | p = 0.077 | p = 0.458 | p = 0.095 |

TABLE 11B
Hematology and Clinical Chemistry Analysis at Screening and End-
of-Study of Participants in the Safety Population (N = 112).

| | | | |
|---|---|---|---|
| All Groups | Placebo | Test Composition | |
| Mean ± SD (n) | Mean ± SD (n) | Mean ± SD (n) | |
| Median (Min - Max) | Median (Min - Max) | Median (Min - Max) | Between |
| Within Group P | Within Group P | Within Group P | Group |
| Value^{δ} | Value^{δ} | Value^{δ} | P-Value |

Mean Corpuscular Hemoglobin (pg)

| | | | | | | | |
|---|---|---|---|---|---|---|---|
| Screening | 29.68 ± 1.38 | (112) | 29.88 ± 1.27 | (56) | 29.47 ± 1.46 | (56) | 0.115^{§} |
| | 29.75 | (26.2 - 32.5) | 30.05 | (26.2 - 32.5) | 29.4 | (26.7 - 32.5) | |
| Week 12 | 29.59 ± 1.43 | (112) | 29.73 ± 1.39 | (56) | 29.45 ± 1.46 | (56) | 0.303^{§} |
| End-of-Study | 29.65 | (25.7 - 32.9) | 29.9 | (25.7 - 32.5) | 29.3 | (26.7 - 32.9) | |
| Change from | −0.08 ± 0.52 | (112) | −0.15 ± 0.47 | (56) | −0.02 ± 0.56 | (56) | 0.228^{Δ} |
| Screening to | −0.1 | (−1.6 - 1.7) | −0.25 | (−1 - 1.5) | 0 | (−1.6 - 1.7) | |

| | | | |
|---|---|---|---|
| End-of-Study | p = 0.091 | p = 0.020 | p = 0.814 |

Mean Corpuscular Hemoglobin Concentration (g/L)

| | | | | | | | |
|---|---|---|---|---|---|---|---|
| Screening | 336.4 ± 7.1 | (112) | 338.1 ± 7.1 | (56) | 334.8 ± 6.7 | (56) | 0.014^{§} |
| | 336 | (320 - 354) | 338 | (320 - 354) | 333.5 | (321 - 350) | |
| Week 12 | 334.9 ± 6.8 | (112) | 336.1 ± 7.6 | (56) | 333.7 ± 5.7 | (56) | 0.058^{§} |
| End-of-Study | 334 | (316 - 353) | 335 | (316 - 353) | 333 | (322 - 347) | |
| Change from | −1.5 ± 6.1 | (112) | −1.9 ± 5.8 | (56) | −1.1 ± 6.4 | (56) | 0.612^{Δ} |
| Screening to | −2 | (−28 - 14) | −2 | (−13 - 14) | −2 | (−28 - 10) | |

| | | | |
|---|---|---|---|
| End-of-Study | p = 0.011 | p = 0.017 | p = 0.209 |

Red Cell Distribution Width (%)

| | | | | | | | |
|---|---|---|---|---|---|---|---|
| Screening | 13.52 ± 0.73 | (112) | 13.46 ± 0.69 | (56) | 13.59 ± 0.78 | (56) | 0.364^{§} |
| | 13.5 | (12.4 - 16.6) | 13.5 | (12.5 - 16.6) | 13.6 | (12.4 - 16.3) | |
| Week 12 | 13.61 ± 0.73 | (112) | 13.54 ± 0.69 | (56) | 13.68 ± 0.77 | (56) | 0.327^{§} |
| End-of-Study | 13.5 | (12.4 - 16) | 13.4 | (12.5 - 16) | 13.6 | (12.4 - 15.6) | |
| Change from | 0.09 ± 0.47 | (112) | 0.08 ± 0.41 | (56) | 0.09 ± 0.52 | (56) | 0.672^{Δ} |
| Screening to | 0.1 | (−1.8 - 1.2) | 0.15 | (−1 - 0.8) | 0.1 | (−1.8 - 1.2) | |

| | | | |
|---|---|---|---|
| End-of-Study | p = 0.047 | p = 0.135 | p = 0.184 |

Platelet Count (×E9/L)

| | | | | | | | |
|---|---|---|---|---|---|---|---|
| Screening | 262 ± 58 | (111) | 261 ± 52 | (56) | 263 ± 64 | (55) | 0.845^{§} |
| | 259 | (144 - 511) | 256 | (144 - 378) | 264 | (151 - 511) | |
| Week 12 | 254 ± 55 | (112) | 257 ± 57 | (56) | 251 ± 54 | (56) | 0.559^{§} |
| End-of-Study | 249 | (109 - 421) | 250 | (139 - 421) | 249 | (109 - 408) | |
| Change from | −6.8 ± 27.8 | (111) | −3.9 ± 27.3 | (56) | −9.7 ± 28.2 | (55) | 0.275^{Δ} |
| Screening to | −6 | (−103 - 60) | −4 | (−82 - 60) | −8 | (−103 - 36) | |

| | | | |
|---|---|---|---|
| End-of-Study | p = 0.012 | p = 0.289 | p = 0.014 |

Neutrophil Count (×E9/L)

| | | | | | | | |
|---|---|---|---|---|---|---|---|
| Screening | 3.66 ± 1.29 | (112) | 3.70 ± 1.23 | (56) | 3.62 ± 1.35 | (56) | 0.613*^{§} |
| | 3.4 | (1.6 - 7.9) | 3.5 | (1.6 - 7.9) | 3.2 | (1.8 - 7.9) | |
| Week 12 | 3.05 ± 1.01 | (112) | 2.99 ± 0.96 | (56) | 3.11 ± 1.06 | (56) | 0.520*^{§} |
| End-of-Study | 3 | (0.6 - 6.8) | 3 | (0.6 - 5.5) | 3 | (1.2 - 6.8) | |
| Change from | −0.61 ± 1.25 | (112) | −0.70 ± 1.14 | (56) | −0.51 ± 1.35 | (56) | 0.348*^{Δ} |
| Screening to | −0.4 | (−4.7 - 3.3) | −0.65 | (−4.7 - 1.5) | −0.25 | (−4.7 - 3.3) | |

| | | | |
|---|---|---|---|
| End-of-Study | p < 0.001* | p < 0.001* | p = 0.004* |

Lymphocyte Count (×E9/L)

| | | | | | | | |
|---|---|---|---|---|---|---|---|
| Screening | 1.93 ± 0.60 | (112) | 2.01 ± 0.55 | (56) | 1.85 ± 0.64 | (56) | 0.080*^{§} |
| | 1.85 | (1.1 - 3.9) | 1.9 | (1.1 - 3.6) | 1.7 | (1.1 - 3.9) | |
| Week 12 | 1.76 ± 0.58 | (112) | 1.77 ± 0.52 | (56) | 1.76 ± 0.64 | (56) | 0.724*^{§} |
| End-of-Study | 1.7 | (0.9 - 4.2) | 1.7 | (0.9 - 3.3) | 1.6 | (0.9 - 4.2) | |
| Change from | −0.17 ± 0.41 | (112) | −0.24 ± 0.42 | (56) | −0.09 ± 0.38 | (56) | 0.162*^{Δ} |
| Screening to | −0.1 | (−1.7 - 1) | −0.2 | (−1.7 - 0.5) | −0.1 | (−0.9 - 1) | |

| | | | |
|---|---|---|---|
| End-of-Study | p < 0.001* | p < 0.001 | p = 0.048* |

TABLE 11C
Hematology and Clinical Chemistry Analysis at Screening and End-
of-Study of Participants in the Safety Population (N = 112).

| | | | |
|---|---|---|---|
| All Groups | Placebo | Test Composition | |
| Mean ±SD (n) | Mean ±SD (n) | Mean ± SD (n) | |
| Median (Min - Max) | Median (Min - Max) | Median (Min - Max) | Between |
| Within Group P | Within Group P | Within Group P | Group |
| Value^{δ} | Value^{δ} | Value^{δ} | P-Value |

Monocyte Count (×E9/L)

| | | | | | | | |
|---|---|---|---|---|---|---|---|
| Screening | 0.524 ± 0.160 | (112) | 0.537 ± 0.174 | (56) | 0.511 ± 0.145 | (56) | 0.378^{§} |
| | 0.5 | (0.2 - 1) | 0.5 | (0.2 - 1) | 0.5 | (0.2 - 1) | |
| Week 12 | 0.453 ± 0.132 | (112) | 0.470 ± 0.144 | (56) | 0.436 ± 0.118 | (56) | 0.175^{§} |
| End-of-Study | 0.4 | (0.2 - 0.9) | 0.4 | (0.2 - 0.9) | 0.4 | (0.2 - 0.7) | |
| Change from | −0.071 ± 0.145 | (112) | −0.068 ± 0.153 | (56) | −0.075 ± 0.139 | (56) | 0.296^{Δ} |
| Screening to | −0.1 | (−0.5 - 0.4) | −0.1 | (−0.5 - 0.3) | −0.1 | (−0.4 - 0.4) | |

| | | | |
|---|---|---|---|
| End-of-Study | p < 0.001 | p = 0.002 | p < 0.001 |

Eosinophil Count (×E9/L)

| | | | | | | | |
|---|---|---|---|---|---|---|---|
| Screening | 0.173 ± 0.152 | (112) | 0.193 ± 0.183 | (56) | 0.154 ± 0.113 | (56) | 0.347^{†} |
| | 0.1 | (0 - 0.9) | 0.1 | (0 - 0.9) | 0.1 | (0 - 0.6) | |
| Week 12 | 0.180 ± 0.152 | (112) | 0.196 ± 0.180 | (56) | 0.164 ± 0.118 | (56) | 0.687^{†} |
| End-of-Study | 0.1 | (0 - 0.9) | 0.1 | (0 - 0.9) | 0.1 | (0 - 0.6) | |
| Change from | 0.007 ± 0.094 | (112) | 0.004 ± 0.108 | (56) | 0.011 ± 0.078 | (56) | 0.552^{†} |
| Screening to | 0 | (−0.3 - 0.4) | 0 | (−0.3 - 0.4) | 0 | (−0.2 - 0.2) | |

| | | | |
|---|---|---|---|
| End-of-Study | p = 0.553^{‡} | p = 0.958^{‡} | p = 0.412^{‡} |

Basophil Count (×E9/L)

| | | | | | | | |
|---|---|---|---|---|---|---|---|
| Screening | 0.013 ± 0.034 | (112) | 0.021 ± 0.041 | (56) | 0.005 ± 0.023 | (56) | 0.013^{†} |
| | 0 | (0 - 0.1) | 0 | (0 - 0.1) | 0 | (0 - 0.1) | |
| Week 12 | 0.015 ± 0.036 | (112) | 0.023 ± 0.043 | (56) | 0.007 ± 0.026 | (56) | 0.019^{†} |
| End-of-Study | 0 | (0 - 0.1) | 0 | (0 - 0.1) | 0 | (0 - 0.1) | |
| Change from | 0.002 ± 0.038 | (112) | 0.002 ± 0.045 | (56) | 0.002 ± 0.030 | (56) | 0.992^{†} |
| Screening to | 0 | (−0.1 - 0.1) | 0 | (−0.1 -0.1) | 0 | (−0.1 - 0.1) | |

| | | | |
|---|---|---|---|
| End-of-Study | p = 0.638^{‡} | p = 0.802^{‡} | p = 0.766^{‡} |

Creatinine Concentration (μmol/L)

| | | | | | | | |
|---|---|---|---|---|---|---|---|
| Screening | 77.7 ± 14.3 | (112) | 80.6 ± 12.9 | (56) | 74.8 ± 15.1 | (56) | 0.032^{§} |
| | 76.5 | (47 - 122) | 82 | (53 - 122) | 72 | (47 - 107) | |
| Week 12 | 78.4 ± 14.4 | (112) | 81.8 ± 11.8 | (56) | 75.0 ± 16.0 | (56) | 0.012^{§} |
| End-of-Study | 77.5 | (51 - 129) | 81.5 | (52 - 105) | 75 | (51 - 129) | |
| Change from | 0.7 ± 8.7 | (112) | 1.2 ± 8.1 | (56) | 0.2 ± 9.3 | (56) | 0.187^{Δ} |
| Screening to | 2.5 | (−42 - 22) | 3 | (−30 - 14) | 2 | (−42 - 22) | |

| | | | |
|---|---|---|---|
| End-of-Study | p = 0.411 | p = 0.273 | p = 0.898 |

Sodium Concentration (mmol/L)

| | | | | | | | |
|---|---|---|---|---|---|---|---|
| Screening | 141.21 ± 1.93 | (112) | 141.09 ± 2.07 | (56) | 141.32 ± 1.79 | (56) | 0.526^{§} |
| | 141 | (137 - 146) | 141 | (137 - 146) | 141.5 | (137 - 144) | |
| Week 12 | 141.79 ± 2.31 | (112) | 141.89 ± 2.39 | (56) | 141.70 ± 2.26 | (56) | 0.655^{§} |
| End-of-Study | 142 | (135 - 146) | 142 | (135 - 146) | 141 | (137 - 146) | |
| Change from | 0.59 ± 2.67 | (112) | 0.80 ± 2.67 | (56) | 0.38 ± 2.68 | (56) | 0.551^{Δ} |
| Screening to | 1 | (−6 - 8) | 1 | (−6 - 6) | 0 | (−5 - 8) | |

| | | | |
|---|---|---|---|
| End-of-Study | p = 0.021 | p = 0.028 | p = 0.300 |

Potassium Concentration (mmol/L)

| | | | | | | | |
|---|---|---|---|---|---|---|---|
| Screening | 4.54 ± 0.41 | (112) | 4.56 ± 0.43 | (56) | 4.53 ± 0.39 | (56) | 0.698^{§} |
| | 4.5 | (3.6 - 5.4) | 4.6 | (3.6 - 5.4) | 4.5 | (3.8 - 5.4) | |
| Week 12 | 4.50 ± 0.38 | (112) | 4.53 ± 0.37 | (56) | 4.46 ± 0.39 | (56) | 0.359^{§} |
| End-of-Study | 4.4 | (3.6 - 5.5) | 4.55 | (3.7 - 5.5) | 4.4 | (3.6 - 5.5) | |
| Change from | −0.04 ± 0.53 | (112) | −0.03 ± 0.57 | (56) | −0.06 ± 0.49 | (56) | 0.380^{Δ} |
| Screening to | −0.1 | (−1.5-1.2) | −0.2 | (−1.5 - 1.2) | −0.1 | (−1.5-1.2) | |

| | | | |
|---|---|---|---|
| End-of-Study | p = 0.374 | p = 0.726 | p = 0.345 |

TABLE 11D
Hematology and Clinical Chemistry Analysis at Screening and End-
of-Study of Participants in the Safety Population (N = 112).

| | | | |
|---|---|---|---|
| All Groups | Placebo | Test Composition | |
| Mean ± SD (n) | Mean ± SD (n) | Mean ± SD (n) | |
| Median (Min - Max) | Median (Min - Max) | Median (Min - Max) | Between |
| Within Group P | Within Group P | Within Group P | Group |
| Value^{δ} | Value^{δ} | Value^{δ} | P-Value |

Chloride Concentration (mmol/L)

| | | | | | | | |
|---|---|---|---|---|---|---|---|
| Screening | 102.14 ± 2.03 | (112) | 101.88 ± 2.16 | (56) | 102.41 ± 1.88 | (56) | 0.164^{§} |
| | 102 | (97 - 106) | 102 | (97 - 106) | 103 | (97 - 106) | |
| Week 12 | 102.71 ± 2.34 | (112) | 102.48 ± 2.24 | (56) | 102.93 ± 2.43 | (56) | 0.314^{§} |
| End-of-Study | 103 | (97 - 109) | 103 | (97 - 108) | 103 | (98 - 109) | |
| Change from | 0.56 ± 2.22 | (112) | 0.61 ± 2.15 | (56) | 0.52 ± 2.30 | (56) | 0.707^{Δ} |
| Screening to | 0 | (−5 - 7) | 1 | (−4 - 5) | 0 | (−5 - 7) | |

| | | | |
|---|---|---|---|
| End-of-Study | p = 0.008 | p = 0.040 | p = 0.098 |

Bilirubin Concentration (μmol/L)

| | | | | | | | |
|---|---|---|---|---|---|---|---|
| Screening | 7.2 ± 4.0 | (112) | 7.9 ± 4.7 | (56) | 6.5 ± 3.1 | (56) | 0.111*^{§} |
| | 6 | (2 - 28) | 7 | (2 - 28) | 6 | (2 - 18) | |
| Week 12 | 8.2 ± 4.9 | (112) | 8.6 ± 4.9 | (56) | 7.7 ± 5.0 | (56) | 0.323*^{§} |
| End-of-Study | 7 | (2 - 33) | 7 | (2 - 27) | 6 | (3 - 33) | |
| Change from | 1.0 ± 3.6 | (112) | 0.7 ± 3.5 | (56) | 1.2 ± 3.7 | (56) | 0.609*^{Δ} |
| Screening to | 1 | (−9 - 15) | 1 | (−9 - 8) | 1 | (−5 - 15) | |

| | | | |
|---|---|---|---|
| End-of-Study | p = 0.017* | p = 0.207* | p = 0.031* |

Estimated Glomerular Filtration Rate(mL/min/1.73 m²)

| | | | | | | | |
|---|---|---|---|---|---|---|---|
| Screening | 93.1 ± 15.5 | (112) | 91.6 ± 14.8 | (56) | 94.6 ± 16.3 | (56) | 0.309^{§} |
| | 92 | (60 - 121) | 91 | (60-121) | 98 | (62- 121) | |
| Week 12 | 91.7 ± 16.7 | (112) | 88.7 ± 14.9 | (56) | 94.7 ± 17.9 | (56) | 0.054^{§} |
| End-of-Study | 92 | (45 - 121) | 90.5 | (58 - 121) | 98 | (45 - 121) | |
| Change from | −1.5 ± 10.4 | (112) | −3.0 ± 9.0 | (56) | 0.1 ± 11.5 | (56) | 0.068^{Δ} |
| Screening to | −2 | (−30 - 53) | −5 | (−20 - 26) | −1.5 | (−30 - 53) | |

| | | | |
|---|---|---|---|
| End-of-Study | p = 0.142 | p = 0.016 | p = 0.963 |

Aspartate Transaminase (U/L)

| | | | | | | | |
|---|---|---|---|---|---|---|---|
| Screening | 17.8 ± 4.0 | (111) | 18.6 ± 4.1 | (55) | 17.0 ± 3.9 | (56) | 0.026*^{§} |
| | 17 | (10 - 30) | 18 | (11 - 30) | 17 | (10 - 30) | |
| Week 12 | 19.4 ± 5.8 | (112) | 18.8 ± 6.2 | (56) | 20.1 ± 5.4 | (56) | 0.150*^{§} |
| End-of-Study | 18 | (8 - 46) | 18 | (8 - 46) | 18.5 | (12 - 36) | |
| Change from | 1.6 ± 4.8 | (111) | 0.2 ± 5.0 | (55) | 3.0 ± 4.1 | (56) | <0.001*^{Δ} |
| Screening to | 2 | (−10 - 20) | 1 | (−10 - 20) | 2 | (−5 - 15) | |

| | | | |
|---|---|---|---|
| End-of-Study | p = 0.002* | p = 0.729* | p < 0.001* |

Alanine Transaminase (U/L)

| | | | | | | | |
|---|---|---|---|---|---|---|---|
| Screening | 19.5 ± 9.6 | (112) | 21.0 ± 11.6 | (56) | 17.9 ± 7.0 | (56) | 0.214*^{§} |
| | 18 | (7 - 64) | 18.5 | (7 - 64) | 17 | (8 - 41) | |
| Week 12 | 22.3 ± 14.6 | (112) | 22.1 ± 17.6 | (56) | 22.6 ± 10.9 | (56) | 0.326*^{§} |
| End-of-Study | 19 | (7 - 112) | 17 | (7 - 112) | 19.5 | (8 - 55) | |
| Change from | 2.9 ± 10.2 | (112) | 1.1 ± 11.8 | (56) | 4.6 ± 8.1 | (56) | 0.004*^{Δ} |
| Screening to | 1 | (−37 - 55) | 0 | (−37 - 55) | 2 | (−7 - 35) | |

| | | | |
|---|---|---|---|
| End-of-Study | p = 0.003 | p = 0.957* | p < 0.001 |

Gamma-glutamyl transferase (U/L)

| | | | | | | | |
|---|---|---|---|---|---|---|---|
| Screening | 19.5 ± 14.4 | (112) | 18.7 ± 12.9 | (56) | 20.3 ± 15.9 | (56) | 0.618*^{§} |
| | 15 | (6 - 104) | 14 | (7 - 65) | 15.5 | (6 - 104) | |
| Week 12 | 19.8 ± 17.0 | (112) | 18.4 ± 13.9 | (56) | 21.3 ± 19.6 | (56) | 0.312*^{§} |
| End-of-Study | 14.5 | (5 - 130) | 14 | (5 - 79) | 16.5 | (6 - 130) | |
| Change from | 0.4 ± 11.5 | (112) | −0.3 ± 5.5 | (56) | 1.0 ± 15.4 | (56) | 0.243*^{Δ} |
| Screening to | 0 | (−39 - 98) | 0 | (−21 - 15) | 1 | (−39 - 98) | |

| | | | |
|---|---|---|---|
| End-of-Study | p = 0.769* | p = 0.243* | p = 0.625* |

fL, femtoliter; g, G gram; L, liter; Max, maximum; m, meters; μg, microgram; μmol, micromoles; mL, milliliter; mmol, millimoles; Min, minimum; min, minutes; nmol, nanomoles; N, number; % Percent; pg, picogram; SD, standard deviation; U, units.
^{§}Between group comparisons were made using the independent Student t-test.
^{†}Between group comparisons were made using the Mann-Whitney U test.
^{Δ}Between group comparisons were made using ANCOVA adjusting for screening.
^{δ}Within group comparisons were made using the paired Student t-test.
^{‡}Within group comparisons were made using the non-parametric Wilcoxon signed-rank test.
*Logarithmic transformation was required to achieve normality.
Probability values P ≤ 0.05 are statistically significant.

### Safety Conclusions

### Hematology and Clinical Chemistry

There were no significant between-group differences in the change in levels of the hematological panel and WBC differential. However, clinical chemistry analysis for liver parameters, aspartate transaminase (p<0.001) and alanine transaminase (p=0.004), showed significant differences between the Test Composition and Placebo groups at the end of the study. These changes in liver parameters were not deemed of clinical significance as per the Medical Director/Qualified Investigator (QI).

Within groups, a significant decrease from baseline in mean corpuscular hemoglobin was observed at the end of the study in the placebo group (p=0.020) but not in the Test Composition group. Correspondingly, there was a significant decrease from baseline in mean corpuscular hemoglobin concentration at the end of the study in the placebo group (p=0.017) but not in the Test Composition group, as shown in Table 11B.

Within groups, a significant decrease from baseline in WBC count was observed at the end of the study in the Test Composition group (p=0.002) and the placebo group (p<0.001), as shown in Table 11A.

Within groups, the WBC differential showed a significant decrease in the Test Composition Support group for neutrophil count (p=0.004), lymphocyte count (p=0.048) and monocyte count (p<0.001). Significant within group decreases in the placebo group were observed in neutrophil count (p<0.001), lymphocyte count (p<0.001) and monocyte count (p=0.002), as shown in Tables 11B and 11C, respectively.

Within groups, the platelet counts significantly decreased (p=0.014) in the Test Composition group but not in the placebo group.

Within groups, clinical chemistry analysis showed a significant increase in the Test Composition group for bilirubin concentration (p=0.031), aspartate transaminase and alanine transaminase concentration (p<0.001 for both), as shown in Table 11D. Significant within group increases in the placebo group were observed in sodium concentration (p=0.028), chloride concentration (p=0.040) and a decrease in EGFR (p=0.016) at the end of the study, as shown in Tables 11C and 11D, respectively.

All measurements and changes in the hematology and clinical chemistry parameters were deemed as not clinically significant by the QI.

### Vital Signs and Anthropometric Measurements

Mean systolic blood pressure, mean diastolic blood pressure, heart rate, and weight for BMI calculations were measured in-clinic at baseline, week 4, week 8, and week 12 for all participants in the safety population (N=112). There were no significant between-group differences in vital signs and anthropometric measures at any timepoint during the study.

### Incidence of Adverse Events

There was a total of 44 adverse events (AEs) reported by 33 participants. Of these, 21 were reported by 16 participants in the Test Composition group and 23 AEs were reported by 17 participants in the Placebo group.

Of the 21 AEs reported by those in the Test Composition group, five were assessed as possibly related to the product. The possibly related AEs were gastrointestinal disorders (N=4) and skin and subcutaneous tissue disorders (N=1). All other AEs were assessed as unlikely or not related to the product.

Of the 23 AEs reported by those in the placebo group, two were assessed as possibly related to the product. The possibly related AEs were gastrointestinal disorders (N=2). All other AEs were assessed as either unlikely or not related to the product.

All AEs resolved by the end of the study.

### Safety Conclusions

In this 12-week study, Test Composition was generally safe and well-tolerated. There were no causal relationships established between Test Composition and the AEs. No allergic reactions to the ingredients in test Composition were reported by the participants in this study.

### Clauses

Clause 1. A method of treating, preventing, preventing recurrence, or preventing reactivation of a herpesvirus infection in a subject in need thereof comprising administering to the subject a therapeutically effective amount of a composition comprising a lysine, an ascorbic compound, a flavonoid glycoside, and a threonine.

Clause 2. The method of clause 1, wherein the herpesvirus is selected from the group consisting of herpes simplex type 1 (HSV-1), herpes simplex type 2 (HSV-2), varicella zoster virus (VZV), Epstein-Barr virus (EBV), human cytomegalovirus (HCMV), human herpesvirus 6A (HHV-6A), human herpesvirus 6B (HHV-6B), human herpesvirus 7 (HHV-7), and human herpesvirus 8 (HHV-8).

Clause 3. The method of clause 1 or 2, wherein the composition further comprises a taurine.

Clause 4. The method of any one of clauses 1-3, wherein the composition further comprises a pyridoxine.

Clause 5. The method of any one of clauses 1-4, wherein the lysine is selected from L-lysine, L-lysine monohydrochloride, L-lysine dihydrochloride, L-lysine succinate, L-lysine glutamate, and L-lysine orotate.

Clause 6. The method of any one of clauses 1-5, wherein the ascorbic compound is selected from the group consisting of ascorbic acid, dehydroascorbic acid, calcium ascorbate, magnesium ascorbate, potassium ascorbate, sodium ascorbate, manganese ascorbate, zinc ascorbate, iron ascorbate, copper ascorbate, boron ascorbate, molybdenum ascorbate, chromium ascorbate, ascorbyl palmitate, ascorbyl arachidonate, ascorbyl stearate, ascorbyl linoleate, ascorbyl linoleneate, and ascorbyl oleate.

Clause 7. The method of any one of clauses 1-6, wherein the flavonoid glycoside is selected the group consisting of hesperidin, rutin, naringin, and quercitrin, optionally wherein the wherein the flavonoid glycoside comprises hesperidin and rutin.

Clause 8. The method of any one of clauses 4-7, wherein the pyridoxine is selected from the group consisting of pyridoxine, pyridoxine hydrochloride, pyridoxine phosphate, pyridoxal, pyridoxal hydrochloride, pyridoxal 5-phosphate, pyridoxic acid, pyridoxamine, pyridoxamine hydrochloride, and pyridoxamine dihydrochloride.

Clause 9. The method of any one of clauses 1-8, wherein the composition comprises L-lysine monohydrochloride, ascorbic acid or dehydroascorbic acid, calcium ascorbate, niacinamide ascorbate, ascorbyl palmitate, hesperidin, rutin, pyridoxine hydrochloride, threonine, and taurine.

Clause 10. A method of reducing herpesvirus replication in a cell comprising exposing a virus-infected cell to an effective amount of a composition comprising a lysine, an ascorbic compound, a flavonoid glycoside, and a threonine, optionally further comprising a pyridoxine and further optionally comprising a taurine.

Clause 11. An anti-herpesvirus supplement oral dosage form comprising a composition comprising a therapeutically effective amount of a lysine, an ascorbic compound, a flavonoid glycoside, and a threonine, optionally further comprising a taurine and/or a pyridoxine.

Clause 12. The anti-herpesvirus dosage form according to clause 11, wherein the anti-herpesvirus effective amount of the lysine is in a single dose range of from about 1,000 mg to about 5,000 mg, or about 1,250 mg to about 3,500 mg, or about 2,000 mg to about 3,500 mg.

Clause 13. The anti-herpesvirus dosage form of clause 11 or 12, wherein the dosage form is in a form selected from the group consisting of powder, capsule, lozenge, troche, gummy, tablet, orally disintegrating tablet, liquid, and caplet.

Clause 14. The anti-herpesvirus dosage form of any one of clauses 11-13, wherein the ascorbic compound is selected from the group consisting of ascorbic acid, dehydroascorbic acid, calcium ascorbate, niacinamide ascorbate, and ascorbyl palmitate.

Clause 15. The anti-herpesvirus dosage form of any one of clauses 11-14, further comprising one or more additives selected from the group consisting of vehicle, binder, disintegrating agent, lubricant, thickener, surfactant, osmotic pressure regulator, electrolyte, sweetener, flavoring, perfume, pigment, and pH regulator.

Clause 16. The anti-herpesvirus dosage form of clause 15, wherein the disintegrating agent is selected from the group consisting of microcrystalline cellulose, carboxymethylcellulose (CMC), CMC-Na, CMC-Ca, and croscarmellose sodium.

Clause 17. The anti-herpesvirus dosage form of clause 15, wherein the lubricant is selected from the group consisting of leucine, isoleucine, valine, sugar-ester, hardening oil, stearic acid, magnesium stearate, talc, and macrogol.

Clause 18. The anti-herpesvirus dosage form of clause 15, wherein the sweetener is selected from the group consisting of glucose, fructose, maltose, sucrose, xylose, lactose, xylitol, sorbitol, mannitol, maltitol, xylitol, coupling sugar, paratinose, glycerin, erythritol, dextrin, cyclodextrin, fructo-oligosaccharide, galacto-oligosaccharide, lacto-sucrose, thaumatin, stevia, stevia extract, rebaudioside A, glycyrrhizinic acid, saccharin, alitame, and aspartame.

Clause 19. The anti-herpesvirus dosage form of any one of clauses 11-18, wherein a single dose comprises from about 1.2 g to about 3.5 g L-lysine monohydrochloride; from about 0.1 g to about 1.5 g ascorbic acid or dehydroascorbic acid; from about 0.2 g to about 0.8 g hesperidin; from about 0.1 g to about 0.5 g rutin; from about 0.01 g to about 0.08 g threonine; optionally from about 0.02 g to about 0.08 g pyridoxine hydrochloride; and optionally from about 0.02 g to about 0.4 g taurine.

Clause 20. The dosage form of clause 19, further comprising from about 0.5 g to about 0.75 g calcium ascorbate; from about 0.1 g to about 0.5 g niacinamide ascorbate; and from about 0.01 g to about 0.1 g ascorbyl palmitate.

Clause 21. A method of reducing frequency of recurrence of a herpesvirus infection in a subject comprising treating the subject with an effective amount of a composition comprising a therapeutically effective amount of a lysine, an ascorbic compound, a flavonoid glycoside, and a threonine, optionally further comprising a taurine, and/or a pyridoxine.

Clause 22. A method of decreasing severity and/or duration of symptoms of a herpesvirus infection in a subject comprising treating the subject with an effective amount of a composition comprising a therapeutically effective amount of a lysine, an ascorbic compound, a flavonoid glycoside, and a threonine, optionally further comprising a taurine, and/or a pyridoxine.

Clause 23. A composition for the manufacture of a medicament for treating a herpesvirus infection, the composition comprising a lysine, an ascorbic compound, a flavonoid glycoside, and a threonine, optionally further comprising a pyridoxine, and optionally further comprising a taurine.

Clause 24. A composition for treating or preventing a herpesvirus infection comprising a therapeutically effective amount of a lysine, an ascorbic compound, a flavonoid glycoside, and a threonine, optionally further comprising a taurine, and optionally further comprising a pyridoxine.

Clause 25. A method of reducing risk of or slowing developing Alzheimer's disease or dementia associated with a herpesvirus infection or reactivation of a herpesvirus infection in a subject, the method comprising treating the subject with an effective amount of a composition comprising a therapeutically effective amount of a lysine, an ascorbic compound, a flavonoid glycoside, a threonine, optionally a taurine, and optionally a pyridoxine.

Clause 26. A composition for reducing risk of or slowing development of Alzheimer's disease or dementia associated with a herpesvirus infection or reactivation in a subject, the composition comprising an effective amount of a composition comprising a therapeutically effective amount of a lysine, an ascorbic compound, a flavonoid glycoside, a threonine, optionally a taurine, and optionally a pyridoxine.

Clause 27. The method of clause 25 or composition of claim 26, wherein the lysine is selected from L-lysine, L-lysine monohydrochloride, L-lysine dihydrochloride, L-lysine succinate, L-lysine glutamate, and L-lysine orotate.

Clause 28. The method or composition of any one of claims 25-27, wherein the ascorbic compound is selected from the group consisting of ascorbic acid, dehydroascorbic acid, calcium ascorbate, magnesium ascorbate, potassium ascorbate, sodium ascorbate, manganese ascorbate, zinc ascorbate, iron ascorbate, copper ascorbate, boron ascorbate, molybdenum ascorbate, chromium ascorbate, ascorbyl palmitate, ascorbyl arachidonate, ascorbyl stearate, ascorbyl linoleate, ascorbyl linoleneate, and ascorbyl oleate.

Clause 29. The method or composition of any one of claims 25-28, wherein the flavonoid glycoside is selected the group consisting of hesperidin, rutin, naringin, and quercitrin, optionally wherein the wherein the flavonoid glycoside comprises hesperidin and rutin.

Clause 30. The method or composition of any one of claims 25-29, wherein the pyridoxine is selected from the group consisting of pyridoxine, pyridoxine hydrochloride, pyridoxine phosphate, pyridoxal, pyridoxal hydrochloride, pyridoxal 5-phosphate, pyridoxic acid, pyridoxamine, pyridoxamine hydrochloride, and pyridoxamine dihydrochloride.

Clause 31. The method or composition of any one of clauses 25-30, wherein the threonine is L-threonine or a pharmaceutically acceptable salt thereof.

Clause 32. The method or composition of any one of clauses 25-31, wherein the anti-herpesvirus effective amount of the lysine is in a single dose range of from about 1,000 mg to about 5,000 mg, or about 1,250 mg to about 3,500 mg, or about 2,000 mg to about 3,500 mg.

Clause 33. The method or composition of any one of clauses 25-32, wherein a single dose of the composition comprises from about 1.2 g to about 3.5 g L-lysine monohydrochloride; from about 0.1 g to about 1.5 g ascorbic acid or dehydroascorbic acid; from about 0.2 g to about 0.8 g hesperidin; from about 0.1 g to about 0.5 g rutin; from about 0.01 g to about 0.08 g threonine; optionally from about 0.02 g to about 0.08 g pyridoxine hydrochloride; and optionally from about 0.02 g to about 0.4 g taurine.

Clause 34. The method or composition of clause 33, further comprising from about 0.5 g to about 0.75 g calcium ascorbate; from about 0.1 g to about 0.5 g niacinamide ascorbate; and from about 0.01 g to about 0.1 g ascorbyl palmitate.

The above specification, examples and data provide a complete description of the manufacture and use of the composition of the invention. Since many embodiments of the invention can be made without departing from the spirit and scope of the invention, the invention resides in the claims hereinafter appended.

What is claimed is:

1. A method of treating, preventing, preventing recurrence, or preventing reactivation of a herpesvirus infection in a subject in need thereof, the method comprising administering to the subject a therapeutically effective amount of a composition comprising a lysine, an ascorbic compound, a flavonoid glycoside, and a threonine.

2. The method of claim 1, wherein the composition further comprises a taurine.

3. The method of claim 1, wherein the composition further comprises a pyridoxine, optionally wherein the pyridoxine is selected from the group consisting of pyridoxine, pyridoxine hydrochloride, pyridoxine phosphate, pyridoxal, pyridoxal hydrochloride, pyridoxal 5-phosphate, pyridoxic acid, pyridoxamine, pyridoxamine hydrochloride, and pyridoxamine dihydrochloride.

4. The method of claim 1, wherein the herpesvirus is selected from the group consisting of herpes simplex type 1 (HSV-1), herpes simplex type 2 (HSV-2), varicella zoster virus (VZV), Epstein-Barr virus (EBV), human cytomegalovirus (HCMV), human herpesvirus 6A (HHV-6A), human herpesvirus 6B (HHV-6B), human herpesvirus 7 (HHV-7), and human herpesvirus 8 (HHV-8), optionally wherein the herpesvirus is a herpes simplex virus.

5. The method of claim 1, wherein the lysine is selected from L-lysine, L-lysine monohydrochloride, L-lysine dihydrochloride, L-lysine succinate, L-lysine glutamate, and L-lysine orotate.

6. The method of claim 1, wherein the ascorbic compound is selected from the group consisting of ascorbic acid, dehydroascorbic acid, calcium ascorbate, magnesium ascorbate, potassium ascorbate, sodium ascorbate, manganese ascorbate, zinc ascorbate, iron ascorbate, copper ascorbate, boron ascorbate, molybdenum ascorbate, chromium ascorbate, ascorbyl palmitate, ascorbyl arachidonate, ascorbyl stearate, ascorbyl linoleate, ascorbyl linolenate, and ascorbyl oleate.

7. The method of claim 1, wherein the flavonoid glycoside is selected the group consisting of hesperidin, rutin, naringin, quercitrin, and a pharmaceutically acceptable salt thereof.

8. The method of claim 1, wherein the threonine is L-threonine or a pharmaceutically acceptable salt thereof.

9. The method of claim 1, wherein the composition comprises L-lysine monohydrochloride, ascorbic acid, calcium ascorbate, niacinamide ascorbate, ascorbyl palmitate, hesperidin, rutin, pyridoxine hydrochloride, threonine, and taurine.

10. A method of reducing herpesvirus replication in a cell comprising exposing a virus-infected cell to an effective amount of a composition comprising a lysine, an ascorbic compound, a flavonoid glycoside, a threonine, optionally a pyridoxine, and optionally a taurine.

11. An anti-herpesvirus supplement composition or dosage form comprising therapeutically effective amounts of a lysine, an ascorbic compound, a flavonoid glycoside, a threonine, optionally a taurine, and optionally a pyridoxine.

12. The anti-herpesvirus supplement dosage form of claim 11, wherein the dosage form is an oral dosage form or a topical dosage form.

13. The anti-herpesvirus oral dosage form according to claim 12, wherein the anti-herpesvirus effective amount of the lysine is in a single dose range of from about 1,000 mg to about 5,000 mg.

14. The anti-herpesvirus oral dosage form of claim 13, comprising from about 1.2 g to about 3.5 g L-lysine monohydrochloride, from about 0.1 g to about 1.5 g ascorbic acid or dehydroascorbic acid; from about 0.2 g to about 0.8 g hesperidin; from about 0.1 g to about 0.5 g rutin; from about 0.01 g to about 0.08 g threonine; optionally from about 0.02 g to about 0.08 g pyridoxine hydrochloride or a pyridoxal; and optionally from about 0.02 g to about 0.4 g taurine.

15. The anti-herpesvirus oral dosage form of claim 14, further comprising from about 0.5 g to about 0.75 g calcium ascorbate; from about 0.1 g to about 0.5 g niacinamide ascorbate; and from about 0.01 g to about 0.1 g ascorbyl palmitate.

16. The anti-herpesvirus oral dosage form of claim 12, wherein the oral dosage form is in a form selected from the group consisting of powder, capsule, lozenge, troche, gummy, tablet, orally disintegrating tablet, liquid, and caplet.

17. The anti-herpesvirus oral dosage form of claim 12, further comprising one or more additives selected from the group consisting of vehicle, binder, disintegrating agent, lubricant, thickener, surfactant, osmotic pressure regulator, electrolyte, sweetener, flavoring, perfume, pigment, and pH regulator.

18. A method of reducing frequency, recurrence, or reactivation of a herpesvirus infection in a subject, the method comprising treating the subject with an effective amount of the dosage form or composition according to claim 11.

19. A method of decreasing severity and/or duration of symptoms of a herpesvirus infection in a subject comprising treating the subject with an effective amount of the dosage form or composition according to claim 11.

20. A method of reducing risk of or slowing development of Alzheimer's disease or dementia associated with a herpesvirus infection or reactivation in a subject, the method comprising treating the subject with an effective amount of the dosage form or composition according to claim 11.
